SEQUENCE LISTING (94 sequences)

SEQ ID NO: 1 - DNA, 826 bp, Oryza sativa
tggtaccgccacgccaaggtggtaaggatggttgtcagcacttaccaagcagcaagtggtgctggggctgcggccatggaagaactcaaacttcaaactcaagaggtcttggcggggaaagcaccaacatgcaacattttcagtcagcagtatgcttttaatatattttcacataatgcaccaattgttgaaaatgggtacaatgaggaggagatgaagatggtgaaggagaccagaaaaatctggaatgataaagatgtgaaggtaactgcaacctgcatacgagttcctgtgatgcgtgcacatgctgaaagtgtgaatctacagtttgaaaagccacttgatgaggatactgcaagggaaatcttgagggcagctgaaggtgttaccattattgatgaccgtgcttccaatcgcttccccacacctcttgaggtatcggataaagatgatgtagcagtgggtagaattcgtcaggatttgtcgcaagatgataacaaagggctggacatatttgtttgtggagatcaaatacgtaaaggtgctgcactcaatgctgtgcagattgctgaaatgctactcaagtgattttcttttctgtacctttctctccttgcccctctttgctctagtcattgtttgacggatgtactctggttagtatgagatcaattttgatcatcttttgtaatctatattcctagtgaaataaatgtaaaacggttttgctctatcttctgcacaagtgtagaagaaatctgaaattgggaaattggagtgtggcccttgttcaaaaaaaaaaaaaaaaaaaaaaaaaaaaaaaaaa

SEQ ID NO: 2 - protein, 195 aa, Oryza sativa
WYRHAKVVRMVVSTYQAASGAGAAAMEELKLQTQEVLAGKAPTCNIFSQQYAFNIFSHNAPIVENGYNEEEMKMVKETRKIWNDKDVKVTATCIRVPVMRAHAESVNLQFEKPLDEDTAREILRAAEGVTIIDDRASNRFPTPLEVSDKDDVAVGRIRQDLSQDDNKGLDIFVCGDQIRKGAALNAVQIAEMLLK

SEQ ID NO: 3 - DNA, 875 bp, Triticum aestivum
cctcatggctgtcacgccgctgcatcgccacgccaaggtgaaaaggatggttgtcagcacataccaagcagcaagtggtgctggtgctgcagccatggaagaactcaaacttcagactcgagaggtcttggaaggaaagccaccaacctgtaacattttcagtcaacagtatgcttttaatatattttcgcataatgcacctattgttgaaaatggctataatgaggaagagatgaaaatggtgaaggagaccagaaaaatctggaatgacaaggatgtaagagtaactgcaacttgtatacgggttcctacgatgcgcgcgcatgccgaaagcgtgaatctacagtttgaaaagccacttgatgaggacactgccagagaaatcttgagggcagctcctggtgttaccattagtgacgaccgtgctgccaaccgcttccctacaccactggaggtatcggataaagatgacgtatcagttggtaggattcgccaggacttgtcacaagatgataacagagggttggagttatttgtctgtggagaccagatacgtaaaggcgccgcgctgaacgctgtgcagattgctgaaatgctactgaagtgaccgcctttttaccattgtctcatgtgccacgttgctctatccattgatggattgatgtactctagtcactttcaacccagttttggtcgtcgtcttttttgtaatctgtcaacctagcagaagaagtgtaagacgggctttagtcatctgttgcacacaaaagtgcagccacaagtttagaaaaggagggttttcacttgttcggattttgccttaggttggactttgttgcaagttgtcgtttgtttcttgaaagctggtctgctgt

SEQ ID NO: 4 - protein, 201 aa, Triticum aestivum
LMAVTPLHRHAKVKRMVVSTYQAASGAGAAAMEELKLQTREVLEGKPPTCNIFSQQYAFNIFSHNAPIVENGYNEEEMKMVKETRKIWNDKDVRVTATCIRVPTMRAHAESVNLQFEKPLDEDTAREILRAAPGVTISDDRAANRFPTPLEVSDKDDVSVGRIRQDLSQDDNRGLELFVCGDQIRKGAALNAVQIAEMLLK

SEQ ID NO: 5 - DNA, 457 bp, Glycine max
  unsure (211): n = a, c, g or t
  unsure (320): n = a, c, g or t
  unsure (377): n = a, c, g or t
  unsure (391)..(392): n = a, c, g or t
  unsure (410): n = a, c, g or t
  unsure (420): n = a, c, g or t
  unsure (428): n = a, c, g or t
  unsure (432): n = a, c, g or t
  unsure (434): n = a, c, g or t
  unsure (438): n = a, c, g or t
  unsure (442): n = a, c, g or t
  unsure (451): n = a, c, g or t
gtctgttttaaaatccaacacttaatctctctcttcgcagcctaaaatcccaatggcttcactctctgttttgcgccacaaccacctcttctcgggccccctcccggcccgccccaagcccacctcctcctcctcctccaggatccgaatgtccctccgcgagaacggcccctccatcgccgtcgtgggcgtcaccggcgccgtcggccangagttcctctccgtcctctccgaccgcgacttcccctaccgctccattcatatgctggcttccaagcgctccgctggacgccgcatcaccttcgaggacagggactacntcttcaggagctcacgccggagagttcgacggtgtcgacatcgcgctcttcagcgcngggggtccatcaannaagcattcggaccatcgncgtaaatcgngggacggncgtngncaanatanctccggttncctttg

SEQ ID NO: 6 - protein, 86 aa, Glycine max
MASLSVLRHNHLFSGPLPARPKPTSSSSSRIRMSLRENGPSIAVVGVTGAVGQEFLSVLSDRDFPYRSIHMLASKRSAGRRITFED

SEQ ID NO: 7 - protein, 160 aa, Legionella pneumophila
MSRHLNVAIVGATGAVGETFLTVLEERNFPIKSLYPLASSRSVGKTVTFRDQELDVLDLAEFDFSKVDLALFSAGGAVSKEYAPKAVAAGCVVVDNTSCFRYEDDIPLVVPGSESSSNRDYTKRGIIANPNCSTIQMVVALKPIYDAVGISRINVATYQS

SEQ ID NO: 8 - DNA, 1054 bp, Zea mays
atttaacggaaatgggaagacactcgaacatcttaaattagctgctgagagtggagtatttgtaaatgtggatagcgaatttgatttggagaatattgtcagagctgcaagagctactggaaagaaagtgcctgttttgcttcgaataaatccagatgtggatccgcaggtacatccttatgttgccacgggaaataaaacgtctaaatttgggatccgcaatgagaaattgcaatggtttttggactctatcaagtcatacccgaatgaaatcaaactcgttggtgttcattgccatctgggatctactattacaaaggttgatatattcagagatgctgcagttcttatgctgaattatgtcgatgaaattcgagcacaaggttttaagttggagtacctgaatatcggaggtggtttgggaatagattaccatcataccgatgcagtcttacctacacctatggatctcatcaacactgtgcgagaattagttctctctcaagatctcactcttattattgaacccggaagatccttgattgctaatacttgctgcttcgtcaatagagtaactggtgttaaatctaatggtacaaagaatttcattgttgttgatggcagcatggcagaactcatcagacctagtctgtatggagcataccagcatatcgaactggtctctccccccactcctggtgctgaagcagcgaccttcgatattgttggaccagtttgtgagtctgcagatttccttggaaaagatagggaacttccaacacctgatgagggagctggactggttgttcatgatgcaggtgcctactgcatgagcatggcttccacctacaacctgaagttgaggccaccggaatactgggtggaagcggacggttcgatcgttaagatcaggcatggagagaagcttgatgactacatgaagttctttgatggtcttcctgcttagatgtttattatctgcgactgctacggacgatgttttcttggggataattggattttctttgtcaaaaaaaaaaaaaaaaaaaaaaaa

SEQ ID NO: 9 - protein, 321 aa, Zea mays
FNGNGKTLEHLKLAAESGVFVNVDSEFDLENIVRAARATGKKVPVLLRINPDVDPQVHPYVATGNKTSKFGIRNEKLQWFLDSIKSYPNEIKLVGVHCHLGSTITKVDIFRDAAVLMLNYVDEIRAQGFKLEYLNIGGGLGIDYHHTDAVLPTPMDLINTVRELVLSQDLTLIIEPGRSLIANTCCFVNRVTGVKSNGTKNFIVVDGSMAELIRPSLYGAYQHIELVSPPTPGAEAATFDIVGPVCESADFLGKDRELPTPDEGAGLVVHDAGAYCMSMASTYNLKLRPPEYWVEADGSIVKIRHGEKLDDYMKFFDGLPA

SEQ ID NO: 10 - DNA, 1813 bp, Zea mays
cgcttcctggaaggctggaacagaaagaaccctaaaccctagcaatggcggcggcgaacctgctgtcgcgctcccttctccccaccccaaacactatccgaacgagccaccccaccccgcggagcccagccgtcgtctccttcccccgccgccgtgcccgcctgtccgtgtgcgcctccgtctccatggcctccccgtccccaccgccacagcccgcggcggccggcgtgccgaagcactgcttccggcgcggcgccgacggctacctgtactgcgagggagtgagggtggaagacgcgatggcggctgccgagcgcagccccttctatctctacagcaagcttcagatcctccgcaacttcgccgcttaccgcgacgctctccaggggctccgctccatcgtcgggtatgccgtgaaggccaacaataacctccccgtgctacgcgtcctgcgtgagcttggctgcggcgccgtcctcgtcagcggcaacgagctccgactcgccctccaggcgggattcgaccccgccaggtgtatatttaacggaaatgggaagacactcgaagatcttaaattggctgctgagagtggagtatttgtaaatgtggatagtgaatttgatttagagaatattgtcagagctgcaagagctactggaaagaaagtgcctgttttacttagaataaatccagatgtggatccacaggtacatccatatgttgccacgggaaataaaacatccaaattcgggatccgcaatgagaaattgcaatggtttttgaactctatcaagtcatactcgaatgaaatcaaactcgttggtgttcattgccatctgggatctactattacaaaggttgatatattcagagatgctgcagtgcttatggtgaattatgtcgatgaaattcgagcacaaggttttaagttggagtacctgaatattggaggtggtttgggaatagattaccatcataccgatgcagtcttacctacacctatggatctcatcaacactgtacgagaattagttctctctcaagatcttactcttattattgaacctggaagatccttgattgctaatacttgctgcttcgtcaatagagtaactggtgttaaatctaatggtacaaagaatttcattgttgttgatggcagcatggcagaactcatcagacctagcctgtatggagcatatcagcatatcgaattggtctctccccccactcctggtgctgaagtagcgaccttcgatattgttgggccagtttgtgagtctgcagatttccttggaaaagatagggaacttccaacacctgatgagggagctggactggttgttcatgatgcaggtgcctactgcatgagcatggcttccacctacaacctgaagttgaggccgccagagtactgggttgaagaggatggttcgattgttaagatcaggcatgaagagaagctcgatgactacatgaagttctttgatggtcttcctgcttagatgtttatttgtgactgctaggggcgatgttttcttggagataattgaatttttctttgtcaagctcattttgctttcttgtggttgttatggaatgttactggatactggatagttagttcggcctgtaggcgtatcctcctgaacttacctctcattgctgttagttttggcaccaagtttgttcccaattgctatttacggaagttattgcataaagggctgtttggttgtaatcttcccgtaagaataagatgcatgtttttgagttaaaaaagggggggcccggtacccaattcgccctatag

SEQ ID NO: 11 - protein, 486 aa, Zea mays
MAAANLLSRSLLPTPNTIRTSHPTPRSPAVVSFPRRRARLSVCASVSMASPSPPPQPAAAGVPKHCFRRGADGYLYCEGVRVEDAMAAAERSPFYLYSKLQILRNFAAYRDALQGLRSIVGYAVKANNNLPVLRVLRELGCGAVLVSGNELRLALQAGFDPARCIFNGNGKTLEDLKLAAESGVFVNVDSEFDLENIVRAARATGKKVPVLLRINPDVDPQVHPYVATGNKTSKFGIRNEKLQWFLNSIKSYSNEIKLVGVHCHLGSTITKVDIFRDAAVLMVNYVDEIRAQGFKLEYLNIGGGLGIDYHHTDAVLPTPMDLINTVRELVLSQDLTLIIEPGRSLIANTCCFVNRVTGVKSNGTKNFIVVDGSMAELIRPSLYGAYQHIELVSPPTPGAEVATFDIVGPVCESADFLGKDRELPTPDEGAGLVVHDAGAYCMSMASTYNLKLRPPEYWVEEDGSIVKIRHEEKLDDYMKFFDGLPA

SEQ ID NO: 12 - DNA, 1116 bp, Oryza sativa
cttacacggagtgtttgtaaacatagacagtgaatttgatttggagaatattgtcactgctgcgagagttgctgggaagaaagtccctgttttgctcaggataaacccagatgtggatccacaggtccatccttatgttgcgactggaaacaaaacctccaaatttggtatccgtaatgagaaactacaatggttcttagactctatcaagtcatactcaaatgatatcacactggtgggtgttcattgtcatctgggatctaccattacaaaggtcgatatatttagagatgcggcaggtcttatggtgaattatgttgatgaaattcgagcacaaggttttgaactggaatatctcaatattggcggtggcctgggcatagwttatcaccacacggatgcagtcttgcctacacctatgggacctcatcaacactgtgccgaagaattagttctgtcacgagatcttacactcatcattgaacctgggagatccctcatagctaacacttgctgcttcgtcaatagggtcactggtgttaaatctaatggtacaaagaatttcattgtagttgatggcagcatggcagagcttatcagaccaagtctatatggagcataccagcatatcgaactggtttctccttccccagatgcagaagtagcaacattcgatattgttggaccagtttgtgaatctgcagatttccttggcaaagacagggaacttccaacacctgataagggagctggtttggtggttcatgacgcaggagcctactgcatgagcatggcttcaacctacaacttgaagttgcgaccacctgaatattgggtagaagatgatgggtccattgctaagattcggcgtggagagtcatttgatgactacatgaagttctttgataatctctctgcctaactcgttttcctgcaattgtaataagatttttctcttgttatgtgtggctgtatcaggattcggattgatagcgcagtacagtttgctgtagaatcggtatttttttttattgtactgtgatgtcggtaccttattttatccaaagatttttggcaaattttgctacaggacacttaaaaaaaaaaaaaaaaaaa

SEQ ID NO: 13 - protein, 306 aa, Oryza sativa
  UNSURE (128): Xaa = any amino acid
LHGVFVNIDSEFDLENIVTAARVAGKKVPVLLRINPDVDPQVHPYVATGNKTSKFGIRNEKLQWFLDSIKSYSNDITLVGVHCHLGSTITKVDIFRDAAGLMVNYVDEIRAQGFELEYLNIGGGLGIXYHHTDAVLPTPMGPHQHCAEELVLSRDLTLIIEPGRSLIANTCCFVNRVTGVKSNGTKNFIVVDGSMAELIRPSLYGAYQHIELVSPSPDAEVATFDIVGPVCESADFLGKDRELPTPDKGAGLVVHDAGAYCMSMASTYNLKLRPPEYWVEDDGSIAKIRRGESFDDYMKFFDNLSA

SEQ ID NO: 14 - DNA, 968 bp, Glycine max
gttgccactgggaataagaactctaaatttggcattagaaatgagaagctgcagtgctttttagatgcagtgaaggaacatcctaatgagctcaaacttgtaggggcccactgccatcttggttcaacaattaccaaggttgacattttcagggatgcagccaccattatgatcaactacattgaccaaatccgagatcagggttttgaagttgattacttaaatattggtggaggacttgggatagattattatcattctggtgccatccttcctacacctagagatctcattgacactgtacgagatcttgttatttcacgtggtcttaatctcatcattgaaccaggaagatcactcattgcaaacacgtgttgcttagttaaccgggtgacaggtgttaaaactaatggatctaaaaacttcattgtaattgatggaagtatggctgaacttatccgccctagtctttatgatgcttaccagcatatagagctggtttcccctgccccgtcaaatgctgaaacagaaacttttgatgtggttggccctgtctgtgagtctgcagatttcttaggaaaaggaagagaacttcctactccagccaagggtactggtttggttgttcatgatgctggtgcttattgcatgagcatggcatcaacctacaatctaaagatgcggcctcctgagtattgggttgaagatgatggatcagtgagcaaaataagacatggagagacttttgaagaccacattcggttttttgaggggctttgagctaataatttatcttgtaggaaagaaggctggagaattgttatgtacttggagtttgaatctttcctcgtcaatgaatgcatgactcttgtagttctgtttcttccgttctaattgaatgttgactcccatgacaggaacagagaataaagttgatttcagttagatttaaaaaaaaaaaaaaaaaa

SEQ ID NO: 15 - protein, 259 aa, Glycine max
VATGNKNSKFGIRNEKLQCFLDAVKEHPNELKLVGAHCHLGSTITKVDIFRDAATIMINYIDQIRDQGFEVDYLNIGGGLGIDYYHSGAILPTPRDLIDTVRDLVISRGLNLIIEPGRSLIANTCCLVNRVTGVKTNGSKNFIVIDGSMAELIRPSLYDAYQHIELVSPAPSNAETETFDVVGPVCESADFLGKGRELPTPAKGTGLVVHDAGAYCMSMASTYNLKMRPPEYWVEDDGSVSKIRHGETFEDHIRFFEGL

SEQ ID NO: 16 - DNA, 676 bp, Triticum aestivum
  unsure (373): n = a, c, g or t
  unsure (406): n = a, c, g or t
  unsure (430): n = a, c, g or t
  unsure (433): n = a, c, g or t
  unsure (455): n = a, c, g or t
  unsure (494): n = a, c, g or t
  unsure (504): n = a, c, g or t
  unsure (553): n = a, c, g or t
  unsure (579): n = a, c, g or t
  unsure (583): n = a, c, g or t
  unsure (595)..(596): n = a, c, g or t
  unsure (620): n = a, c, g or t
  unsure (639): n = a, c, g or t
  unsure (644): n = a, c, g or t
  unsure (650): n = a, c, g or t
  unsure (661): n = a, c, g or t
  unsure (673): n = a, c, g or t
tttgagttggagtacctgaatattggaggtggtttggggatagactaccaccacactggtgcagtcttgcctacacctatggatcttatcaacactgtccgggaattggtcctctcacgggatcttactctcattattgaacctggaagatccctgatcgccaatacttgctgcttcgtcaataaggtcactggtgtaaaatcgaatggcacgaagaatttcattgtagttgatggcagcatggccgagctcatcaggcctagtctatatggagcatatcagcatatagaactagttctccctctccaaggtgcagaagtagcaaccttccgatattgttggggccagtctgcgaatctgcagattccttggnaaagacaaggagttccaacacctgacaaggganctggtttgggtgtccacgacgcanganctactgcatgagcatggcttcnacctacaacctgaagatgaggcaaccgagtattgggtanaggacatggnccatgtaagataagcacggggaaacattgacgacacatgagtcttgatngctccgccaggcctttactggttggnaacnagcttcattgtnnccaccgtggaatctgggaacatcntgttgtagtggcaccacanagggnttttgngacaatcacantagatgagattntgg

SEQ ID NO: 17 - protein, 73 aa, Triticum aestivum
PTPMDLINTVRELVLSRDLTLIIEPGRSLIANTCCFVNKVTGVKSNGTKNFIVVDGSMAELIRPSLYGAYQHI

SEQ ID NO: 18 - DNA, 544 bp, Glycine max
  unsure (465): n = a, c, g or t
  unsure (524): n = a, c, g or t
  unsure (537): n = a, c, g or t
ttgcaacacacattgtcttgtcggcaaaatcttccaccaacaacacacagccatggcaggctcaaacattctttctcactctccttcccttcccaaaacctacagccactccttaaaccaaaacgcgttatcccaaaagcttttttttctgcccctcaaattcaaagccaccacaaaaccacgtgctctcagagcggttctctcgcagaacgctgtcaaaacctcggtggaggacacaaagaacgctcattttcagcactgtttcaccaaatccgaagatgggtatctgtactgtgagggcctcaaggtgcatgacatcatggaatctgttgagagaagacctttctatttgtacagcaagccccagataactaggaatgttgaagcctacaaggatgcattggaagggttgaactccataattggttatgccattaaggccaataataacttgaagattttggnacatttgaggcacttgggttgtggtgctgtgcttgttagtgggaatgagctgaagttgntcttcgagctggntttgttc

SEQ ID NO: 19 - protein, 62 aa, Glycine max
  Unsure (44): Xaa = any amino acid
RRPFYLYSKPQITRNVEAYKDALEGLNSIIGYAIKANNNLKILXHLRHLGCGAVLVSGNELK

SEQ ID NO: 20 - protein, 371 aa, Pseudomonas aeruginosa
MKRVGLIGWRGMVGSVLIQRMLEERDFDLIEPVFFTTSNVGAQAPEVDKDIAPLKDAYSIDELKTLDVILTCQGGDYTSEVFPKLREAGWQGYWIDAASSLRMEDDAVIVLDPVNRKVIDQALDAGTRNYIGGNCTVSLMLMALGGLFDAGLVEWMSAMTYQAASGAGAQNMRDLLKQMGAAHASVADDLANPASAILDIDRKVAETLRSEAFPTEHFGAPLGGSLIPWIDKELSQRRQSREEWKAQAETNKILARFKNPIPVDGICVRVGAMRCHSQALTIKLNKDVPLTDIEGLIRQHNPWVKLVPNHREVSVRELTPAAVTGTLSVPVGRLRKLNMVSQYLGAFTVGDQLLWGAAEPLRRMLRILLER

SEQ ID NO: 21 - DNA, 788 bp, Zea mays
cgacaacatcgcccccgccatcctcggcggcttcgtcctcgtccgcagctacgacccctttcacctcgtcccgctttccttcccgccagcgctccgcctccacttcgtcctggtcacccccgacttcgaggcgcccacgagcaagatgcgcgccgcgctgcccaggcaggtcgacgtccagcagcacgtgcgcaactccagccaggcagcggcgctcgtggcggcggtgctgcagggggacgcgggcctcatcggctccgcgatgtcgtccgacggcatcgtggagcccaccagggcacccctcatacctggcatggcggccgtaaaggcggcggccctgcaagctggagcgctgggctgcacaattagcggcgcgggccccacagtggtggccgtcatccaaggggaggaaaggggggaggaggttgcccgcaagatggtggacgcgttctggagcgcaggcaagctcaaggcgacagcaaccgtcgcgcagctcgatacccttggtgccagggtcatcgccacgtcatccttgaactagcaaaagattcggaaagtggtactgcaattgtatcaccaaacaaggaagaatgaaggggaaccccatggatttgtatgttttctcttctttcttgcatctttaggtggttaattggctttggaataaatgagatggaggacatcgctagaacaattctgttccgtgggctgtaatttcaatttgggctggtttctttatcatgccatggataattatgaataaatttgaggtagtttgttaaaaaaaa

SEQ ID NO: 22 - protein, 179 aa, Zea mays
DNIAPAILGGFVLVRSYDPFHLVPLSFPPALRLHFVLVTPDFEAPTSKMRAALPRQVDVQQHVRNSSQAAALVAAVLQGDAGLIGSAMSSDGIVEPTRAPLIPGMAAVKAAALQAGALGCTISGAGPTVVAVIQGEERGEEVARKMVDAFWSAGKLKATATVAQLDTLGARVIATSSLN

SEQ ID NO: 23 - DNA, 601 bp, Oryza sativa
  unsure (433): n = a, c, g or t
  unsure (600): n = a, c, g or t
gtcgccgccatcgctgcccttcgcgccctcgatgtcaagtcccacgccgtctccatccacctcaccaagggcctccccctcggctccggcctcggctcctccgccgcctccgccgccgccgctgccaaggccgttgacgccctcttcggctccctcctacaccaagatgacctcgtcctcgcgggcctcgagtccgagaaagccgtcagtggcttccacgccgacaacatcgccccggccatcctcggcggcttcgtcctcgtccgcagctacgaccccttccacctcatcccgctctcctccccacctgccctccgcctccacttcgtcctcgtcacgcccgacttcgaggcgcccaccaagcaagatgcgtgccgcgctgcccaaacaggtggccgtccaccaagcacgtccgcaactccagccaagcggncgcgcttgtcgccgctgtgctgcaaggggacgccaccctcatcggctccgcaatgtcctccgacggcatcgtggagccaacaaggcgccgctgattctggatggctgcggtcaaaggcgccggcttggaactgggggaattggctgcacatcagtggagaaggcaant

SEQ ID NO: 24 - protein, 82 aa, Oryza sativa
  UNSURE (56)..(57): Xaa = any amino acid
VSIHLTKGLPLGSGLGSSAASAAAAAKAVDALFGSLLHQDDLVLAGLESEKAVSGXXHADNIAPAILGGFVLVRSYDPFHLI

SEQ ID NO: 25 - DNA, 1543 bp, Glycine max
gaagagagacaaaccagcaagagtggagatggcgacgtcgacgtgcttcctgtgtccgtctacggcgagtttgaaaggcagggccagattcagaatcagaatcagatgcagcagcagcgtgtcggtcaatattcgaagggagcccgaacctgtaacgacgctggtgaaagcgtttgctcccgccacggtggcgaatctaggtccaggcttcgacttcctaggctgcgccgtggacggactcggagacattgtgtcggtgaaggttgacccacaggttcaccctggcgagatatgcatatccgacatcagcggccacgccccaaacaagctcagcaaaaaccctctctggaactgcgccggcatcgccgccattgaagtcatgaaaatgctctccattcgatccgtcggcctctccctctccctggagaagggcctgcctttgggaagcggtctgggatccagcgccgccagcgccgccgcggccgccgtggcggtgaacgagctgtttgggaagaaattaagcgtggaggagctggttctggcatcactgaaatcggaagagaaggtgtcggggtatcacgcggacaacgtggcgccatcgataatggggggttttgtgctgatcgggagctactcgccgctggagttgatgccgttgaagtttccggcagagaaggagctgtatttcgtgctggtgacgcctgagttcgaggccccgacgaagaagatgcgggcagcgctgcctacggagatcgggatgccgcaccacgtgtggaactgcagccaggcaggtgctctggtggcgtcggtgctgcagggcgacgtggttgggttggggaaggcattgtcctctgacaagatcgttgagccaaggcgtgcccccttgattcctggcatggaggctgtcaagagggctgccattcaggccggtgcttttggctgtaccatcagcggcgccggccctaccgccgtcgccgtcattgacgacgagcaaactggacacctcattgccaaacacatgattgacgcttttctccatgttggcaatttgaaggcttctgcaaatgtcaagcagcttgatcgccttggtgctagacgcattccaaattgaaccttctcttctctatctctatgagaggcttgtagatttcaagaaccggatttcttccaacttgctcgtaacactctaagtgctgaccggtcacatgtatttgaaatttgatctgatcaatgaagcagcattctagtgtggaggtctgaataacaagagaaacattaaacccaagctgggagctctgtttgggtggtggaaatttaaatagatgaataattatgaaagacctagatcaggtcagtgttatggtgaactctgaagcatgttttagattttctttgctttgtttttatcatatttttatcttgctacttgagttgacaaagctcaaaaagaagtcatttttagtattttcttgtttcattatgctagttaatcttagcttttgaatagcatgtattgttccttaaaaaaaaaaaaaaaaaaaaa

SEQ ID NO: 26 - protein, 483 aa, Glycine max
MATSTCFLCPSTASLKGRARFRIRIRCSSSVSVNIRREPEPVTTLVKAFAPATVANLGPGFDFLGCAVDGLGDIVSVKVDPQVHPGEICISDISGHAPNKLSKNPLWNCAGIAAIEVMKMLSIRSVGLSLSLEKGLPLGSGLGSSAASAAAAAVAVNELFGKKLSVEELVLASLKSEEKVSGYHADNVAPSIMGGFVLIGSYSPLELMPLKFPAEKELYFVLVTPEFEAPTKKMRAALPTEIGMPHHVWNCSQAGALVASVLQGDVVGLGKALSSDKIVEPRRAPLIPGMEAVKRAAIQAGAFGCTISGAGPTAVAVIDDEQTGHLIAKHMIDAFLHVGNLKASANVKQLDRLGARRIPNTFSSLSLEACRFQEPDFFQLARNTLSADRSHVFEISDQSSILVWRSEQEKHTQAGSSVWVVEIIDELKTIRSVLWTLKHVLDFLCFVFIIFLSCYLSQSSKRSHFYFLVSLCLILAFEHVLFL

SEQ ID NO: 27 - DNA, 438 bp, Triticum aestivum
  unsure (271): n = a, c, g or t
  unsure (421): n = a, c, g or t
  unsure (425): n = a, c, g or t
ctcgagtcggagaaggccgtcagcggcttccacgccgacaacatcgcccccgccatcctcggcggcttcgtcctcgtccgcagctacgacccctttcacctcgtcccgctttccttcccgccagcgctccgcctccacttcgtcctggtcacccccgacttcgaggcgcccacgagcaagatgcgcgccgcgctgcccaggcaggtcgacgtccagcagcacgtgcgcaactccagccaggcagcggcgctccgtggcggcggtgctgcanggggacgccgggctcatcggtccgcgatttctccgacgggcatcgtggacccaccaaggaaccctcatacctggcatggcggccgtaaaggcggcggcctgcaactggacgctgggtgcacattaacgggcgggcccacatggtggctcncagngaagagaggggag

SEQ ID NO: 28 - protein, 84 aa, Triticum aestivum
LESEKAVSGFHADNIAPAILGGFVLVRSYDPFHLVPLSFPPALRLHFVLVTPDFEAPTSKMRAALPRQVDVQQHVRNSSQAAAL

SEQ ID NO: 29 - protein, 300 aa, Methanococcus jannashii
MREIMKVRVKAPCTSANLGVGFDVFGLCLKEPYDVIEVEAIDDKEIIIEVDDKNIPTDPDKNVAGIVAKKMIDDFNIGKGVKITIKKGVKAGSGLGSSAASSAGTAYAINELFKLNLDKLKLVDYASYGELASSGAKHADNVAPAIFGGFTMVTNYEPLEVLHIPIDFKLDILIAIPNISINTKEAREILPKAVGLKDLVNNVGKACGMVYALYNKDKSLFGRYMMSDKVIEPVRGKLIPNYFKIKEEVKDKVYGITISGSGPSIIAFPKEEFIDEVENILRDYYENTIRTEVGKGVEVV

SEQ ID NO: 30 - DNA, 1362 bp, Glycine max
actttgtagttcgtagatagccgatgtgcttgtcttagtgtgtcagtcattcctgttcctcaagtcaagctttgtagtgagcagatataatggctgttgaaaggtccggaattgccaaagatgttacggaattgattggtaaaaccccattagtatatctaaataaacttgcggatggttgtgttgcccgggttgctgctaaactggagttgatggagccatgctctagtgtgaaggacaggattgggtatagtatgattgctgatgcagaagagaagggacttatcacacctggaaagagtgtcctcattgagccaacaagtggtaatactggcattggattagccttcatggcagcagccaggggttacaagctcataattacaatgcctgcttctatgagtcttgagagaagaatcattctattagcttttggagctgagttggttctgacagatcctgctaagggaatgaaaggtgctgttcagaaggctgaagagatattggctaagacgcccaatgcctacatacttcaacaatttgaaaaccctgccaatcccaaggttcattatgaaaccactggtccagagatatggaaaggctccgatgggaaaattgatgcatttgtttctgggataggcactggtggtacaataacaggtgctggaaaatatcttaaagagcagaatccgaatataaagctgattggtgtggaaccagttgaaagtccagtgctctcaggaggaaagcctggtccacacaagattcaagggattggtgctggttttatccctggtgtcttggaagtcaatcttcttgatgaagttgttcaaatatcaagtgatgaagcaatagaaactgcaaagcttcttgcgcttaaagaaggcctatttgtgggaatatcttccggagctgcagctgctgctgcttttcagattgcaaaaagaccagaaaatgccgggaagcttattgttgccgtttttcccagcttcggggagaggtacctgtcctccgtgctatttgagtcagtgagacgcgaagctgaaagcatgacttttgagccctgaattcccgtttaaggctctcactactgaattttcttgttacttgtaccaggctttaactagattgttagagtactactgtttgtgactctgactctaaaataaaacttgctccaaaagactagtttttcttgatgcccctggagcgataattttgtgcctgcaacattaaaaagtattcaaagttgcttataagtaacatgtttcatcttttgttgttgttgagacgaacacggatgaggtcataatactatgtttctgatttcctttggtagggaaaaaaaaaaaaaaaaaaaaa

SEQ ID NO: 31 - protein, 325 aa, Glycine max
MAVERSGIAKDVTELIGKTPLVYLNKLADGCVARVAAKLELMEPCSSVKDRIGYSMIADAEEKGLITPGKSVLIEPTSGNTGIGLAFMAAARGYKLIITMPASMSLERRIILLAFGAELVLTDPAKGMKGAVQKAEEILAKTPNAYILQQFENPANPKVHYETTGPEIWKGSDGKIDAFVSGIGTGGTITGAGKYLKEQNPNIKLIGVEPVESPVLSGGKPGPHKIQGIGAGFIPGVLEVNLLDEVVQISSDEAIETAKLLALKEGLFVGISSGAAAAAAFQIAKRPENAGKLIVAVFPSFGERYLSSVLFESVRREAESMTFEP

SEQ ID NO: 32 - protein, 325 aa, Citrullus lanatus
MADAKSTIAKDVTELIGNTPLVYLNRVVDGCVARVAAKLEMMEPCSSVKDRIGYSMISDAENKGLITPGESVLIEPTSGNTGIGLAFIAAAKGYRLIICMPASMSLERRTILRAFGAELVLTDPARGMKGAVQKAEEIKAKTPNSYILQQFENPANPKIHYETTGPEIWRGSGGKIDALVSGIGTGGTVTGAGKYLKEQNPNIKLYGVEPVESAILSGGKPGPHKIQGIGAGFIPGVLDVNLLDEVIQVSSEESIETAKLLALKEGLLVGISSGAAAAAAIRIAKRPENAGKLIVAVFPSFGERYLSTVLFESVKRETENMVFEP

SEQ ID NO: 33 - DNA, 789 bp, Zea mays
atagcgcattctcatggtgctcttgttttggttgacaacagcatcatgtctccagtgctctcccgtcctatagaactgggagctgatatcgtgatgcactcggctaccaaatttatagcgggacatagtgatcttatggctggaattcttgcagtgaagggtgagagtttggctaaagaggtagggtttctgcaaaatgctgaagggtcgggtctggcaccttttgactgctggctttgcttgaggggaatcaaaaccatggctctgcgggtggagaaacaacaggctaatgcccagaagattgctgaattcctggcgtctcacccgagggtcaagcaagtaaactacgctgggcttcctgaccatcctgggcgagctttacactattcccaggcaaagggagcgggctctgttctcagttttctcaccggctcactggccctctcaaagcacgtcgtggagaccaccaagtacttcagcgtaacagtcagcttcgggagcgtgaagtccctcatcagcctgccgtgcttcatgtcccacgcatcaatccctgcctcggtccgcgaggagcgtggcctaaccgacgacctcgtccggatatcggtcggcatcgaggatgtcgaggacctcatcgccgatctggaccgcgcgctcagaactggcccggtgtagacatcgccgatccttaggtcatgtcaagctatcttttgatgattcattggttgactgcttgcgtgatgataataatgggaatgttgcttggataaaaaaaaaaaaaaaaaaactcga

SEQ ID NO: 34 - protein, 223 aa, Zea mays
IAHSHGALVLVDNSIMSPVLSRPIELGADIVMHSATKFIAGHSDLMAGILAVKGESLAKEVGFLQNAEGSGLAPFDCWLCLRGIKTMALRVEKQQANAQKIAEFLASHPRVKQVNYAGLPDHPGRALHYSQAKGAGSVLSFLTGSLALSKHVVETTKYFSVTVSFGSVKSLISLPCFMSHASIPASVREERGLTDDLVRISVGIEDVEDLIADLDRALRTGPV

SEQ ID NO: 35 - DNA, 547 bp, Oryza sativa
  unsure (260): n = a, c, g or t
  unsure (306): n = a, c, g or t
  unsure (376): n = a, c, g or t
  unsure (383): n = a, c, g or t
  unsure (404): n = a, c, g or t
  unsure (432): n = a, c, g or t
  unsure (446): n = a, c, g or t
  unsure (455)..(456): n = a, c, g or t
  unsure (509): n = a, c, g or t
  unsure (514): n = a, c, g or t
  unsure (522): n = a, c, g or t
  unsure (537): n = a, c, g or t
gccttatggctaagcttgagaaggcggatcaggcattctgcttcaccagtgggatggcagcactagctgcagtaacacacctccttaagtctggacaagaaatagttgctggagaggacatatatggtggctcagaccgtctgctctcacaagttgccccgagacatgggattgtagtaaaacgaattgatacaaccaaaattagtgaggtaacttctgcaattggggccttggactaaactaagtatggctttgaaaancccaccatccccgtcctacaaattactggatataaagaaaatagcnagagatagtcattacaatggggctccttgttttaagtagacaacagcacatgtctccctgtgctctcccngtcctcntaaaactttgggccaaatatnggtttgcaccccaagcaaccaatttatnctgggcatagcgtncttatggcnnggatccttgccgggaaggggtgaaagcacttggctaaagagatgcattcctcnaaaanctgaaggntaagtttggacattngatgccggtt

SEQ ID NO: 36 - protein, 75 aa, Oryza sativa
LMAKLEKADQAFCFTSGMAALAAVTHLLKSGQEIVAGEDIYGGSDRLLSQVAPRHGIVVKRIDTTKISEVTSAIG

SEQ ID NO: 37 - DNA, 1733 bp, Glycine max
caaagacggcattgaagttgaacaatccatcactaacacaagcgcagacaacaacataaccctgctccaaacacatcaatttcaataatgttttcttctgcaatttctcagaagcccttccttcagtccctcgtcattgatcgttacgctcagagcacaactgctgcaaccaggtgggagtgcttggggtttaacaagtcagaaaatttcagtaccaagagagtgttgcgtgcagaggggttcaagttgaattgcttggttgaaaatagagagatggaagtggagtcatcatcatcatctttggtggatgatgctgccatgagcttaagtgaagaggatttaggggagcctagtatttcaacaatggtgatgaatttcgagagtaagtttgatccttttggagcaattagtaccccgctttaccaaacggctacttttaagcagccttctgcaatagaaaatggtccctatgactataccagaagtggaaatcctactcgtgatgctttagaaagtttactagcaaagcttgataaagcagatagagccctgtgcttcaccagtggaatggctgctttgagtgctgttgttcgtcttgttggaactggtgaggaaattgtcaccggagatgatgtatatggtggctcagataggttgctgtctcaagtagttccaaggactggaattgtggtgaaacgggtaaatacatgtgatctagatgaggttgctgctgccattggactcaggactaagcttgtgtggcttgagagtccaaccaatcctcggcttcaaatttctgatattcgaaaaatatcagagatggctcattcacatggtgctcttgtgttagtggacaatagtataatgtcacctgtgttgtctcagccattggaacttggagcagatattgtcatgcactcagctacaaaatttattgctggacatagtgacattatggctggtgtgcttgctgtgaagggtgaaaagttgggaaaggaaatgtatttcttgcaaaatgcagagggttcaggcttagcaccatttgactgttggctttgtttgcgaggaatcaagacaatggccctgcgaattgaaaagcaacaggataacgcacagaagattgcagagttccttgcctcccatcctcgagtgaaggaagtgaattatgctggcttgcctggtcatcctggtcgtgatttacactattctcaggcaaagggtgcaggatctgtgcttagcttcttgactggttcattggcactttcaaagcatattgttgaaactaccaaatacttcagtataaccgtcagctttgggagtgtgaagtccctcattagcatgccatgctttatgtcacatgcaagcatacctgctgcagttcgcgaggccagaggtttaactgaagatcttgtacgaatatctgtgggaattgaggatgtgaatgatctcattgctgatcttggcaatgcacttagaactggacctctttaatgtcttctccacccccccacccaaaaagaaaaaaattcatccttaagaagttggattagcatgttgaggatttgggagcattgctatcctgtctttggattcttgagagtggaaacttgaagtgttgcttatgtgcatgtaataaaatcaatatttcctgtaattttgttgtaacaattgttatccttaccttgcaatatcatgtcatacaagttactattgaaaaaaaaaaaaaaaaaaaa

SEQ ID NO: 38 - protein, 467 aa, Glycine max
MFSSAISQKPFLQSLVIDRYAQSTTAATRWECLGFNKSENFSTKRVLRAEGFKLNCLVENREMEVESSSSSLVDDAAMSLSEEDLGEPSISTMVMNFESKFDPFGAISTPLYQTATFKQPSAIENGPYDYTRSGNPTRDALESLLAKLDKADRALCFTSGMAALSAVVRLVGTGEEIVTGDDVYGGSDRLLSQVVPRTGIVVKRVNTCDLDEVAAAIGLRTKLVWLESPTNPRLQISDIRKISEMAHSHGALVLVDNSIMSPVLSQPLELGADIVMHSATKFIAGHSDIMAGVLAVKGEKLGKEMYFLQNAEGSGLAPFDCWLCLRGIKTMALRIEKQQDNAQKIAEFLASHPRVKEVNYAGLPGHPGRDLHYSQAKGAGSVLSFLTGSLALSKHIVETTKYFSITVSFGSVKSLISMPCFMSHASIPAAVREARGLTEDLVRISVGIEDVNDLIADLGNALRTGPL

SEQ ID NO: 39 - DNA, 637 bp, Triticum aestivum
  unsure (400): n = a, c, g or t
  unsure (417): n = a, c, g or t
  unsure (486): n = a, c, g or t
  unsure (492)..(493): n = a, c, g or t
  unsure (505): n = a, c, g or t
  unsure (518): n = a, c, g or t
  unsure (524): n = a, c, g or t
  unsure (530): n = a, c, g or t
  unsure (532): n = a, c, g or t
  unsure (557): n = a, c, g or t
  unsure (563): n = a, c, g or t
  unsure (581): n = a, c, g or t
  unsure (591): n = a, c, g or t
  unsure (596): n = a, c, g or t
  unsure (617): n = a, c, g or t
agcgtggccacgatactgaccagcttcgagaactcgttcgacaagtatggggctctcagcacgccgctgtaccagacggccaccttcaagcagccttcagcaaccgttaatggagcttatgattatactagaagtggcaaccctactcgtgatgttctccagagccttatggctaagctcgagaaggcagaccaagcattctgcttcactagtgggatggcatcactgggctgcagtaacacacctccttcaggctggacaagaaatagttgctggagaggacatatatggtggtctgatcgtctgctctcacaagttgtcccaagaaatggaattgtagtaaaacgggtcgatacaactaaaattaacgacgtgactgctgcatcggacccttgactanactagtttggttgaaanccacaatcctcgtcaacaattactgtataagaaatctcagggatactcatccatggggactggtttggnggcaannttcatgtcccanggctacctggccnataaantggggnantatgggagcatcagtacaaattatnctggcnatgtctaggtggatctcntaaggggaanttggnaggattcttcaaaacctagtnggttgacttatgtggttgtt

SEQ ID NO: 40 - protein, 131 aa, Triticum aestivum
  UNSURE (77): Xaa = any amino acid
  UNSURE (99): Xaa = any amino acid
SVATILTSFENSFDKYGALSTPLYQTATFKQPSATVNGAYDYTRSGNPTRDVLQSLMAKLEKADQAFCFTSGMASLXAVTHLLQAGQEIVAGEDIYGGXDRLLSQVVPRNGIVVKRVDTTKINDVTAASDP

SEQ ID NO: 41 - protein, 464 aa, Arabidopsis thaliana
MTSSLSLHSSFVPSFADLSDRGLISKNSPTSVSISKVPTWEKKQISNRNSFKLNCVMEKSVDGQTHSTVNNTTDSLNTMNIKEEASVSTLLVNLDNKFDPFDAMSTPLYQTATFKQPSAIENGPYDYTRSGNPTRDALESLLAKLDKADRAFCFTSGMAALSAVTHLIKNGEEIVAGDDVYGGSDRLLSQVVPRSGVVVKRVNTTKLDEVAAAIGPQTKLVWLESPTNPRQQISDIRKISEMAHAQGALVLVDNSIMSPVLSRPLELGADIVMHSATKFIAGHSDVMAGVLAVKGEKLAKEVYFLQNSEGSGLAPFDCWLCLRGIKTMALRIEKQQENARKIAMYLSSHPRVKKVYYAGLPDHPGHHLHFSQAKGAGSVFSFITGSVALSKHLVETTKYFSIAVSFGSVKSLISMPCFMSHASIPAEVREARGLTEDLVRISAGIEDVDDLISDLDIAFKTFPL

SEQ ID NO: 42 - DNA, 1113 bp, Zea mays
gccgtccaggacctcgcggcccctggggcgttcgacggcgtcgacatcgcgctattcagcgccggcgggagcgtcagccggaagtatgggcccgcggccgtcgccagcggcgccgtagttgtcgacaacagctccgcgttccggatggagcccgaggtgccgctcgtcatccccgaggtcaaccccgaggccatggcgaacgtccgcctcgggcagggggcgattgtggcaaatccgaattgctcgaccatcatctgcctcatggctgccacgccgctccatcgccacgctaaggtgttaaggatggttgtcagcacataccaagcagcaagtggtgcgggtgctgcggcaatggaagaactcaagctgcagactcaggaggtcttggaagggaaggcgccaacatgcaacattttcaaacagcagtatgcttttaatatattctcacacaatgcaccagttcttgagaatgggtataacgaggaggaaatgaaaatggtgaaggagaccaggaaaatttggaatgacaaggaggtgaaagtaactgcgacttgcatacgggttcctgtgatgcgcgcacatgctgaaagtgtcaatctacagtttgaaaagccacttgatgaggatactgcaagagaaattttgagagcagctcctggtgttaccattattgatgaccgagcttccaatcgctttcctacacctctggaggtatcagacaaagatgacgtagcagtgggtaggattcgtcaggacttgtccctggatggtaaccgagggttggacatatttgtgtgtggtgatcagatacgtaaaggcgccgcactcaatgccgttcagattgctgaaatgctgctgaagtgaatgtgacctaaccctcttgtccctccctccctgtccctaattgctctgatcaaatgctggactgtactctgattagtttgtcctcaattttggtcgcctgttctgtattctgccgtgctagtgcaataattgtgttatgggcttgagttatctgctgtacgcataagtgggctcctaaactgggaaataatgggccgtccttattcagcattccggtttatatcttgttcaaaaaaaaaaaaaaaaata

SEQ ID NO: 43 - protein, 287 aa, Zea mays
AVQDLAAPGAFDGVDIALFSAGGSVSRKYGPAAVASGAVVVDNSSAFRMEPEVPLVIPEVNPEAMANVRLGQGAIVANPNCSTIICLMAATPLHRHAKVLRMVVSTYQAASGAGAAAMEELKLQTQEVLEGKAPTCNIFKQQYAFNIFSHNAPVLENGYNEEEMKMVKETRKIWNDKEVKVTATCIRVPVMRAHAESVNLQFEKPLDEDTAREILRAAPGVTIIDDRASNRFPTPLEVSDKDDVAVGRIRQDLSLDGNRGLDIFVCGDQIRKGAALNAVQIAEMLLK

SEQ ID NO: 44 - DNA, 1402 bp, Oryza sativa
gcccaactcccaaaaccctagaaccgcgccgccacaatgcaggccgccgccgccgccgtccaccgcccgcacctcctcggcgcctaccccggcggtggccgcgcgcgccgcccgtcgtccaccgtgcggatggcgcttcgggaggacgggccgtcggtggcgatcgtgggcgcgacgggcgccgtcggccaggagttcctccgcgtcatctcctcccggggcttcccctaccggagcctccgcctcctcgccagcgagcgctccgcggggaagcgcctcccgttcgagggccaggagtacaccgtccaggacctcgccgcgccgggcgcgttcgacggggtggacatcgcgctcttcagcgccggcggcggggtcagccgcgcccacgctcccgcggccgtcgccagcggcgccgtcgtcgtggacaacagctccgccttccggatggaccccgaggtgccgctcgtcatccccgaggtcaatcccgaggccatggcgcacgtccggctgggaaagggggctattgtggccaacccgaactgttccaccatcatctgcctcatggctgccacacctctgcaccgccacgccaaggtggtaaggatggttgtcagcacttaccaagcagcaagtggtgctggggctgcggccatggaagaactcaaacttcaaactcaagaggtcttggcggggaaagcaccaacatgcaacattttcagtcagcagtatgcttttaatatattttcacataatgcaccaattgttgaaaatgggtacaatgaggaggagatgaagatggtgaaggagaccagaaaaatctggaatgataaagatgtgaaggtaactgcaacctgcatacgagttcctgtgatgcgtgcacatgctgaaagtgtgaatctacagtttgaaaagccacttgatgaggatactgcaagggaaatcttgagggcagctgaaggtgttaccattattgatgaccgtgcttccaatcgcttccccacacctcttgaggtatcggataaagatgatgtagcagtgggtagaattcgtcaggatttgtcgcaagatgataacaaagggctggacatatttgtttgtggagatcaaatacgtaaaggtgctgcactcaatgctgtgcagattgctgaaatgctactcaagtgattttcttttctgtacctttctctccttgcccctctttgctctagtcattgtttgacggatgtactctggttagtatgagatcaattttgatcatcttttgtaatctatattcctagtgaaataaatgtaaaacggttttgctctatcttctgcacaagtgtagaagaaatctgaaattgggaaattggagtgtggcccttgttcaaaaaaaaaaaaaaaaaaaaaaaaaaaaaaaaaa

SEQ ID NO: 45 - protein, 375 aa, Oryza sativa
MQAAAAAVHRPHLLGAYPGGGRARRPSSTVRMALREDGPSVAIVGATGAVGQEFLRVISSRGFPYRSLRLLASERSAGKRLPFEGQEYTVQDLAAPGAFDGVDIALFSAGGGVSRAHAPAAVASGAVVVDNSSAFRMDPEVPLVIPEVNPEAMAHVRLGKGAIVANPNCSTIICLMAATPLHRHAKVVRMVVSTYQAASGAGAAAMEELKLQTQEVLAGKAPTCNIFSQQYAFNIFSHNAPIVENGYNEEEMKMVKETRKIWNDKDVKVTATCIRVPVMRAHAESVNLQFEKPLDEDTAREILRAAEGVTIIDDRASNRFPTPLEVSDKDDVAVGRIRQDLSQDDNKGLDIFVCGDQIRKGAALNAVQIAEMLLK

SEQ ID NO: 46 - DNA, 1391 bp, Glycine max
gcacgagcttcactctctgttttgcgccacaaccacctcttctcgggccccctcccggcccgccccaagcccacctcctcctcctcctccaggatccgaatgtccctccgcgagaacggcccctccatcgccgtcgtgggcgtcaccggcgccgtcggccaggagttcctctccgtcctctccgaccgcgacttcccctaccgctccattcatatgctggcttccaagcgctccgctggccgccgcatcaccttcgaggacagggactacgtcgtccaggagctcacgccggagagcttcgacggtgtcgacatcgcgctcttcagcgccggcggctccatcagcaagcacttcggccccatcgccgtcaatcgtggaacggtcgtggtcgacaacagctccgcgtttcggatgaacgagaaggtgcctttggtaattcccgaagtgaaccccgaagcaatgcaaaacatcaaagccggaacgggaaagggcgcactcattgctaaccctaattgctccaccattatatgcttgatggctgctacccctcttcatcgacgtgccaaggtgttacgtatggttgttagtacctatcaggctgcgagtggtgctggtgctgctgcaatggaagagcttgagctgcaaactcgtgaggtgttggaaggaaaaccacccacttgtaaaatatttaaccgacagtatgcttttaatctattctcacataatgcgtctgttctttcaaatggatataatgaagaagaaatgaaaatggtcaaggagaccaggaaaatctggaatgacaaggatgttaaagtaactgccacatgcatacgagttcccatcatgcgagctcatgctgagagtgtgaatcttcaatttgaaagaccccttgatgaggacactgcaagagatattctgaaaaatgctccaggtgtagtggttattgatgatcgtgaatccaatcattttcctactccactggaagtgtcaaacaaggatgatgttgctgttggtaggattcggcaggacctgtctcaggatgggaatcaagggttggacatctttgtatgtggggatcaaattcgcaagggagctgcacttaacgcaatccagattgctgagatgttgctatgagttctggtttttcaaggatctggtacttaaagattatgcttcttttgaaacagttttgtatgtgctagttgtatgtggttattcatttcttttgtgatgtttaactagtccaagtatcttttcaacgatgtggtagcacactagctggaaacagtttttttaaggtcttggtgcgtaatatctgcaatccttttcaccgggaataacaagcactggttatggcaaaaaaaaaaaaaaaaaaaaaaaaaaaaaaaaaa

SEQ ID NO: 47 - protein, 377 aa, Glycine max
ARASLSVLRHNHLFSGPLPARPKPTSSSSSRIRMSLRENGPSIAVVGVTGAVGQEFLSVLSDRDFPYRSIHMLASKRSAGRRITFEDRDYVVQELTPESFDGVDIALFSAGGSISKHFGPIAVNRGTVVVDNSSAFRMNEKVPLVIPEVNPEAMQNIKAGTGKGALIANPNCSTIICLMAATPLHRRAKVLRMVVSTYQAASGAGAAAMEELELQTREVLEGKPPTCKIFNRQYAFNLFSHNASVLSNGYNEEEMKMVKETRKIWNDKDVKVTATCIRVPIMRAHAESVNLQFERPLDEDTARDILKNAPGVVVIDDRESNHFPTPLEVSNKDDVAVGRIRQDLSQDGNQGLDIFVCGDQIRKGAALNAIQIAEMLL

SEQ ID NO: 48 - DNA, 1470 bp, Glycine max
gcacgaggtctgttttaaaatccaacacttaatctctctcttcgcagcctaaaatcccaatggcttcactctctgttttgcgccacaaccacctcttctcgggccccctcccggcccgccccaagcccacctcctcctcctcctccaggatccgaatgtccctccgcgagaacggcccctccatcgccgtcgtgggcgtcaccggcgccgtcggccaggagttcctctccgtcctctccgaccgcgacttcccctaccgctccattcatatgctggcttccaagcgctccgctggccgccgcatcaccttcgaggacagggactacgtcgtccaggagctcacgccggagagcttcgacggtgtcgacatcgcgctcttcagcgccggcggctccatcagcaagcacttcggccccatcgccgtcaatcgtggaacggtcgtggtcgacaacagctccgcgtttcggatggacgagaaggtgcctttggtaattcccgaagtgaaccccgaagcaatgcaaaacatcaaagccggaacgggaaagggcgcactcattgctaaccctaattgctccaccattagatgcttgaaggctgctacccctcttcatcgacgtgccaaggtgttacgtatggttgttagtacctatcaggctgcgagtggtgctggtgctgctgcaatggaagagcttgagctgcaaactcgtgaggtgttggaaggaaaaccacccacttgtaaaatatttaaccgacagtatgcttttaatctattctcacataatgcgtctgttctttcaaatggatataatgaagaagaaatgaaaatggtcaaggagaccaggaaaatctggaatgacaaggatgttaaagtaactgccacatgcatacgagttcccatcatgcgagctcatgctgagagtgtgaatcttcaatttgaaagaccccttgatgaggacactgcaagagatattctgaaaaatgctccaggtgtagtggttattgatgatcgtgaatccaatcattttcctactccactggaagtgtcaaacaaggatgatgttgctgttggtaggattcggcaggacctgtctcaggatgggaatcaagggttggacatctttgtatgtggggatcaaattcgcaagggagctgcacttaacgcaatccagattgctgagatgttgctatgagttctggtttttcaaggatctggtacttaaagattatgcttcttttgaaacagttttgtatgtgctagttgtatgtggttattcatttcttttgtgatgtttaactagtccaagtatcttttcaacgatgtggtagcacactagctggaaacagtttttttaaggtcttggtgcgtaatatctgcaatccttttcaccgggaataacaagcactggttttggcaaaaaaaaaaaaaaaaaaaaaaaaaaaaaaaaaaaaaaaaaaaaaaaaaaaaaaaaa

SEQ ID NO: 49 - protein, 376 aa, Glycine max
MASLSVLRHNHLFSGPLPARPKPTSSSSSRIRMSLRENGPSIAVVGVTGAVGQEFLSVLSDRDFPYRSIHMLASKRSAGRRITFEDRDYVVQELTPESFDGVDIALFSAGGSISKHFGPIAVNRGTVVVDNSSAFRMDEKVPLVIPEVNPEAMQNIKAGTGKGALIANPNCSTIRCLKAATPLHRRAKVLRMVVSTYQAASGAGAAAMEELELQTREVLEGKPPTCKIFNRQYAFNLFSHNASVLSNGYNEEEMKMVKETRKIWNDKDVKVTATCIRVPIMRAHAESVNLQFERPLDEDTARDILKNAPGVVVIDDRESNHFPTPLEVSNKDDVAVGRIRQDLSQDGNQGLDIFVCGDQIRKGAALNAIQIAEMLL

SEQ ID NO: 50 - DNA, 1609 bp, Triticum aestivum
caccaccacccacctacccaaatcccagccgccctaaaaccctaggccgccaaacccgccgccgccgccgccgcaatgcaggccgccgcagccgtccaccggccacacctcctcgcggcgtccccgctcgggggccgcgccagccgccggccctccacggtccgcatggcgctccgcgaggacgggccctccgtggccatcgtgggcgccaccggcgcggtggggcaggagttcctccgcgtcatcaccgcccgcgacttcccctaccgcagcctgcgcctcctcgccagcgagcgctccgcgggcaagcgcatcgacttcgagggccgggactacaccgtccaggacctcgcggcgccgggggccttcgacggggtcgacatcgcgctcttcagcgccggcgggagcatcagccgcgcccacgcgcccgccgccgtcgccagcggcgccgtcgtcgtggataacagctccgcctaccggatggaccccgacgtgccgctcgtcatcccggaggttaaccccgaggccatggccgacgtccggctcgggaaaggggctattgtggccaaccccaactgttccaccatcatctgcctcatggctgtcacgccgctgcatcgccacgccaaggtgaaaaggatggttgtcagcacataccaagcagcaagtggtgctggtgctgcagccatggaagaactcaaacttcagactcgagaggtcttggaaggaaagccaccaacctgtaacattttcagtcaacagtatgcttttaatatattttcgcataatgcacctattgttgaaaatggctataatgaggaagagatgaaaatggtgaaggagaccagaaaaatctggaatgacaaggatgtaagagtaactgcaacttgtatacgggttcctacgatgcgcgcgcatgccgaaagcgtgaatctacagtttgaaaagccacttgatgaggacactgccagagaaatcttgagggcagctcctggtgttaccattagtgacgaccgtgctgccaaccgcttccctacaccactggaggtatcggataaagatgacgtatcagttggtaggattcgccaggacttgtcacaagatgataacagagggttggagttatttgtctgtggagaccagatacgtaaaggcgccgcgctgaacgctgtgcagattgctgaaatgctactgaagtgaccgcctttttaccattgtctcatgtgccacgttgctctatccattgatggattgatgtactctagtcactttcaacccagttttggtcgtcgtcttttttgtaatctgtcaacctagcagaagaagtgtaagacgggctttagtcatctgttgcacacaaaagtgcagccacaagtttagaaaaggagggttttcacttgttcggattttgccttaggttggactttgttgcaagtttgtcgtttgtttcttgaaagctggtctgctgtaactttacccccaaagccctcgagataacgaggcgtcctgtggggacctaaaaaaaaaaaaaaaaaaaaaaaaaaaaccccaaaaaaaaaaaaaaaaaaaaaaaaaaaaaaaaaaaaaaaaaaaaaaaaaaaaaaaaaaa

SEQ ID NO: 51 - protein, 374 aa, Triticum aestivum
MQAAAAVHRPHLLAASPLGGRASRRPSTVRMALREDGPSVAIVGATGAVGQEFLRVITARDFPYRSLRLLASERSAGKRIDFEGRDYTVQDLAAPGAFDGVDIALFSAGGSISRAHAPAAVASGAVVVDNSSAYRMDPDVPLVIPEVNPEAMADVRLGKGAIVANPNCSTIICLMAVTPLHRHAKVKRMVVSTYQAASGAGAAAMEELKLQTREVLEGKPPTCNIFSQQYAFNIFSHNAPIVENGYNEEEMKMVKETRKIWNDKDVRVTATCIRVPTMRAHAESVNLQFEKPLDEDTAREILRAAPGVTISDDRAANRFPTPLEVSDKDDVSVGRIRQDLSQDDNRGLELFVCGDQIRKGAALNAVQIAEMLLK

SEQ ID NO: 52 - protein, 340 aa, Aquifex aeolicus
MGYRVAIVGATGEVGRTFLKVLEERNFPVDELVLYASERSEGKVLTFKGKEYTVKALNKENSFKGIDIALFSAGGSTSKEWAPKFAKDGVVVIDNSSAWRMDPDVPLVVPEVNPEDVKDFKKKGIIANPNCSTIQMVVALKPIYDKAGIKRVVVSTYQAVSGAGAKAIEDLKNQTKAWCEGKEMPKAQKFPHQIAFNALPHIDVFFEDGYTKEENKMLYETRKIMHDENIKVSATCVRIPVFYGHSESISMETEKEISPEEAREVLKNAPGVIVIDNPQNNEYPMPIMAEGRDEVFVGRIRKDRVFEPGLSMWVVADNIRKGAATNAVQIAELLVKEGLI

SEQ ID NO: 53 - DNA, 1727 bp, Glycine max
ttgcaacacacattgtcttgtcggcaaaatcttccaccaacaacacacagccatggcaggctcaaacattctttctcactctccttcccttcccaaaacctacagccactccttaaaccaaaacgcgttatcccaaaagcttttttttctgcccctcaaattcaaagccaccacaaaaccacgtgctctcagagcggttctctcgcagaacgctgtcaaaacctcggtggaggacacaaagaacgctcattttcagcactgtttcaccaaatccgaagatgggtatctgtactgtgagggcctcaaggtgcatgacatcatggaatctgttgagagaagacctttctatttgtacagcaagccccagataactaggaatgttgaagcctacaaggatgcattggaagggttgaactccataattggttatgccattaaggccaataataacttgaagattttggaacatttgaggcacttgggttgtggtgctgtgcttgttagtgggaatgagctgaagttggctcttcgagctggctttgatcccacaaggtgtatctttaatgggaatgggaaaatcttggaggatttggtcttggctgctcaggaaggtgtgtttgtcaacattgatagtgagtttgacttggaaaacattgtagaggctgcaaaaagggctgggaagaaggtcaatgttttacttcggattaatcctgatgtggatccacaggttcatccttatgttgccactgggaataagaactctaaatttggcattagaaatgagaagctgcagtgctttttagatgcagtgaaggaacatcctaatgagctcaaacttgtaggggcccactgccatcttggttcaacaattaccaaggttgacattttcagggatgcagccaccattatgatcaactacattgaccaaatccgagatcagggttttgaagttgattacttaaatattggtggaggacttgggatagattattatcattctggtgccatccttcctacacctagagatctcattgacactgtacgagatcttgttatttcacgtggtcttaatctcatcattgaaccaggaagatcactcattgcaaacacgtgttgcttagttaaccgggtgacaggtgttaaaactaatggatctaaaaacttcattgtaattgatggaagtatggctgaacttatccgccctagtctttatgatgcttaccagcatatagagctggtttcccctgccccgtcaaatgctgaaacagaaacttttgatgtggttggccctgtctgtgagtctgcagatttcttaggaaaaggaagagaacttcctactccagccaagggtactggtttggttgttcatgatgctggtgcttattgcatgagcatggcatcaacctacaatctaaagatgcggcctcctgagtattgggttgaagatgatggatcagtgagcaaaataagacatggagagacttttgaagaccacattcggttttttgaggggctttgagctaataatttatcttgtaggaaagaaggctggagaattgttatgtacttggagtttgaatctttcctcgtcaatgaatgcatgactcttgtagttctgtttcttccgttctaattgaatgttgactcccatgacaggaacagagaataaagttgatttcagttaaaaaaaaaaaaaaaaaaaaaaaaaaaaaaaaaaaaaaaaaaaa

SEQ ID NO: 54 - protein, 505 aa, Glycine max
CNTHCLVGKIFHQQHTAMAGSNILSHSPSLPKTYSHSLNQNALSQKLFFLPLKFKATTKPRALRAVLSQNAVKTSVEDTKNAHFQHCFTKSEDGYLYCEGLKVHDIMESVERRPFYLYSKPQITRNVEAYKDALEGLNSIIGYAIKANNNLKILEHLRHLGCGAVLVSGNELKLALRAGFDPTRCIFNGNGKILEDLVLAAQEGVFVNIDSEFDLENIVEAAKRAGKKVNVLLRINPDVDPQVHPYVATGNKNSKFGIRNEKLQCFLDAVKEHPNELKLVGAHCHLGSTITKVDIFRDAATIMINYIDQIRDQGFEVDYLNIGGGLGIDYYHSGAILPTPRDLIDTVRDLVISRGLNLIIEPGRSLIANTCCLVNRVTGVKTNGSKNFIVIDGSMAELIRPSLYDAYQHIELVSPAPSNAETETFDVVGPVCESADFLGKGRELPTPAKGTGLVVHDAGAYCMSMASTYNLKMRPPEYWVEDDGSVSKIRHGETFEDHIRFFEGL

SEQ ID NO: 55 - DNA, 858 bp, Triticum aestivum
tttgagttggagtacctgaatattggaggtggtttggggatagactaccaccacactggtgcagtcttgcctacacctatggatcttatcaacactgtccgggaattggtcctctcacgggatcttactctcattattgaacctggaagatccctgatcgccaatacttgctgcttcgtcaataaggtcactggtgtaaaatcgaatggcacgaagaatttcattgtagttgatggcagcatggccgagctcatcaggcctagtctatatggagcatatcagcatatagaactagtttctccctctccaggtgcagaagtagcaaccttcgatattgttgggccagtctgcgaatctgcagatttccttggcaaagacagggagcttccaacacctgacaagggagctggtttggttgtccacgacgcaggagcctactgcatgagcatggcttcgacctacaacctgaagatgaggccagccgagtattgggtagaggacgatgggtccattgttaagatcaggcacggtgaaacatttgacgactacatgaagttctttgatggtcttcctgcctaggcccttttatcttgttttgggcaagcgtagcccttttcatttgatgagcgcatctcgtggaagattcgtgtgggaaaactattcacttgtttgttatgtgggtcatccccatcaagcatgggggtttttatttgttagaatagagtccaacaagtttagtgattgtagagattgaatggacttactgcattgttatcaattcttgtttatactatataaagggtccgactcctcccaataaagttaaagaatattgttgtttacttttatctaaaaaaa

SEQ ID NO: 56 - protein, 192 aa, Triticum aestivum
FELEYLNIGGGLGIDYHHTGAVLPTPMDLINTVRELVLSRDLTLIIEPGRSLIANTCCFVNKVTGVKSNGTKNFIVVDGSMAELIRPSLYGAYQHIELVSPSPGAEVATFDIVGPVCESADFLGKDRELPTPDKGAGLVVHDAGAYCMSMASTYNLKMRPAEYWVEDDGSIVKIRHGETFDDYMKFFDGLPA

SEQ ID NO: 57 - protein, 526 aa, Arabidopsis thaliana
MGQTNSETQQARLYTQNSQKQLLRSFLLLHLIFGYQSHKTLRMAAATQFLSQPSSLNPHQLKNQTSQRSRSIPVLSLKSTLKPLKRLSVKAAVVSQNSSKTVTKFDHCFKKSSDGFLYCEGTKVEDIMESVERRPFYLYSKPQITRNLEAYKEALEGVSSVIGYAIKANNNLKILEHLRSLGCGAVLVSGNELRLALRAGFDPTKCIFNGNGKSLEDLVLAAQEGVFVNVDSEFDLNNIVEASRISGKQVNVLLRINPDVDPQVHPYVATGNKNSKFGIRNEKLQWFLDQVKAHPKELKLVGAHCHLGSTITKVDIFRDAAVLMIEYIDEIRRQGFEVSYLNIGGGLGIDYYHAGAVLPTPMDLINTVRELVLSRDLNLIIEPGRSLIANTCCFVNHVTGVKTNGTKNFIVIDGSMAELIRPSLYDAYQHIELVSPPPAEAEVTKFDVVGPVCESADFLGKDRELPTPPQGAGLVVHDAGAYCMSMASTYNLKMRPPEYWVEEDGSITKIRHAETFDDHLRFFEGL

SEQ ID NO: 58 - DNA, 1143 bp, Oryza sativa
gcacgaggtcgccgccatcgctgcccttcgcgccctcgatgtcaagtcccacgccgtctccatccacctcaccaagggcctccccctcggctccggcctcggctcctccgccgcctccgccgccgccgctgccaaggccgttgacgccctcttcggctccctcctacaccaagatgacctcgtcctcgcgggcctcgagtccgagaaagccgtcagtggcttccacgccgacaacatcgccccggccatcctcggcggcttcgtcctcgtccgcagctacgaccccttccacctcatcccgctctcctccccacctgccctccgcctccacttcgtcctcgtcacgcccgacttcgaggcgcccaccagcaagatgcgtgccgcgctgcccaaacaggtggccgtccaccagcacgtccgcaactccagccaagcggccgcgcttgtcgccgctgtgctgcaaggggacgccaccctcatcggctccgcaatgtcctccgacggcatcgtggagccaaccagggcgccgctgattcctggcatggctgcggtcaaggccgcggcgttggaagctggggcattgggctgcaccatcagtggagcagggccaactgctgtggctgtcattgacggggaggagaagggcgaggaggttggccggaggatggtggaggcattcgccaatgccggcaatctcaaagcaacagctactgttgctcagctcgatagagttggtgccagggttatctctacctccactttggagtaggaagatctgggaggactgctccggtaggtcaaatttggaatggctcacatggacactagtgggaggagaagaaggggggattggtgtgttttgtaattcctgggctgaccagaacgattgtcagtcagttgggttgtgaattgtgtgatgtagtagcaaactgattcgtgccggcaattgaattgcaataagctagtggttgcagcatcacctggcgaggcgtagctaggagatgcagaaacagcattttgacatgtgtgggtgttgacatgcaacgaataaaatgaatgaagctgaattggggtttaaaaaaaaaaaaaaaaaaaaaaaaaaaaaaaaaaaaaaaaaaaaaaaaaaaaaaaaaaaaaataaaa

SEQ ID NO: 59 - protein, 255 aa, Oryza sativa
HEVAAIAALRALDVKSHAVSIHLTKGLPLGSGLGSSAASAAAAAKAVDALFGSLLHQDDLVLAGLESEKAVSGFHADNIAPAILGGFVLVRSYDPFHLIPLSSPPALRLHFVLVTPDFEAPTSKMRAALPKQVAVHQHVRNSSQAAALVAAVLQGDATLIGSAMSSDGIVEPTRAPLIPGMAAVKAAALEAGALGCTISGAGPTAVAVIDGEEKGEEVGRRMVEAFANAGNLKATATVAQLDRVGARVISTSTLE

SEQ ID NO: 60 - protein, 370 aa, Arabidopsis thaliana
MASLCFQSPSKPISYFQPKSNPSPPLFAKVSVFRCRASVQTLVAVEPEPVFVSVKTFAPATVANLGPGFDFLGCAVDGLGDHVTLRVDPSVRAGEVSISEITGTTTKLSTNPLRNCAGIAAIATMKMLGIRSVGLSLDLHKGLPLGSGLGSSAASAAAAAVAVNEIFGRKLGSDQLVLAGLESEAKVSGYHADNIAPAIMGGFVLIRNYEPLDLKPLKFPSDKDLFFVLVSPEFEAPTKKMRAALPTEIPMVHHVWNSSQAAALVAAVLEGDAVMLGKALSSDKIVEPTRAPLIPGMEAVKKAALEAGAFGCTISGAGPTAVAVIDSEEKGQVIGEKMVEAFWKVGHLKSVASVKKLDKVGARLVNSVSR

SEQ ID NO: 61 - DNA, 1508 bp, Zea mays
aaggatggcgtcgtggtcgtcgccctcagccgccgccaacgccgcctcgggcgcccgattcggccccttcccgagcggagggcagcggctcgcgccgtgtccgtcgctcgtccgcggaactcccgccccgacgctcgtcctcaggctccacccggacggccgtggccatggcctcctcgcgcacaccggcccctctccctcctcgcggtgccgcgccgtcgccgccgaggtcgggggcctcaacatcgccaacgacgtcacccagctcatcggcaacacaccaatggtgtatctcaacaacgtcgtcaagggctctgtcgccaatgtcgctgctaagctcgagattatggagccctgctgtagcgtcaaggacaggatagggtacagtatgataaatgatgctgaacagaagggcttgattactcctggaaagagtgttttggtggaagcaacaagtggaaacacaggcattggtcttgctttcattgctgcttccaaaggatataagctgatactaacaatgccttcatcaatgagcatggagagaagagtcctccttagagcttttggtgccgaacttgtccttactgatgctgcaaaagggatgaaaggggccttagataaggctacagagattttaaacaagacaccaaattcttacatgcttcaacagttcgataaccctgccaaccctcaggtacattatgagactactggtccagagatctgggaggattcaaaggggaaggtggatatattcattggtggaattggaacaggggggacaatatctggtgccggccgttttctcaaggagaaaaatcctggaattaaggttattggtattgagccttctgaaagtaacatactctccggtggaaaacctggtccacataagatccagggaatcggcgcaggatttgttccaaggaacttggatagcgatattcttgatgaagtaattgagatatcaagtgatgaagctgttgagacagcaaaacagttggctgttcaggaaggattactggttggaatctcctctggagcagccgccgctgctgccataaaggttgccaaaagaccagagaatgctggaaagctgatagtggttgtgtttccgagcttcggcgagaggtacctttcatctgtcctctatcagtccataagagaagaatgtgagaacatgcaacctgagccatgagggagccgtcactttaagcgggcatagtaaatgtttctgaaataagacgcgtagccagcatcagtttgctccacttggaatcatttggccatgctcactctatcctttcgctagcctctatgaccggacctaaactggtgtgtgagaaacatccacgactgtcctcccaactgctttcctaaagccaaacgataacactctcaataattgtctatacgattgaagctgatttgattggtaattgtaaacagcttgtctttggatctttgaagtcaaacaaagtcagttggttgaatcaaaaaaaaaa

SEQ ID NO: 62 - protein, 398 aa, Zea mays
MASWSSPSAAANAASGARFGPFPSGGQRLAPCPSLVRGTPAPTLVLRLHPDGRGHGLLAHTGPSPSSRCRAVAAEVGGLNIANDVTQLIGNTPMVYLNNVVKGSVANVAAKLEIMEPCCSVKDRIGYSMINDAEQKGLITPGKSVLVEATSGNTGIGLAFIAASKGYKLILTMPSSMSMERRVLLRAFGAELVLTDAAKGMKGALDKATEILNKTPNSYMLQQFDNPANPQVHYETTGPEIWEDSKGKVDIFIGGIGTGGTISGAGRFLKEKNPGIKVIGIEPSESNILSGGKPGPHKIQGIGAGFVPRNLDSDILDEVIEISSDEAVETAKQLAVQEGLLVGISSGAAAAAAIKVAKRPENAGKLIVVVFPSFGERYLSSVLYQSIREECENMQPEP

SEQ ID NO: 63 - DNA, 1522 bp, Oryza sativa
gcacgaggttctaactacggaactactcccctatccaacacctccgagtccgagcaacgcaagatggcgtcgtggtcgtcgcccgtcgccgccgccgccttgcaggtccatttcgggtcctcctgcttcttctccgcccgatcgccacgacagaccctcctcctaccacctctcgcccgcaaccctacactgaccatccagccccggccccatcccttccggaacatcaactcctcctcctcctccagctggatgtgccacgccgtcgccgccgaggtcgagggcctcaacatcgccgacgacgtcacccagctcatcggcaagactccaatggtatatctcaacaacatcgtcaagggatgtgttgccaatgtcgctgctaagctcgagattatggagccctgttgcagtgtcaaggacaggataggatacagtatgatttctgatgcggaagagaaaggcttgataactcctggaaagagtgttttggtggaaccaacaagtggaaatacaggcattggtcttgccttcattgctgcttccagaggatataaattaatattgaccatgcctgcatcaatgagcatggagagaagagttctactcaaagcttttggcgctgaacttgtccttactgatgccgcaaaagggatgaagggggctgtagataaggctacagagattttaaataagacacctgatgcctatatgctgcagcagtttgacaaccctgccaacccaaaggtacattatgagactactgggccagaaatctgggaggattctaaagggaaggtggatgtattcattggtggaattggaacaggtggaacaatatctggtgctggccgtttcctgaaagagaaaaatcctggaattaaggttattggtattgagccttctgagagtaacatactctctggtggaaaacctggcccacataagattcaaggcattggggcaggatttgttccaaggaacttggatagtgaagttctcgatgaagtgattgagatatctagtgatgaggctgttgagacagcaaagcaattggctcttcaggaaggattactggttggaatttcatctggggcagcagcagcagctgccattaaagttgcaaaaagaccagaaaatgctggaaagttggtagtggttgtgtttccaagctttggtgagaggtacctttcatctatcctttttcagtcgataagagaagaatgtgagaagttgcaacctgaaccatgagcctaacttcagtgttcacaacatcataattgtttctgagatttctggccattagtttttttttctgagaagtatcataccactccatagctgtttgttcgataaataaaacagttacctttgcacttataatgaggcttgtgagggtactgtgaaatttctctgaacatcttctactcttctcttttatccttaaatcaatctgggagcagtttgtaatacatacgtaaatttaaagctgggtgtttggtaattgtaaaaaaaaaaaaaaaaa

SEQ ID NO: 64 - protein, 415 aa, Oryza sativa
ARGSNYGTTPLSNTSESEQRKMASWSSPVAAAALQVHFGSSCFFSARSPRQTLLLPPLARNPTLTIQPRPHPFRNINSSSSSSWMCHAVAAEVEGLNIADDVTQLIGKTPMVYLNNIVKGCVANVAAKLEIMEPCCSVKDRIGYSMISDAEEKGLITPGKSVLVEPTSGNTGIGLAFIAASRGYKLILTMPASMSMERRVLLKAFGAELVLTDAAKGMKGAVDKATEILNKTPDAYMLQQFDNPANPKVHYETTGPEIWEDSKGKVDVFIGGIGTGGTISGAGRFLKEKNPGIKVIGIEPSESNILSGGKPGPHKIQGIGAGFVPRNLDSEVLDEVIEISSDEAVETAKQLALQEGLLVGISSGAAAAAAIKVAKRPENAGKLVVVVFPSFGERYLSSILFQSIREECEKLQPEP

SEQ ID NO: 65 - protein, 383 aa, Spinacia oleracea
MASLVNNAYAAIRTSKLELREVKNLANFRVGPPSSLSCNNFKKVSSSPITCKAVSLSPPSTIEGLNIAEDVSQLIGKTPMVYLNNVSKGSVANIAAKLESMEPCCSVKDRIGYSMIDDAEQKGVITPGKTTLVEPTSGNTGIGLAFIAAARGYKITLTMPASMSMERRVILKAFGAELVLTDPAKGMKGAVEKAEEILKKTPDSYMLQQFDNPANPKIHYETTGPEIWEDTKGKVDIFVAGIGTGGTISGVGRYLKERNPGVQVIGIEPTESNILSGGKPGPHKIQGLGAGFVPSNLDLGVMDEVIEVSSEEAVEMAKQLAMKEGLLVGISSGAAAAAAVRIGKRPENAGKLIAVVFPSFGERYLSSILFQSIREECENMKPE

SEQ ID NO: 66 - protein, 386 aa, Solanum tuberosum
MASFINNPLTSLCNTKSERNNLFKISLYEAQSLGFSKLNGSRKVAFPSVVCKAVSVPTKSSTEIEGLNIAEDVTQLIGNTPMVYLNTIAKGCVANIAAKLEIMEPCCSVKDRIGFSMIVDAEEKGLISPGKTVLVEPTSGNTGIGLAFIAASRGYKLILTMPASMSLERRVILKAFGAELVLTDPAKGMKGAVSKAEEILNNTPDAYILQQFDNPANPKIHYETTGPEIWEDTKGKIDILVAGIGTGGTITGTGRFLKEQNPNIKIIGVEPTESNVLSGGKPGPHKIQGIGAGFIPGNLDQDVMDEVIEISSDEAVETARTLALQEGLLVGISSGAAALAAIQVGKRPENAGKLIGVVFPSYGERYLSSILFQSIREECEKMKPEL

SEQ ID NO: 67 - DNA, 1581 bp, Zea mays
ggccgtggcttactggcttccacccacagccttcgcacttccctccttcctcgcaaatggccgtcgccgtccccaacgctcccggccgcctcttccttctccaatccaccccgttcccgaaccctagcagctcggcatccgccgctcgagcccaatccttccgcgtaccacccctccgcctctcgctattccgacgcatggctgggcgctcgctgacggtgatcgcaggcgcctccggcggctccgaacgagatctcagcgcctccgcagtctccgtggaggccctggactccgtcgcctccgattctgacttagagacgaaggagcccagtgtgtcgacgatgctgacgagcttcgagaactcgttcgacaagtatggggctctgagcacaccgctgtaccagaccgccacctttaagcagccttcagctacagattatggaacttatgattacactagaagtggtaaccctactcgtgatgttctccagagcctcatggctaagcttgagaaagcagatcaagcattctgcttcaccagcgggatggcggcgttagctgcagtaaaacacctccttcaggctggacaagaaatagttgctggtgaggacatatatggtggttctgatcgtctactctcgcaagttgtgccaagaaatggaatagttgtaaaacgagtagatacaacgaaaattagtgatgtggtgtctgcaattggaccctccactagactggtttggctcgaaagtcccacgaaccctcgtcagcaaattactgacattaagacaatctcagagatagcgcattctcatggtgctcttgttttggttgacaacagcatcatgtctccagtgctctcccgtcctatagaactgggagctgatatcgtgatgcactcggctaccaaatttatagcgggacatagtgatcttatggctggaattcttgcagtgaagggtgagagtttggctaaagaggtagggtttctgcaaaatgctgaagggtcgggtctggcaccttttgactgctggctttgcttgaggggaatcaaaaccatggctctgcgggtggagaaacaacaggctaatgcccagaagattgctgaattcctggcgtctcacccgagggtcaagcaagtaaactacgctgggcttcctgaccatcctgggcgagctttacactattcccaggcaaagggagcgggctctgttctcagttttctcaccggctcactggccctctcaaagcacgtcgtggagaccaccaagtacttcagcgtaacagtcagcttcgggagcgtgaagtccctcatcagcctgccgtgcttcatgtcccacgcatcaatccctgcctcggtccgcgaggagcgtggcctaaccgacgacctcgtccggatatcggtcggcatcgaggatgtcgaggacctcatcgccgatctggaccgcgcgctcagaactggcccggtgtagacatcgccgatccttaggtcatgtcaagctatcttttgatgattcattggttgactgcttgcgtgatgataataatgggaatgttgcttggataaaaaaaaaaaaaaaaaaa

SEQ ID NO: 68 - protein, 470 aa, Zea mays
MAVAVPNAPGRLFLLQSTPFPNPSSSASAARAQSFRVPPLRLSLFRRMAGRSLTVIAGASGGSERDLSASAVSVEALDSVASDSDLETKEPSVSTMLTSFENSFDKYGALSTPLYQTATFKQPSATDYGTYDYTRSGNPTRDVLQSLMAKLEKADQAFCFTSGMAALAAVKHLLQAGQEIVAGEDIYGGSDRLLSQVVPRNGIVVKRVDTTKISDVVSAIGPSTRLVWLESPTNPRQQITDIKTISEIAHSHGALVLVDNSIMSPVLSRPIELGADIVMHSATKFIAGHSDLMAGILAVKGESLAKEVGFLQNAEGSGLAPFDCWLCLRGIKTMALRVEKQQANAQKIAEFLASHPRVKQVNYAGLPDHPGRALHYSQAKGAGSVLSFLTGSLALSKHVVETTKYFSVTVSFGSVKSLISLPCFMSHASIPASVREERGLTDDLVRISVGIEDVEDLIADLDRALRTGPV

SEQ ID NO: 69 - DNA, 1685 bp, Oryza sativa
aggcaaccatgagcgccgccgccgccgccgccgccgccgccgcaatccccacctctctcggccgcctcttccacctccgccccaccccgaacccctcccggaaccttagcggcagctcagcgcaacccctcctccgcctcagctaccacccacgcctcacgctctctcgccgcatggaggcgccggcggcgatcgccgactcccacggcggcggcgacctgagcgcgtccgcggtcggcgcggaggcgctgggcgccgtcgccgctccggatttcgatgtggagatgaaggagcctagcgtggcgacgatactgacgagcttcgagaactcgttcgatgggttcgggtctatgagcacgccgctgtaccagacggccacgtttaagcagccttcagcaaccgataatggaccttatgattacactagaagtggtaaccctacacgtgatgttctccaaagccttatggctaagcttgagaaggcggatcaggcattctgcttcaccagtgggatggcagcactagctgcagtaacacacctccttaagtctggacaagaaatagttgctggagaggacatatatggtggctcagaccgtctgctctcacaagttgccccgagacatgggattgtagtaaaacgaattgatacaaccaaaattagtgaggtaacttctgcaattgggcccttgactaaactagtatggcttgaaagtcccaccaatccccgtctacaaattactgatataaagaaaatagcagagatagctcattaccatggtgctcttgttttagtagacaacagcatcatgtctcctgtgctctcccgtcctctagaacttggagcagatattgttatgcactcagcaaccaaatttatagctggacatagcgatcttatggctggaattcttgcggtgaagggtgaaagcagcttggctaaagagattgcatttctacaaaatgctgaaggatcaggtttggcaccatttgattgctggctttgtttgagaggaatcaaaaccatggctttgcgggtggagaagcagcaggctaatgctcagaagattgctgaatttctagcttctcatccaagagtaaagaaagtgaactatgcaggacttcctgatcatcctggacgatctctacactattcccaggcaaagggagcgggttcagttctcagtttcctaactggttcattagctctctcaaaacatgttgttgagaccacaaagtacttcaatgtaacagttagctttggaagtgtgaaatcgctcattagcctgccatgcttcatgtcacacgccagcatcccttctgcggttcgcgaggagcgcggcctgacagacgatctagtcaggatatcggttggaattgaggatgccgacgacctcatagcggatcttgatcatgctctccggtctggtccagcttagagcctgtgaattctgtgcccttcctgttcgttagggatgtagatgtggtcatgtgggtgctatctgtgtgggtgattgattcattggtcaactcaataagctgctgtgtcatcgagggaataaagacaatctatcccaaattttttaacaccatatggtgaccaactgaccatgatatggtcttaatcaattgatatttatagaaggtttctttgaactgcaaaaaaaaaaaaaaaaaaaaaaaaa

SEQ ID NO: 70 - protein, 476 aa, Oryza sativa
MSAAAAAAAAAAIPTSLGRLFHLRPTPNPSRNLSGSSAQPLLRLSYHPRLTLSRRMEAPAAIADSHGGGDLSASAVGAEALGAVAAPDFDVEMKEPSVATILTSFENSFDGFGSMSTPLYQTATFKQPSATDNGPYDYTRSGNPTRDVLQSLMAKLEKADQAFCFTSGMAALAAVTHLLKSGQEIVAGEDIYGGSDRLLSQVAPRHGIVVKRIDTTKISEVTSAIGPLTKLVWLESPTNPRLQITDIKKIAEIAHYHGALVLVDNSIMSPVLSRPLELGADIVMHSATKFIAGHSDLMAGILAVKGESSLAKEIAFLQNAEGSGLAPFDCWLCLRGIKTMALRVEKQQANAQKIAEFLASHPRVKKVNYAGLPDHPGRSLHYSQAKGAGSVLSFLTGSLALSKHVVETTKYFNVTVSFGSVKSLISLPCFMSHASIPSAVREERGLTDDLVRISVGIEDADDLIADLDHALRSGPA

SEQ ID NO: 71 - DNA, 1699 bp, Triticum aestivum
gcacgagagcgtggccacgatactgaccagcttcgagaactcgttcgacaagtatggggctctcagcacgccgctgtaccagacggccaccttcaagcagccttcagcaaccgttaatggagcttatgattatactagaagtggcaaccctactcgtgatgttctccagagccttatggctaagctcgagaaggcagaccaagcattctgcttcactagtgggatggcatcactggctgcagtaacacacctccttcaggctggacaagaaatagttgctggagaggacatatatggtggctctgatcgtctgctctcacaagttgtcccaagaaatggaattgtagtaaaacgggtcgatacaactaaaattaacgacgtgactgctgcaatcggacccttgactagactagtttggcttgaaagtcccaccaatcctcgtcaacaaattactgatataaagaaaatctcagagatagctcattctcatggtgcacttgttttggtggacaacagtatcatgtctccagtgctatcctggcctatagaacttggagcagatattgtgatgcactcagctaccaaatttatagctggacacagtgatcttatggctggaattcttgctgtaaagggtgaaagcttggctaaggagattgcatttctacaaaacgctgaaggttctggtttggcaccttttgattgttggctttgcttgagagggatcaaaaccatggccttacgggtggaaaagcaacaggataatgcccagaagattgctgaattcttagcttctcatccaagggtcaagcaagtgaattatgctggacttcctgatcatcctggccgatctttacactactctcaggcaaagggagcgggctctgtcctcagtttccaaactggttcattgtctctctcaaagcatgttgttgagacaaccaagtacttcaacgtaacagttagcttcggaagtgtgaagtcactcataagcttgccctgcttcatgtcgcacgcgagcatcccttcctcggtgcgagaggagcgtgggttgactgatgatctagtacggatatcggtgggtattgaggatgtggatgacctcatagctgatcttgattacgcgctcaggtccggtccagcatagatcatacaaaatctggactatggcgcttcgggttctagttaatcaagttgtagatgtgatatgcattggtgattcatttgttaagctgcaacagtaataataaacttctgcacgagtattttctgaaatgacgagcccacggttgtatgtgttgttcctcataggcttcaacagaaaaaccctgaggccaactgacaagtagcaacattcataaacttcacaacatcgatacttggttctgcccatgttcatttttcttggctgccattgtgacggctttgtagctcaagtaggaaggagtgacatggccgttggttgatggggagaaaaggagttggttcgtcggatcgatccgtgtaggcgcttgtgtattttgtatatggtgtttttcgtctgtgcaggtgagtctgtgtatacatctggagactggattattcatggtcattggtgtggcggtgaagaataatgtgacgattcttttgtagtgtatctaagaactgtgatgttcttgtgcaaaaaaaaaaaaaaaaaaaaaaaaaaaaaaaaaaaaaa

SEQ ID NO: 72 - protein, 381 aa, Triticum aestivum
HESVATILTSFENSFDKYGALSTPLYQTATFKQPSATVNGAYDYTRSGNPTRDVLQSLMAKLEKADQAFCFTSGMASLAAVTHLLQAGQEIVAGEDIYGGSDRLLSQVVPRNGIVVKRVDTTKINDVTAAIGPLTRLVWLESPTNPRQQITDIKKISEIAHSHGALVLVDNSIMSPVLSWPIELGADIVMHSATKFIAGHSDLMAGILAVKGESLAKEIAFLQNAEGSGLAPFDCWLCLRGIKTMALRVEKQQDNAQKIAEFLASHPRVKQVNYAGLPDHPGRSLHYSQAKGAGSVLSFQTGSLSLSKHVVETTKYFNVTVSFGSVKSLISLPCFMSHASIPSSVREERGLTDDLVRISVGIEDVDDLIADLDYALRSGPA

SEQ ID NO: 73 - DNA, 1267 bp, Glycine max
aaacgaaggcagaaacaggcaaagagctcatagaaattctcgtcctcttcctacataagtcttctcttatcataaacgcgtgttgctcggataaggttgtgctgcggtgattcactcgtactttgcttaatatggcggttgagaagttgagcattgcaaaagatgtgactgaattaattggcaaaactccattggtatatctcaaccgcatcgtggatggctgtgttgccaaagttgctgcgaagctagaaatgatggaaccttgctccagtgtcaaagacaggatagggtatagcatgattgtagatgctgaggagaaaggattcatcacaccgggtgaaagtgtcctcattgagcctactagtggaaacacgggcataggtttggcattcatggcagctgctaagggttataaacttattataaccatgccttcttcgatgagtctcgaaagacgaaccattcttcgggcttttggagctgagttagtcctcactgatcccgccaaaggtatgaaaggagctgttcagaaggcagaagagattcgggataagactccaaattcttatattcttcagcaatttgaaaatcctgccaacccaaagatccattatgaaaccactggaccggagatctggaagggctctagtgggaaggttgatgctcttgtctctgggattgggactggaggtacaataacaggtgctggaaaatatctgaaagatcagaaccctgatataaagctatatggcattgaaccagtagaaagtccaatactgtctggaggaaaacctggccctcataagatccaaggaattggtgctggcttcatccctggtgttctggatgtcgatttacttgatgaagttgttcaaatttcaagtgaagaagccattgaaactgctaaacttcttgctttgaaagaaggtttgctggtgggaatatcatctggtgcagctgcagctgctgcaattaagatagcaaagaggccagaaaatgctggaaaactcattattgcggtcttcccaagctttggagagcgttatttatcatctgtgctctttgaatccgtgaagcgagaagcagagagcatggtcttcgagccttgaatatgaagttatacgagccatttgctgcttttcaagttgcattctctccttgggttgtgtatctattttttctcttatggatctgaaaaataaaaaatggtttatgtttggttgttaattgactacgatttaataaaaaatataagatgtttgctgga

SEQ ID NO: 74 - protein, 325 aa, Glycine max
MAVEKLSIAKDVTELIGKTPLVYLNRIVDGCVAKVAAKLEMMEPCSSVKDRIGYSMIVDAEEKGFITPGESVLIEPTSGNTGIGLAFMAAAKGYKLIITMPSSMSLERRTILRAFGAELVLTDPAKGMKGAVQKAEEIRDKTPNSYILQQFENPANPKIHYETTGPEIWKGSSGKVDALVSGIGTGGTITGAGKYLKDQNPDIKLYGIEPVESPILSGGKPGPHKIQGIGAGFIPGVLDVDLLDEVVQISSEEAIETAKLLALKEGLLVGISSGAAAAAAIKIAKRPENAGKLIIAVFPSFGERYLSSVLFESVKREAESMVFEP

SEQ ID NO: 75 - DNA, 1297 bp, Glycine max
atcagaccatccaaacgaagcagaaagagctcatagaagttctcgtcctcttctctctatcttgaatcttcttctcttatcagaaacgcgcgttgctcggataaggttgtgctgcgccgactcactggtactttgcttaatatggcggctgagaagtcaaacattgcaaaagatgtgactgaattaattggcaaaactccattggtatatctcaaccacatcgtggatggttgtgttgccaaagttgctgccaagcttgaaatgatggaaccttgctccagtgtcaaagacaggataggatatagcatgattgtagacgctgaggagaaaggactcatcacaccgggtgaaagtgtcctcattgaacctaccagtggaaacactggcataggtttggcattcatggcagctgctaagggttataaacttattataaccatgccttcttcgatgagtcttgaaagaagaaccattcttcgtgcttttggagctgagttagttctcactgatcccgccaagggtatgaaaggagctgttcagaaggcagaagagataagggataagactcccaattcttatatgcttcagcaatttgaaaatcctgccaacccaaaggtccattatgaaaccactggaccagagatctggaagggctctagtgggaaggttgatgctcttgtctctgggattgggactggaggtacagtaacaggtgctggaaaatatctgaaagagcagaatcctgatataaagctatatggcattgaaccagtagaaagtccaatactgtctggaggaaaacccggccctcataagatccaaggaattggtgctggcttcatccctggtgttctggatgttgatttacttgatgaagttgttcaaatttcaagtgaagaagctattgaaactgctaagcttcttgcattgaaagaaggtttactggtgggaatatcatctggtgctgctgcagctgctgcagttaagatagcgaagaggccagaaaatgctggaaaactcattgttgcggtcttcccaagttttggagagcgttatctatcgtctgtgctctttgaatccgtgaagcgagaagcagagagcttggtctttgagccttgaatgtcaagtcatacgagccatttgctgcttctcaagttgcattctgtccttgggttgtgtatatattttttctcttatggatctaaaaaataaaaaggatttctgtttgcttgttaattgactaagattcaataaaaatatagtagtttgctgaaaaaaaaaaaaaaaaaaaaaaaaa

SEQ ID NO: 76 - protein, 325 aa, Glycine max
MAAEKSNIAKDVTELIGKTPLVYLNHIVDGCVAKVAAKLEMMEPCSSVKDRIGYSMIVDAEEKGLITPGESVLIEPTSGNTGIGLAFMAAAKGYKLIITMPSSMSLERRTILRAFGAELVLTDPAKGMKGAVQKAEEIRDKTPNSYMLQQFENPANPKVHYETTGPEIWKGSSGKVDALVSGIGTGGTVTGAGKYLKEQNPDIKLYGIEPVESPILSGGKPGPHKIQGIGAGFIPGVLDVDLLDEVVQISSEEAIETAKLLALKEGLLVGISSGAAAAAAVKIAKRPENAGKLIVAVFPSFGERYLSSVLFESVKREAESLVFEP

SEQ ID NO: 77 - DNA, 1567 bp, Glycine max
tttgtgtagtagtgagtgactttgggttgggtttcgtctcaactcaacaaacgtcgtcgttctcccatttccaatctttctccaatggcttcttctgcttccttaatcaactcgttgacttgctcttcgcctgcacccactcagcactgtttcgccttcacccgcaccaccgccaccgctaccgccacgtcactgagacaattcaacagccaccgtccgcccccactcaccacgaggatctcctcgtcgccttccaccgttgtctgcaaagctgtctccgttaagccccaaaccgaaaccgaaggtctcaacattgctgacgatgtcacacagctcatagggaaaacgccgatggtttacctcaacaatatcgtgaagggttctgtggccaatattgctgcgaagcttgagattatggagccctgttgtagtgtcaaggacaggattggttttagtatgataaatgatgctgagcagagaggagctataacacctgggaagagtatattggtggaacctactagtgggaacacaggaattggtcttgcctttatagcagcttcaagaggatataagctaattttgacaatgcctgcttcaatgagtttagaaagacgggttcttttgaaagcatttggggccgagcttgttttgactgatgcagcaaagggcatgaatggagcagtacaaaaagctgaagaaattttaaaaagtacccccaattcatacatgcttcaacaatttgataatccttcaaatcctaaggttcattatgagacaactggcccggagatatgggaagatactagaggaaagatagatattttagttgcaggaattggaaccggtggaactgtttctggagctggccaattcctaaaacaacaaaatccgaaaatacaggttattggcgtagagcctctggaaagcaacatacttacaggtggaaagccaggacctcacaaaattcagggtattggggctggttttgtgcccaggaatttggatcaagatgtgcttgatgaagttatagcgatatcaagtgatgaagcagttgaaactgcaaagcaattagcactccaggaaggcttgctggtgggaatttcttcaggagctgcagctgcagctgctttgaaggttggaaagaggcctgagaatgcaggaaagcttattggggttgtcttcccaagctttggtgaaagatatttgtcaactattcttttccagtcaatacgggaagaatgcgagaaaatgcaacctgagccatgaattgactttgcttgttaatgtttcatactcattttccacaagtatctgtatggaatgacagtgtcagatgtccatgttatgcccgtgtttctcatttttgacactcaagaaattagataactgtgtacaggtcattgatttgacttacaacacagattaatgacccttgattgaagaaattttagatgtaataattattggcattgatgacaacgtagcaaatttctgccttatgttcgctattaaaaaataattgatgaaacagtttatctatttctttttgtacactgccttctct

SEQ ID NO: 78 - protein, 394 aa, Glycine max
MASSASLINSLTCSSPAPTQHCFAFTRTTATATATSLRQFNSHRPPPLTTRISSSPSTVVCKAVSVKPQTETEGLNIADDVTQLIGKTPMVYLNNIVKGSVANIAAKLEIMEPCCSVKDRIGFSMINDAEQRGAITPGKSILVEPTSGNTGIGLAFIAASRGYKLILTMPASMSLERRVLLKAFGAELVLTDAAKGMNGAVQKAEEILKSTPNSYMLQQFDNPSNPKVHYETTGPEIWEDTRGKIDILVAGIGTGGTVSGAGQFLKQQNPKIQVIGVEPLESNILTGGKPGPHKIQGIGAGFVPRNLDQDVLDEVIAISSDEAVETAKQLALQEGLLVGISSGAAAAAALKVGKRPENAGKLIGVVFPSFGERYLSTILFQSIREECEKMQPEP

SEQ ID NO: 79 - DNA, 40 bp, Artificial
  primer described in Example 14
gaattcgcggccgcatggctgttgaaaggtccggaattgc

SEQ ID NO: 80 - DNA, 39 bp, Artificial
  primer described in Example 14
gaattcgcggccgctcagggctcaaaagtcatgctttca

SEQ ID NO: 81 - DNA, 978 bp, Glycine max
atggctgttgaaaggtccggaattgccaaagatgttacggaattgattggtaaaaccccattagtatatctaaataaacttgcggatggttgtgttgcccgggttgctgctaaactggagttgatggagccatgctctagtgtgaaggacaggattgggtatagtatgattgctgatgcagaagagaagggacttatcacacctggaaagagtgtcctcattgagccaacaagtggtaatactggcattggattagccttcatggcagcagccaggggttacaagctcataattacaatgcctgcttctatgagtcttgagagaagaatcattctattagcttttggagctgagttggttctgacagatcctgctaagggaatgaaaggtgctgttcagaaggctgaagagatattggctaagacgcccaatgcctacatacttcaacaatttgaaaaccctgccaatcccaaggttcattatgaaaccactggtccagagatatggaaaggctccgatgggaaaattgatgcatttgtttctgggataggcactggtggtacaataacaggtgctggaaaatatcttaaagagcagaatccgaatataaagctgattggtgtggaaccagttgaaagtccagtgctctcaggaggaaagcctggtccacacaagattcaagggattggtgctggttttatccctggtgtcttggaagtcaatcttcttgatgaagttgttcaaatatcaagtgatgaagcaatagaaactgcaaagcttcttgcgcttaaagaaggcctatttgtgggaatatcttccggagctgcagctgctgctgcttttcagattgcaaaaagaccagaaaatgccgggaagcttattgttgccgtttttcccagcttcggggagaggtacctgtcctccgtgctatttgagtcagtgagacgcgaagctgaaagcatgacttttgagccctga

SEQ ID NO: 82 - DNA, 35 bp, Artificial
  primer described in Example 15
gaattcggatccgatccgtcaacatggtggagcac

SEQ ID NO: 83 - DNA, 35 bp, Artificial
  primer described in Example 15
gaattcgcggccgctgtaattgtaaatagtaattg

SEQ ID NO: 84 - DNA, 35 bp, Artificial
  primer described in Example 16
gaattcggatccactacgctttgaaggagcacgtg

SEQ ID NO: 85 - DNA, 35 bp, Artificial
  primer described in Example 16
gaattcgcggccgccgtgaaacctcagtgcaagaa

SEQ ID NO: 86 - DNA, 40 bp, Artificial
  primer described in Example 17
gaattcgcggccgcgcggttgagaagttgagcattgcaaa

SEQ ID NO: 87 - DNA, 39 bp, Artificial
  primer described in Example 17
gaattcgcggccgctgccaaacctatgcccgtgtttcca

SEQ ID NO: 88 - DNA, 978 bp, Glycine max
atggcggttgagaagttgagcattgcaaaagatgtgactgaattaattggcaaaactccattggtatatctcaaccgcatcgtggatggctgtgttgccaaagttgctgcgaagctagaaatgatggaaccttgctccagtgtcaaagacaggatagggtatagcatgattgtagatgctgaggagaaaggattcatcacaccgggtgaaagtgtcctcattgagcctactagtggaaacacgggcataggtttggcattcatggcagctgctaagggttataaacttattataaccatgccttcttcgatgagtctcgaaagacgaaccattcttcgggcttttggagctgagttagtcctcactgatcccgccaaaggtatgaaaggagctgttcagaaggcagaagagattcgggataagactccaaattcttatattcttcagcaatttgaaaatcctgccaacccaaagatccattatgaaaccactggaccggagatctggaagggctctagtgggaaggttgatgctcttgtctctgggattgggactggaggtacaataacaggtgctggaaaatatctgaaagatcagaaccctgatataaagctatatggcattgaaccagtagaaagtccaatactgtctggaggaaaacctggccctcataagatccaaggaattggtgctggcttcatccctggtgttctggatgtcgatttacttgatgaagttgttcaaatttcaagtgaagaagccattgaaactgctaaacttcttgctttgaaagaaggtttgctggtgggaatatcatctggtgcagctgcagctgctgcaattaagatagcaaagaggccagaaaatgctggaaaactcattattgcggtcttcccaagctttggagagcgttatttatcatctgtgctctttgaatccgtgaagcgagaagcagagagcatggtcttcgagccttga

SEQ ID NO: 89 - protein, 292 aa, Populus alba  X Populus tremula
RIAAKLEMMEPCSSVKDRIGYSMITDAEEKGLIKAGESVLIEPTSGNTGIGLAFMAAAKGYRLIITMPASMSLERRMVLLAFGAELVLTDPARGMKGAVQKAEEISAKTPNSYILQQFENPANPKVHYDTTGPEIWKGSGGKVDAFVSGIGTGGTITGAGKYLKEQNPDIKLYGVEPVESAVLSGGKPGPHKIQGIGAGFIPGVLNVDLLDETVQISSEEAIETAKLLALKEGLLVGISSGAAAAAAIKIAKRPENAGKLIVAIFPSFGERYLSSVLFESVKKEAENMVFEP

SEQ ID NO: 90 - protein, 386 aa, Solanum tuberosum
MASFINNPLTSLCNTKSEANNLFKISPLRAQSLGFSKLNGSRKVAFPSVVCKAVSVPTKSSTEIEGLNIAEDVTQLIGNTPMVYLNTIAKGCVANIAAKLEIMEPCCSVKDRIGFSMIVDAEEKGLISPGKTVLVEPTSGNTGIGLAFIAASRGYKLILTMPASMSLERRVILKAFGAELVLTDPAKGMKGAVSKAEEILNNTPDAYILQQFDNPANPKIHYETTGPEIWEDTKGKIDILVAGIGTGGTITGTGRFLKEQNPNIKIIGVEPTESNVLSGGKPGPHKIQGIGAGFIPGNLDQDVMDEVIEISSDEAVETAKQLALQEGLLVGISSGAAALAAIQVGKRPENAGKLIGVVFPSYGERYLSSILFQSIREECEKMKPEL

SEQ ID NO: 91 - protein, 325 aa, Glycine max
  ref <302>: TITLE: New Recombinant DNA Construct
  ref <308>: DATABASE ACCESSION NUMBER: USPATALL / SEQ ID NO:198006
  ref <309>: DATABASE ENTRY DATE: 2004-09-24
  ref <310>: PATENT DOCUMENT NUMBER: US2004031072
  ref <311>: PATENT FILING DATE: 1999-05-06
  ref <312>: PUBLICATION DATE: 2004-02-12
  ref <313>: RELEVANT RESIDUES: (1)..(325)
MAVEKLSIAKDVTELIGKTPLVYLNRIVDGCVAKVAAKLEMMEPCSSVKDRIGYSMIVDAEEKGLITPGESVLIEPTSGNTGIGLAFMAAAKGYKLIITMPSSMSLERRTILRAFGAELVLTDPAKGMKGAVQKAEEIRDKTPNSYILQQFENPANPKIHYETTGPEIWKGSSGKVDALVSGIGTGGTITGAGKYLKDQNPDIKLYGIEPVESPILSGGKPGPHKIQGIGAGFIPGVLDVDLLDEVVQISSEEAIETAKLLALKEGLLVGISSGAAAAAAIKIAKRPENAGKLIIAVFPSFGERYLSSVLFESVKREAESMVFEP

SEQ ID NO: 92 - protein, 394 aa, Glycine max
  ref <302>: TITLE: New Recombinant DNA Construct
  ref <308>: DATABASE ACCESSION NUMBER: USPATALL / SEQ ID NO:129998
  ref <309>: DATABASE ENTRY DATE: 2004-10-07
  ref <310>: PATENT DOCUMENT NUMBER: US2004123343
  ref <311>: PATENT FILING DATE: 2000-04-19
  ref <312>: PUBLICATION DATE: 2004-06-24
  ref <313>: RELEVANT RESIDUES: (1)..(394)
MASWSSPVAAAALQVHFGSSCFFSARSPRQTLLLPPLARNPTLTIQPRPHPFRNINSSSSSSWMCHAVAAEVEGLNIADDVTQLIGKTPMVYLNNIVKGCVANVAAKLEIMEPCCSVKDRIGYSMISDAEEKGLITPGKSVLVEPTSGNTGIGLAFIAASRGYKLILTMPASMSMERRVLLKAFGAELVLTDAAKGMKGAVDKATEILNKTPDAYMLQQFDNPANPKVHYETTGPEIWEDSKGKVDVFIGGIGTGGTISGAGRFLKEKNPGIKVIGIEPSESNILSGGKPGPHKIQGIGAGFVPRNLDSEVLDEVIEISSDEAVETAKQLALQEGLLVGISSGAAAAAAIKVAKRPENAGKLVVVVFPSFGERYLSSILFQSIREECEKLQPEP

SEQ ID NO: 93 - protein, 286 aa, Glycine max
MPTGLPAANSLVAPDEEGWVWGQIKAEARRDAESEPALASYLYSTILSHSSLERSLSFHLGNKLCSSTLLSTLLYDLFLNAFSSDPSLRSAAVADLRAARERDPACVSYSHCLLNYKGFLACQAHRVAHLLWRQSRRPLALALHSRIADVFAVDIHPPARIGKGILFDHATGVVVRETASIGNNVSILHHVTLGGTGKVGGDRHPKIGDGVLIGAGATILGNIKIGEGAKVGAGSVVLIDVPPRTTAVGNPARLVGGKEKPSKHEDVPGESMDHTSFISEWSDYII

SEQ ID NO: 94 - protein, 157 aa, Glycine max
ARAHKLWLQGRKVLALLIQNRVSEVFAVDIHPGAKIGRGILLDHATGLVVGETAVIGNNVSILHNVTLGGTGKASGDRHPKIGDGVLIGAGTCILGNIKIGDGAKIGACSVVLKEVPPRTTAVGNPARLVGGKDNPIKLDKMPSFTMDHTSWSDYVI

(19) United States
(12) Patent Application Publication
(10) Pub. No.: US 2006/0026705 A1
(43) Pub. Date: Feb. 2, 2006

(54) Plant amino acid biosynthetic enzymes

(76) Inventors: Stephen M. Allen, Wilmington, DE (US); Anthony J. Kinney, Wilmington, DE (US); Zhan-Bin Liu, (US); Kevin L. Stecca, Bear, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/057,012

(22) Filed: Feb. 11, 2005

Related U.S. Application Data
(63) Continuation-in-part of application No. 09/931,457, filed on Feb. 22, 2002.
Application No. 09/931,457 is a continuation-in-part of application No. 09/424,976, filed on Dec. 2, 1999, now abandoned.
(60) Provisional application No. 60/049,406, filed on Jun. 12, 1997.
(60) Provisional application No. 60/065,385, filed on Nov. 12, 1997.

(51) Int. Cl. A01H 1/00; C07H 21/04; C12N 9/10; C12N 5/04; C12N 15/82

(52) U.S. Cl. 800/278; 435/468; 435/419; 435/193; 536/23.2

(57) ABSTRACT

An isolated polynucleotide encoding a plant cysteine synthase is disclosed. Also disclosed is the construction of a recombinant construct comprising all or part of a coding or non-coding region of the isolated polynucleotide for use in co-suppression or antisense suppression of endogenous nculeic acid sequences encoding polypeptides having cysteine synthase activity. In another aspect, use of this construct to alter levels of cysteine synthase in a plant is disclosed.

This application is a continuation-in-part of application Ser. No. 09/931,457 filed on Aug. 16, 2001, pending, which is a continuation-in-part of application Ser. No. 09/424,976 filed on Dec. 2, 1999, abandoned, which is a national stage application of PCT/US98/12073 with an International filing date of Jun. 11, 1998, which in turn claims priority benefit of U.S. Provisional Application No. 60/049,406, filed Jun. 12, 1997, and U.S. Provisional Application No. 60/065,385, filed Nov. 12, 1997.

### FIELD OF THE INVENTION

This invention is in the field of plant molecular biology. More specifically, this invention pertains to nucleic acid fragments encoding enzymes involved in amino acid biosynthesis in plants and seeds.

### BACKGROUND OF THE INVENTION

Many vertebrates, including humans, lack the ability to manufacture a number of amino acids and therefore require these amino acids in their diet. These are called essential amino acids. Grain-derived foods or feed, however, are deficient in certain essential amino acids, such as lysine, the sulfur-containing amino acids methionine and cysteine, threonine and tryptophan. For example, in corn (Zea mays L.) lysine is the most limiting amino acid for the dietary requirements of many animals, and soybean (Glycine max L.) meal is used as an additive to corn-based animal feeds primarily as a lysine supplement. Often microbial-fermentation produced lysine is needed for such supplementation. Thus, an increase in lysine content of either corn or soybean would reduce or eliminate the need to supplement mixed grain feeds with lysine produced via fermentation.

Furthermore, in corn the sulfur amino acids are the third most limiting amino acids, after lysine and tryptophan, for the dietary requirements of many animals. Legume plants, however, while rich in lysine and tryptophan, have low sulfur-containing amino acid content. Therefore, the use of soybean meal to supplement corn in animal feed is not satisfactory. An increase in the sulfur amino acid content of either corn or soybean would improve the nutritional quality of the mixtures and reduce the need for further supplementation through addition of more expensive methionine.

One approach to increasing the nutritional quality of human foods and animal feed is to increase the production and accumulation of specific free amino acids via genetic engineering of the biosynthetic pathway of the essential amino acids. Biosynthetically, lysine, threonine, methionine, cysteine and isoleucine are all derived from aspartate. Regulation of the biosynthesis of each member of this family is interconnected (see FIG. 1). The organization of the pathway leading to biosynthesis of lysine, threonine, methionine, cysteine and isoleucine indicates that over-expression or reduction of expression of genes encoding, inter alia, aspartic semialdehyde dehydrogenase, homoserine kinase, diaminopimelate decarboxylase, cysteine synthase and cystathionine β-lyase in corn and soybean could be used to alter levels of these amino acids in human food and animal feed. However, few of the genes encoding enzymes that regulate this pathway in plants, especially corn and soybeans, are available. Accordingly, availability of nucleic acid sequences encoding all or a portion of these enzymes would facilitate development of nutritionally improved crop plants.

### SUMMARY OF THE INVENTION

In a first embodiment, this invention relates to an isolated polynucleotide sequence comprising:
(a) a nucleotide sequence encoding a polypeptide having cysteine synthase activity, wherein the polypeptide has an amino acid sequence of at least 90% sequence identity, based on the Clustal method of alignment, when compared to one of SEQ ID NO:74, 76 or 78;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

In a second embodiment, this invention concerns a recombinant construct comprising this isolated polynucleotide operably linked to at least one regulatory sequence. Also of interest, are cells and plants comprising this recombinant construct and seeds obtained from such plants.

In a third embodiment, this invention concerns a method of altering the level of sulfur-containing amino acids in a soybean plant which comprises:
(a) transforming a soybean plant cell with a recombinant DNA construct of the inventioin
(b) regenerating a soybean plant from the transformed plant cell of step (a); and
(c) comparing the sulfur-containing amino acid levels of a transformed soybean plant with the sulfur-containing amino acid levels of an untransformed soybean plant.

In a fourth embodiment, this invention concerns an isolated polynucleotide sequence comprising:
(a) a nucleotide sequence encoding a polypeptide having cysteine synthase activity, wherein the polypeptide has an amino acid sequence of greater than 87.1% sequence identity, based on the Clustal method of alignment, when compared to SEQ ID NO:31;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

In a fifth embodiment, this invention concerns a recombinant construct comprising this isolated polynucleotide operably linked to at least one regulatory sequence. Also of interest, are cells and plants comprising this recombinant construct and seeds obtained from such plants.

In a sixth embodiment, this invention concerns a method of altering the level of sulfur-containing amino acids in a soybean plant which comprises:
(a) transforming a soybean plant cell with a recombinant DNA construct of the invention;
(b) regenerating a soybean plant from the transformed plant cell of step (a); and
(c) comparing the sulfur-containing amino acid levels of a transformed soybean plant with the sulfur-containing amino acid levels of an untransformed soybean plant.

In a seventh embodiment, this invention concerns a method for producing a soybean plant having an altered level of sulfur-containing amino acids which comprises:
(a) crossing an agronomically elite soybean line with a plant transformed with one of the recombinant constructs of the invention, and
(b) screening the seed of progeny plants obtained from step (a) for an altered level of sulfur-containing amino acids compared to an untransformed soybean plant.

In an eighth embodiment, this invention concerns a method for producing a soybean protein product having an altered level of sulfur-containing amino acids which comprises:
(a) crossing an agronomically elite soybean line with a plant transformed with one of the recombinant constructs of the invention; and
(b) screening the seed of progeny plants obtained from step (a) for an altered level of sulfur-containing amino acids compared to an untransformed soybean plant; and
(c) processing the seed selected in step (b) to obtain the desired soybean protein product.

In a ninth embodiment, this invention concerns a soybean protein product made from the seed obtained from transformed plant of the invention.

In a tenth embodiment, this invention concerns food or feed which incorporates such soybean protein product.

In an eleventh embodiment, this invention concerns a method of increasing the level of sulfur-containing amino acids in a soybean plant which comprises:
(a) transforming a soybean plant cell with a recombinant DNA construct comprising an isolated polynucleotide sequence comprising:
   (i) a nucleotide sequence encoding a polypeptide having cysteine synthase activity, wherein the polypeptide has an amino acid sequence of at least 90% sequence identity, based on the Clustal method of alignment, when compared to one of SEQ ID NO:74;
   (ii) the full-length complement of the nucleotide sequence of (i); or
   (iii) all or part of a coding or non-coding region of the isolated polynucleotide sequence comprising any of the nucleotide sequences of (i) or (ii) for use in root specific co-suppression or antisense suppression of endogenous nucleic acid sequences encoding polypeptides having cysteine synthase activity wherein said isolated polynucleotide is operably linked to at least one regulatory sequence, said regulatory sequence being a root specific promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

In a twelfth embodiment, this invention concerns a method of increasing the level of sulfur-containing amino acids in a soybean plant which comprises:
(a) transforming a soybean plant cell with a recombinant DNA construct comprising an isolated polynucleotide sequence comprising:
   (i) a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31;
   for use in overexpressing endogenous nucleic acid sequences encoding polypeptides having cysteine synthase activity in the transformed plant
   wherein said isolated polynucleotide is operably linked to at least one regulatory sequence, said regulatory sequence being a constitutive promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

In a thirteenth embodiment, this invention concerns a method of increasing the level of sulfur-containing amino acids in a soybean plant which comprises:
(a) transforming a soybean plant cell with a recombinant DNA construct comprising an isolated polynucleotide sequence comprising a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31; for use in seed-specific overexpression of endogenous nucleic acid sequences encoding polypeptides having cysteine synthase activity
wherein said isolated polynucleotide is operably linked to at least one regulatory sequence, said regulatory sequence being a seed-specific promoter.
(b) regenerating a soybean plant from the transformed plant cell of step (a); and
(c) comparing the sulfur-containing amino acid levels of seeds obtained from a transformed soybean plant with the sulfur-containing amino acid levels of seeds obtained from an untransformed soybean plant.

### BRIEF DESCRIPTION OF THE DRAWINGS AND SEQUENCE LISTINGS

The invention can be more fully understood from the following detailed description and the accompanying drawings and Sequence Listing which form a part of this application.

FIG. 1 depicts the biosynthetic pathway for the aspartate family of amino acids. The following abbreviations are used: AK=aspartokinase; ASADH=aspartic semialdehyde dehydrogenase; DHDPS=dihydrodipicolinate synthase; DHDPR=dihydrodipicolinate reductase; DAPEP=diaminopimelate epimerase; DAPDC=diaminopimelate decarboxylase; HDH=homoserine dehydrogenase; HK=homoserine kinase; TS=threonine synthase; TD=threonine deaminase; CγS=cystathionine γ-synthase; CβL=cystathionine β-lyase; MS=methionine synthase; CS=cysteine synthase; and SAMS=S-adenosylmethionine synthase.

FIGS. 2 through 6 show the amino acid sequence alignments between the known art sequences for aspartic semialdehyde dehydrogenase, diaminopimelate decarboxylase, homoserine kinase, cysteine synthase, and cystathione β-lyase with the sequences included in this application. Alignments were performed using the Clustal alogarithm described in Higgins and Sharp (1989) (CABIOS 5:151-153). Amino acids conserved among all sequences are indicated by an asterisk (*) above the alignment. Dashes are used by the program to maximize the alignment. A description of FIGS. 2 through 6 follows:

FIGS. 2A, 2B, 2C and 2D show a comparison of the aspartic semialdehyde dehydrogenase amino acid sequences from corn contig assembled from clones p0003.cgpha22r:fis, cpe1c.pk009.b24, p0016.ctscp83r, and p0075.cslab16r (SEQ ID NO:43), rice clone rlr48.pk0003.d12 (SEQ ID NO:2), the contig of 5′ RACE PCR and rice clone rlr48.pk0003.d12 (SEQ ID NO:45), soybean clones sfl1.pk0122.f9 (SEQ ID NO:6), ses9c.pk001.a15:fis (SEQ ID NO:47), and sfl1.pk0122.f9:fis (SEQ ID NO;49), wheat clones wr1.pk0004.c11 (SEQ ID NO:4) and wdk1c.pk014.n5:fis (SEQ ID NO:51) with the Legionella pneumophila (NCBI General Identifier No. 2645882; SEQ ID NO:7) and the Aquifex aeolicus sequences (NCBI General Identifier No. 6225258; SEQ ID NO:52). FIG. 2A: positions 1 through 120; FIG. 2B: positions 121 through 240; FIG. 2C: positions 241 through 360; FIG. 2D: positions 361 through 392.

FIGS. 3A, 3B, 3C, 3D and 3E show a comparison of the diaminopimelate decarboxylase amino acid sequences derived from corn clones cen3n.pk0067.a3 (SEQ ID NO:9) and cr1 n.pk0103.d8 (SEQ ID NO:11), rice clone rl0n.pk0013.b9 (SEQ ID NO:13), soybean clones sr1.pk0132.c1 (SEQ ID NO:15), sdp3c.pk001.o15 (SEQ ID NO:19) and sdp3c.pk001.o15:fis (SEQ ID NO:54), wheat clones wlk1.pk0012.c2 (SEQ ID NO:17) and wlk1.pk0012.c2:fis (SEQ ID NO:56) with the Pseudomonas aeruginosa (NCBI General Identifier No. 118304; SEQ ID NO:20) and Arabidopsis thaliana sequences (NCBI General Identifier No. 9279586; SEQ ID NO:57). FIG. 3A: positions 1 through 120; FIG. 3B: positions 121 through 240; FIG. 3C: positions 241 through 360; FIG. 3D: positions 361 through 480; FIG. 3E: positions 481 through 535.

FIGS. 4A, 4B and 4C show a comparison of the homoserine kinase amino acid sequences derived from corn clone cr1 n.pk0009.g4 (SEQ ID NO:22), rice clones rca1c.pk005.k3 (SEQ ID NO:24) and rca1c.pk005.k3:fis (SEQ ID NO:59), soybean clone ses8w.pk0020.b5 (SEQ ID NO:26), wheat clone wl1n.pk0065.f2 (SEQ ID NO:28) with the Methanococcus jannaschii (NCBI General Identifier No. 1591748; SEQ ID NO:29) and the Arabidopsis thaliana sequences (NCBI General Identifier No. 4927412; SEQ ID NO:60). FIG. 4A: positions 1 through 180; FIG. 4B: positions 181 though 360; FIG. 4C: positions 361 through 396.

FIGS. 5A, 5B and 5C show a comparison of the cysteine synthase amino acid sequences derived from the corn contig assembled from clones cco1n.pk083.j4, chp2.pk0016.b1, cpd1c.pk004.b20, cr1n.pk0083.c5, csi1.pk0003.g6, and p0126.cnlcb49r (SEQ ID NO:62), rice clone rls6.pk0068.b7:fis (SEQ ID NO:64), soybean clone se3.05h06 (SEQ ID NO:31) with the Citrullus lanatus sequence (NCBI General Identifier No. 540497; SEQ ID NO:32), the Spinacia oleracea sequence (NCBI General Identifier No. 416869; SEQ ID NO:65), and the Solanum tuberosum sequence (NCBI General Identifier No. 11131628; SEQ ID NO:66). FIG. 5A: positions 1 through 180; FIG. 5B: positions 181 through 360; FIG. 5C: positions 361 through 424.

FIGS. 6A, 6B, 6C and 6D show a comparison of the amino acid sequences of the cystathionine β-lyase derived from corn clone cen1.pk0061.d4 (SEQ ID NO:34), corn contig assembled from clones p0005.cbmei71r, p0014.ctuui39r, p0109.cdadg47r, and p0125.czaay16r (SEQ ID NO:68), rice clone rlr12.pk0026.g1 (SEQ ID NO:36), the contig of 5′ PCR and rice clone rlr12.pk0026.g1:fis (SEQ ID NO:70), soybean clone sfl1.pk0012.c4 (SEQ ID NO:38), and wheat clones wr1.pk0091.g6 (SEQ ID NO:40) and wr1.pk0091.g6:fis (SEQ ID NO:72) with the Arabidopsis thaliana sequence (NCBI General Identifier No. 1708993; SEQ ID NO:41). FIG. 6A: positions 1 through 120; FIG. 6B: positions 121 through 240; FIG. 6C: positions 241 through 360; FIG. 6D: positions 361 through 483.

FIGS. 7A, 7B and 7C present an alignment of the amino acid sequences set forth in SEQ ID NOs:31, 74, 76 and 78, the amino acid sequence from Citrullus lanatus (NCBI General Identifier No. 540497; SEQ ID NO:32), O-acetylserine (thiol)lyase from Populus alba x Populus tremula (NCBI General Identifier No. 34099833; SEQ ID NO:89), plastidic cysteine synthase 1 from Solanum tuberosum (NCBI General Identifier No. 12081919; SEQ ID NO:90), SEQ ID NO:198006 in U.S. 20004031072 (SEQ ID NO:91) and SEQ ID NO:129998 in U.S. 20004123343 (SEQ ID NO:92).

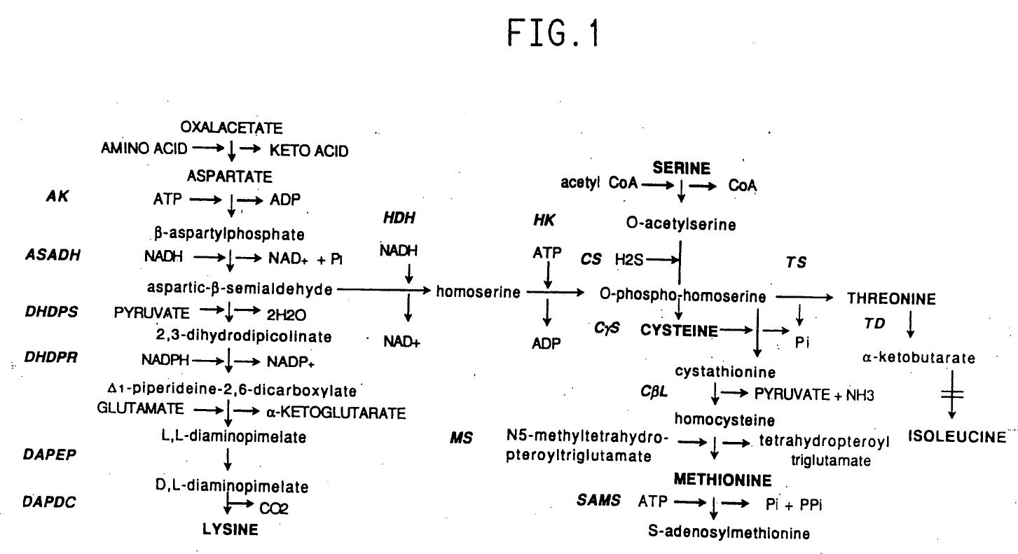

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. The sequence descriptions and Sequence Listing attached hereto comply with the rules governing nucleotide and/or amino acid sequence disclosures in patent applications as set forth in 37 C.F.R. §1.821-1.825.

TABLE 1
Plant Biosynthetic Enzymes

| | | SEQ ID NO: | |
|---|---|---|---|
| Polypeptide | Clone | (Nucleotide) | (Amino Acid) |
| rice ASADH | rlr48.pk0003.d12 | 1 | 2 |
| wheat ASADH | wr1.pk0004.c11 | 3 | 4 |
| soybean ASADH | sfl1.pk0122.f9 | 5 | 6 |
| L. pneumophila | NCBI GI 2645882 | | 7 |
| ASADH | | | |
| corn DAPEP | cen3n.pk0067.a3 | 8 | 9 |
| corn DAPEP | cr1n.pk0103.d8 | 10 | 11 |
| rice DAPEP | rl0n.pk0013.b9 | 12 | 13 |
| soybean DAPEP | sr1.pk0132.c1 | 14 | 15 |
| wheat DAPEP | wlk1.pk0012.c2 | 16 | 17 |
| soybean DAPEP | sdp3c.pk001.o15 | 18 | 19 |
| P. aeruginosa | NCBI GI 118304 | | 20 |
| DAPEP | | | |
| corn HK | cr1n.pk0009.g4 | 21 | 22 |
| rice HK | rca1c.pk005.k3 | 23 | 24 |
| soybean HK | ses8w.pk0020.b5 | 25 | 26 |
| wheat HK | wl1n.pk0065.f2 | 27 | 28 |
| M. jannaschii HK | NCBI GI 1591748 | | 29 |
| soybean CS | se3.05h06 | 30 | 31 |
| C. lanatus CS | NCBI GI 540497 | | 32 |
| corn CβL | cen1.pk0061.d4 | 33 | 34 |
| rice CβL | rlr12.pk0026.g1 | 35 | 36 |
| soybean CβL | sfl1.pk0012.c4 | 37 | 38 |
| wheat CβL | wr1.pk0091.g6 | 39 | 40 |
| A. thaliana CβL | NCBI GI 1708993 | | 41 |
| corn ASADH | Contig of: | 42 | 43 |
| | p0003.cgpha22r:fis | | |
| | cpe1c.pk009.b24 | | |
| | p0016.ctscp83r | | |
| | p0075.cslab16r | | |
| rice ASADH | 5′ RACE PCR+ | 44 | 45 |
| | rlr48.pk0003.d12 | | |
| soybean ASADH | ses9c.pk001.a15:fis | 46 | 47 |
| soybean ASADH | sfl1.pk0122.f9:fis | 48 | 49 |
| wheat ASADH | wdk1c.pk014.n5:fis | 50 | 51 |
| A. aeolicus ASADH | NCBI GI 6225258 | | 52 |
| soybean DAPEP | sdp3c.pk001.o15:fis | 53 | 54 |
| wheat DAPEP | wlk1.pk0012.c2:fis | 55 | 56 |
| A. thaliana DAPEP | NCBI GI 9279586 | | 57 |
| rice HK | rca1c.pk005.k3:fis | 58 | 59 |
| A. thaliana HK | NCBI GI 4927412 | | 60 |
| corn CS | Contig of: | 61 | 62 |
| | cco1n.pk083.j4 | | |
| | chp2.pk0016.b1 | | |
| | cpd1c.pk004.b20 | | |
| | cr1n.pk0083.c5 | | |
| | csi1.pk0003.g6 | | |
| | p0126.cnlcb49r | | |
| rice CS | rls6.pk0068.b7:fis | 63 | 64 |
| S. oleracea CS | NCBI GI 416869 | | 65 |
| S. tuberosum CS | NCBI GI 11131628 | | 66 |
| corn CβL | Contig of: | 67 | 68 |
| | p0005.cbmei71r | | |
| | p0014.ctuui39r | | |
| | p0109.cdadg47r | | |
| | p0125.czaay16r | | |
| rice CβL | 5′RACE PCR + | 69 | 70 |
| | rlr12.pk0026.g1:fis | | |
| wheat CβL | wr1.pk0091.g6:fis | 71 | 72 |

The nucleotide and amino acid sequences shown in SEQ ID NOs:1 through 41 are found, with the same SEQ ID NO, in U.S. application Ser. No. 09/424,976. All or a portion of some of the sequences in the present application are found in the provisional applications for which the present application claims priority to. Table 1A indicates the SEQ ID NO: in application Ser. No. 09/931,457 filed on Aug. 16, 2001, and the corresponding SEQ ID NO: in the previously-filed provisional applications.

TABLE 1A
Sequence Priority

| Application | Provisional Application | Provisional Application |
|---|---|---|
| No. 09/424, 976 | No. 60/049406 | No. 60/065385 |
| SEQ ID NO: 1 | SEQ ID NO: 1 | |
| SEQ ID NO: 2 | SEQ ID NO: 2 | |
| SEQ ID NO: 3 | SEQ ID NO: 3* | |
| SEQ ID NO: 4 | SEQ ID NO: 4* | |
| SEQ ID NO: 8 | SEQ ID NO: 7 | SEQ ID NO: 8 |
| SEQ ID NO: 9 | SEQ ID NO: 8 | SEQ ID NO: 9 |
| SEQ ID NO: 12 | SEQ ID NO: 9 | |
| SEQ ID NO: 13 | SEQ ID NO: 10 | |
| SEQ ID NO: 14 | SEQ ID NO: 11 | SEQ ID NO: 5 |
| SEQ ID NO: 15 | SEQ ID NO: 12 | SEQ ID NO: 6 |
| SEQ ID NO: 21 | SEQ ID NO: 13 | SEQ ID NO: 10* |
| SEQ ID NO: 22 | SEQ ID NO: 14 | SEQ ID NOs: 11* and 14* |
| SEQ ID NO: 23 | SEQ ID NO: 17* | SEQ ID NO: 15 |
| SEQ ID NO: 24 | SEQ ID NO: 18* | SEQ ID NO: 16 |
| SEQ ID NO: 25 | SEQ ID NO: 15 | SEQ ID NO: 13 |
| SEQ ID NO: 26 | SEQ ID NO: 16 | SEQ ID NO: 14 |
| SEQ ID NO: 30 | SEQ ID NO: 19 | SEQ ID NO: 17 |
| SEQ ID NO: 31 | SEQ ID NO: 20 | SEQ ID NO: 18 |
| SEQ ID NO: 33* | SEQ ID NO: 21 | SEQ ID NO: 19 |
| SEQ ID NO: 34 | SEQ ID NO: 22 | SEQ ID NO: 20 |
| SEQ ID NO: 37 | SEQ ID NO: 23 | SEQ ID NO: 21* |
| SEQ ID NO: 38 | SEQ ID NO: 24 | SEQ ID NO: 22* |

*Indicates that only a portion of the sequence was in the application.

The nucleotide and amino acid sequences shown in SEQ ID NOs:1 through 72 are found, with the same SEQ ID NO, in U.S. application Ser. No. 09/931,457. Table 1B and the sequence descriptions following indicate the SEQ ID NOs in the present application.

TABLE 1B
Plant Biosynthetic Enzymes

| | | SEQ ID NO: | |
|---|---|---|---|
| Polypeptide | Clone | (Nucleotid) | (Amino Acid) |
| soybean CS | src3c.pk002.e24:fis | 73 | 74 |
| soybean CS | Contig of: | 75 | 76 |
| | sl1.pk0067.g6 (5′ SID) | | |
| | sl1.pk0076.b3:fis | | |
| soybean CS | Contig of: | 77 | 78 |
| | sdr1f.pk004.d11 (5′ SID) | | |
| | sic1c.pk002.c7 | | |
| | sr1.pk0043.d9 | | |
| | sl2.pk0005.c5 | | |
| | sdp2c.pk012.d22 | | |
| | sdp2c.pk014.d22 | | |
| | sdr1f.pk004.d11.f | | |

SEQ ID NO:79 is the sequence of primer 1 described in Example 14.

SEQ ID NO:80 is the sequence of primer 2 described in Example 14.

SEQ ID NO:81 is the O-acetylserine (thiol) lyase (OASTL) (also known as cysteine synthase) coding sequence of clone se3.05h06 (SEQ ID NO:30).

SEQ ID NO:82 is the sequence of primer 3 described in Example 15.

SEQ ID NO:83 is the sequence of primer 4 described in Example 15.

SEQ ID NO:84 is the sequence of primer 5 described in Example 16.

SEQ ID NO:85 is the sequence of primer 6 described in Example 16.

SEQ ID NO:86 is the sequence of primer 7 described in Example 17.

SEQ ID NO:87 is the sequence of primer 8 described in Example 17.

SEQ ID NO:88 is the O-acetylserine (thiol) lyase (OASTL) coding sequence of clone src3c.pk002.e24:fis (SEQ ID NO:73).

SEQ ID NO:89 is the sequence of O-acetylserine (thiol)lyase from Populus alba x Populus tremula (NCBI General Identifier No. 34099833).

SEQ ID NO:90 is the sequence of plastidic cysteine synthase 1 from Solanum tuberosum (NCBI General Identifier No. 12081919).

SEQ ID NO;91 is the sequence of SEQ ID NO:198006 in U.S. Application No. 20004031072.

SEQ ID NO:92 is the sequence of SEQ ID NO:129998 in U.S. Application No. 20004123343.

SEQ ID NO:93 is the serine acetyltransferase (SAT) amino acid sequence of clone sr1.pk0162.a9 (previously disclosed in U.S. Pat. No. 6,548,280, issued Apr. 15, 2003, and is herein incorporated by reference).

SEQ ID NO:94 is the serine acetyltransferase (SAT) amino acid sequence of clone srm.pk0021.f11 (previously disclosed in U.S. Pat. No. 6,548,280, issued Apr. 15, 2003, and is herein incorporated by reference).

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. The terms “polynucleotide,” “polynucleotide sequence,” “nucleic acid sequence,” and “nucleic acid fragment”/“isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include at least 30 contiguous nucleotides, preferably at least 40 contiguous nucleotides, most preferably at least 60 contiguous nucleotides derived from SEQ ID NOs:1, 3, 5, 42, 44, 46, 48, 50, SEQ ID NOs:8, 10, 12, 14, 16, 18, 53 and 55, SEQ ID NOs:21, 23, 25, 27, and 58, SEQ ID NOs:30, 61, and 63, and SEQ ID NOs:33, 35, 37, 39, 67, 69, and 71, or the complement of such sequences.

The terms “cysteine synthase” (CS) and “O-acetylserine (thiol) lyase” (OASTL) are used interchangeably. “O-acetylserine (thiol) lyase” (E.C. 4.2.99.8) catalyzes the formation of cysteine from O-acetylserine and hydrogen sulfide with the release of acetic acid. The last steps of cysteine biosynthesis are catalyzed by a bi-enzyme complex composed of “serine acetyltransferase” (SAT) and OASTL. “Serine acetyltransferase” catalyzes the production of O-acetylserine from serine and acetyl-coenzyme A.

The term “isolated” polynucleotide refers to a polynucleotide that is substantially free from other nucleic acid sequences with which it is normally associated such as other chromosomal and extrachromosomal DNA and RNA. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The term “recombinant” means, for example, that a nucleic acid sequence is made by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

As used herein, “substantially similar” refers to nucleic acid fragments wherein changes in one or more nucleotide bases results in substitution of one or more amino acids, but do not affect the functional properties of the polypeptide encoded by the nucleotide sequence. “Substantially similar” also refers to nucleic acid fragments wherein changes in one or more nucleotide bases does not affect the ability of the nucleic acid fragment to mediate alteration of gene expression by gene silencing through for example antisense or co-suppression technology. “Substantially similar” also refers to modifications of the nucleic acid fragments of the instant invention such as deletion or insertion of one or more nucleotides that do not substantially affect the functional properties of the resulting transcript vis-à-vis the ability to mediate gene silencing or alteration of the functional properties of the resulting protein molecule. It is therefore understood that the invention encompasses more than the specific exemplary nucleotide or amino acid sequences and includes functional equivalents thereof. The terms “substantially similar” and “corresponding substantially” are used interchangeably herein.

Substantially similar nucleic acid fragments may be selected by screening nucleic acid fragments representing subfragments or modifications of the nucleic acid fragments of the instant invention, wherein one or more nucleotides are substituted, deleted and/or inserted, for their ability to affect the level of the polypeptide encoded by the unmodified nucleic acid fragment in a plant or plant cell. For example, a substantially similar nucleic acid fragment representing at least 30 contiguous nucleotides derived from the instant nucleic acid fragment can be constructed and introduced into a plant or plant cell. The level of the polypeptide encoded by the unmodified nucleic acid fragment present in a plant or plant cell exposed to the substantially similar nucleic fragment can then be compared to the level of the polypeptide in a plant or plant cell that is not exposed to the substantially similar nucleic acid fragment.

For example, it is well known in the art that antisense suppression and co-suppression of gene expression may be accomplished using nucleic acid fragments representing less than the entire coding region of a gene, and by using nucleic acid fragments that do not share 100% sequence identity with the gene to be suppressed. Moreover, alterations in a nucleic acid fragment which result in the production of a chemically equivalent amino acid at a given site, but do not affect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Consequently, an isolated polynucleotide comprising a nucleotide sequence of at least 30 (preferably at least 40, most preferably at least 60) contiguous nucleotides derived from a nucleotide sequence selected from the group consisting of SEQ ID NOs:1, 3, 5, 42, 44, 46, 48, 50, SEQ ID NOs:8, 10, 12, 14, 16, 18, 53 and 55, SEQ ID NOs:21, 23, 25, 27, and 58, SEQ ID NOs:30, 61, and 63, and SEQ ID NOs:33, 35, 37, 39, 67, 69, and 71 and the complement of such nucleotide sequences may be used in methods of selecting an isolated polynucleotide that affects the expression of an aspartic-semialdehyde dehydrogenase, a diaminopimelate decarboxylase, a homoserine kinase, a cysteine synthase, or a cystathionine β-lyase polypeptide in a host cell. A method of selecting an isolated polynucleotide that affects the level of expression of a polypeptide in a host cell may comprise the steps of: constructing an isolated polynucleotide of the present invention or an isolated chimeric gene of the present invention; introducing the isolated polynucleotide or the isolated chimeric gene into a host cell; measuring the level of a polypeptide or enzyme activity in the host cell containing the isolated polynucleotide; and comparing the level of a polypeptide or enzyme activity in the host cell containing the isolated polynucleotide with the level of a polypeptide or enzyme activity in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Moreover, the skilled artisan recognizes that substantially similar nucleic acid sequences encompassed by this invention are also defined by their ability to hybridize, under moderately stringent conditions (for example, 0.5×SSC, 0.1% SDS, 60° C.) with the sequences exemplified herein, or to any portion of the nucleotide sequences reported herein and functional equivalents thereof. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

Substantially similar nucleic acid fragments of the instant invention may also be characterized by the percent identity of the amino acid sequences that they encode to the amino acid sequences disclosed herein, as determined by algorithms commonly employed by those skilled in this art. It is well understood by one skilled in the art that many levels of sequence identity are useful in identifying related polypeptide sequences. Useful examples of percent identities are 50%, 55%, 60%, 65%, 70%, 75%, 80%, 85%, 90%, or 95%, or any integer percentage from 55% to 100%. Suitable nucleic acid fragments (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above identities but typically encode a polypeptide having at least 50 amino acids, preferably at least 100 amino acids, more preferably at least 150 amino acids, still more preferably at least 200 amino acids, and most preferably at least 250 amino acids. Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

The “Clustal V method of alignment” corresponds to the alignment method labeled Clustal V (described by Higgins and Sharp (1989) CABIOS. 5:151-153) and found in the Megalign program of the LASERGENE bioinformatics computing suite (DNASTAR Inc., Madison, Wis.). The “default parameters” are the parameters pre-set by the manufacturer of the program and for multiple alignments they correspond to GAP PENALTY=10 and GAP LENGTH PENALTY=10, while for pairwise alignments they are KTUPLE 1, GAP PENALTY=3, WINDOW=5 and DIAGONALS SAVED=5. After alignment of the sequences, using the Clustal V program, it is possible to obtain a “percent identity” by viewing the “sequence distances” table on the same program. It is well understood by one skilled in the art that many levels of sequence identity are useful in identifying related polypeptide sequences. Useful examples of percent identities are 50%, 55%, 60%, 65%, 70%, 75%, 80%, 85%, 86%, 90%, or 95%, or any integer percentage from 55% to 100%.

A “substantial portion” of an amino acid or nucleotide sequence comprises an amino acid or a nucleotide sequence that is sufficient to afford putative identification of the protein or gene that the amino acid or nucleotide sequence comprises. Amino acid and nucleotide sequences can be evaluated either manually, by one skilled in the art, or by using computer-based sequence comparison and identification tools that employ algorithms such as BLAST (Basic Local Alignment Search Tool; Altschul et al. (1993) J. Mol. Biol. 215:403-410). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. Accordingly, a “substantial portion” of a nucleotide sequence comprises a nucleotide sequence that will afford specific identification and/or isolation of a nucleic acid fragment comprising the sequence. The instant specification teaches amino acid and nucleotide sequences encoding polypeptides that comprise one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Sequence identity” or “identity” in the context of nucleic acid or polypeptide sequences refers to the nucleic acid bases or amino acid residues in the two sequences that are the same when aligned for maximum correspondence over a specified comparision window.

Thus, “Percentage of sequence identity” refers to the valued determined by comparing two optimally aligned sequences over a comparison window, wherein the portion of the polynucleotide sequence in the comparison window may comprise additionas or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and mutliplying the results by 100 to yield the percentage of sequence identity. Useful examples of percent identities include, but are not limited to, 50%, 55%, 60%, 65%, 70%, 75%, 80%, 85%, 90%, or 95%, or any integer percentage from 55% to 100%. Indeed, any integer amino acid identity from 50%-100% may be useful in describing the present invention. Also, of interest is any full or partial complement of this isolated nucleotide fragment. These identities can be determined using any of the programs described herein.

Sequence alignments and percent identity or similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal V method of alignment (Higgins, D. G. and Sharp, P. M. (1989) Comput. Appl. Biosci. 5:151-153; Higgins, D. G. et al. (1992) Comput. Appl. Biosci. 8:189-191) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

It is well understood by one skilled in the art that many levels of sequence identity are useful in identifying polypeptides, from other plant species, wherein such polypeptides have the same or similar function or activity.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign-gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a nucleotide sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence which can stimulate promoter activity and may be an innate element of the promoter or a heterologous element inserted to enhance the level or tissue-specificity of a promoter. Promoters may be derived in their entirety from a native gene, or may be composed of different elements derived from different promoters found in nature, or may even comprise synthetic nucleotide segments. It is understood by those skilled in the art that different promoters may direct the expression of a gene in different tissues or cell types, or at different stages of development, or in response to different environmental conditions. Promoters which cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg (1989) Biochemistry of Plants 15:1-82. It is further recognized that since in most cases the exact boundaries of regulatory sequences have not been completely defined, nucleic acid fragments of different lengths may have identical promoter activity.

Any promoter useful in plant transgene expression can be used to practice the invention. Examples include, but are not limited to, a β-conglycinin promoter (the α′-subunit of β-conglycinin—referred to herein as the β-conglycinin promoter), a Kunitz soybean trypsin inhibitor (KSTI or Kti) promoter, a napin promoter, beta-phaseolin promoter, oleosin promoter, albumin promoter, a zein promoter, a Bce4 promoter, a legumin B4 promoter, a T7 promoter and a 35S promoter.

Co-suppressed plants that comprise recombinant expression constructs with the promoter of the α′-subunit of β-conglycinin will often exhibit suppression of both the α and α′ subunits of β-congylcinin (as described in PCT Publication No. WO 97/47731, which published on Dec. 18, 1997, the disclosure of which is hereby incorporated by reference).

Examples of seed-specific promoters include, but are not limited to, the promoters of seed storage proteins, which can represent up to 90% of total seed protein in many plants. The seed storage proteins are strictly regulated, being expressed almost exclusively in seeds in a highly tissue-specific and stage-specific manner (Higgins et al., Ann. Rev. Plant Physiol. 35:191-221 (1984); Goldberg et al., Cell 56:149-160 (1989)). Moreover, different seed storage proteins may be expressed at different stages of seed development.

Expression of seed-specific genes has been studied in great detail (See reviews by Goldberg et al., Cell 56:149-160 (1989) and Higgins et al., Ann. Rev. Plant Physiol. 35:191-221 (1984)). There are currently numerous examples of seed-specific expression of seed storage protein genes in transgenic dicotyledonous plants. These include genes from dicotyledonous plants for bean β-phaseolin (Sengupta-Gopalan et al., Proc. Natl. Acad. Sci. USA 82:3320-3324 (1985); Hoffman et al., Plant Mol. Biol. 11:717-729 (1988)), bean lectin (Voelker et al., EMBO J. 6:3571-3577 (1987)), soybean lectin (Okamuro et al., Proc. Natl. Acad. Sci. USA 83:8240-8244 (1986)), soybean Kunitz trypsin inhibitor (Perez-Grau et al., Plant Cell 1:1095-1109 (1989)), soybean β-conglycinin (Beachy et al., EMBO J. 4:3047-3053 (1985); pea vicilin (Higgins et al., Plant Mol. Biol. 11:683-695 (1988)), pea convicilin (Newbigin et al., Planta 180:461-470 (1990)), pea legumin (Shirsat et al., Mol. Gen. Genetics 215:326-331 (1989)); rapeseed napin (Radke et al., Theor. Appl. Genet. 75:685-694 (1988)) as well as genes from monocotyledonous plants such as for maize 15 kD zein (Hoffman et al., EMBO J. 6:3213-3221 (1987)), maize 18 kD oleosin (Lee at al., Proc. Natl. Acad. Sci. USA 88:6181-6185 (1991)), barley β-hordein (Marris et al., Plant Mol. Biol. 10:359-366 (1988)) and wheat glutenin (Colot et al., EMBO J. 6:3559-3564 (1987)). Moreover, promoters of seed-specific genes operably linked to heterologous coding sequences in chimeric gene constructs also maintain their temporal and spatial expression pattern in transgenic plants. Such examples include use of Arabidopsis thaliana 2S seed storage protein gene promoter to express enkephalin peptides in Arabidopsis and Brassica napus seeds (Vandekerckhove et al., Bio/Technology 7:929-932 (1989)), bean lectin and bean β-phaseolin promoters to express luciferase (Riggs et al., Plant Sci. 63:47-57 (1989)), and wheat glutenin promoters to express chloramphenicol acetyl transferase (Colot et al., EMBO J. 6:3559-3564 (1987)).

As was noted above, any type of promoter such as constitutive, tissue-preferred, inducible promoters can be used to practice the invention. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the GRP1-8 promoter and other transcription initiation regions from various plant genes known to those of skill.

Examples of inducible promoters are the Adh1 promoter which is inducible by hypoxia or cold stress, the Hsp70 promoter which is inducible by heat stress, and the PPDK promoter which is inducible by light. Also useful are chemical-inducible promoters whose transcriptional activity is regulated by the presence or absence of alcohol, tetracycline, steroids (i.e., ecdysone; U.S. Pat. No. 6,379,945), metals and other compounds.

Examples of promoters under developmental control include promoters that initiate transcription preferentially in certain tissues, such as leaves, roots, fruit, seeds, or flowers. One such example is the RuBis Co promoter. Another exemplary promoter is the anther specific promoter 5126 (U.S. Pat. Nos. 5,689,049 and 5,689,051). In addition to those mentioned above, other examples of seed-specific promoters include, but are not limited to, 27 kD gamma zein promoter and waxy promoter (Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47:95-102 (1986) and Reina, M., Ponte, I., Guillen, P., Boronat, A. and Palau, J., Sequence analysis of a genomic clone encoding a Zc2 protein from Zea mays W64 A, Nucleic Acids Res. 18(21):6426 (1990)). See the following reference relating to the waxy promoter: Kloesgen, R. B., Gierl, A., Schwarz-Sommer, Z. S. and Saedler, H., Molecular analysis of the waxy locus of Zea mays, Mol. Gen. Genet. 203:237-244 (1986). Promoters that express in the embryo, pericarp, and endosperm are disclosed in PCT Publication No. WO 00/11177, which published Mar. 2, 2000, and PCT Publication No. WO 00/12733, which published Mar. 9, 2000. The disclosures of each of these are incorporated herein by reference in their entirety.

Either heterologous or non-heterologous (i.e., endogenous) promoters can be used to practice the invention.

The promoter is then operably linked using conventional means well known to those skilled in the art to a DNA sequence which, when expressed by a host produces an RNA meeting certain criteria.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Mol. Biotechnol. 3:225-236).

“3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into polypeptides by the cell. “cDNA” refers to DNA that is complementary to and derived from an mRNA template. The cDNA can be single-stranded or converted to double stranded form using, for example, the Klenow fragment of DNA polymerase 1. “Sense-RNA” refers to an RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065, incorporated herein by reference). The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” refers to the association of two or more nucleic acid fragments on a single polynucleotide so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “endogenous nucleotide sequence” refers to any nucleotide sequence which is present in the genome of the host prior to transformation with the recombinant construct of the present invention, whether naturally-occurring or non-naturally occurring, i.e., introduced by recombinant means, mutagenesis, etc.

The term “non-naturally occurring” means artificial, not consistent with what is normally found in nature.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

Cosuppression technology constitutes the subject matter of U.S. Pat. No. 5,231,020, which issued to Jorgensen et al. on Jul. 27, 1999. The phenomenon observed by Napoli et al. in petunia was referred to as “cosuppression” since expression of both the endogenous gene and the introduced transgene were suppressed (for reviews see Vaucheret et al., Plant J. 16:651-659 (1998); and Gura, Nature 404:804-808 (2000)).

Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al. (1998) Plant J 16:651-659; and Gura (2000) Nature 404:804-808). The overall efficiency of this phenomenon is low, and the extent of the RNA reduction is widely variable. Recent work has described the use of “hairpin” structures that incorporate all, or part, of an mRNA encoding sequence in a complementary orientation that results in a potential “stem-loop” structure for the expressed RNA (PCT Publication WO 99/53050 published on Oct. 21, 1999). This increases the frequency of co-suppression in the recovered transgenic plants. Another variation describes the use of plant viral sequences to direct the suppression, or “silencing”, of proximal mRNA encoding sequences (PCT Publication WO 98/36083 published on Aug. 20, 1998). Both of these co-suppressing phenomena have not been elucidated mechanistically, although recent genetic evidence has begun to unravel this complex situation (Elmayan et al. (1998) Plant Cell 10:1747-1757).

In addition to cosuppression, antisense technology has also been used to block the function of specific genes in cells. Antisense RNA is complementary to the normally expressed RNA, and presumably inhibits gene expression by interacting with the normal RNA strand. The mechanisms by which the expression of a specific gene are inhibited by either antisense or sense RNA are on their way to being understood. However, the frequencies of obtaining the desired phenotype in a transgenic plant may vary with the design of the construct, the gene, the strength and specificity of its promoter, the method of transformation and the complexity of transgene insertion events (Baulcombe, Curr. Biol. 12(3):R82-84 (2002); Tang et al., Genes Dev. 17(1):49-63 (2003); Yu et al., Plant Cell. Rep. 22(3):167-174 (2003)). Cosuppression and antisense inhibition are also referred to as “gene silencing”, “post-transcriptional gene silencing” (PTGS), RNA interference or RNAi. See for example U.S. Pat. No. 6,506,559.

The term “expression”, as used herein, refers to the production of a functional end-product, be it mRNA or translation of mRNA into a polypeptide. “Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020).

“Overexpression” refers to the production of a functional end-product in transgenic organisms that exceeds levels of production when compared to expression of that functional end-product in a normal, wild type or non-transformed organism.

A “protein” or “polypeptide” is a chain of amino acids arranged in a specific order determined by the coding sequence in a polynucleotide encoding the polypeptide. Each protein or polypeptide has a unique function.

“Altered levels” or “altered expression” refers to the production of gene product(s) in transgenic organisms in amounts or proportions that differ from that of normal or non-transformed organisms.

“Mature protein” or the term “mature” when used in describing a protein refers to a post-translationally processed polypeptide; i.e., one from which any pre- or propeptides present in the primary translation product have been removed. “Precursor protein” or the term “precursor” when used in describing a protein refers to the primary product of translation of mRNA; i.e., with pre- and propeptides still present. Pre- and propeptides may be but are not limited to intracellular localization signals.

“Stable transformation” refers to the transfer of a nucleic acid fragment into a genome of a host organism, including either nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The preferred method of cell transformation of rice, corn and other monocots is the use of particle-accelerated or “gene gun” transformation technology (Klein et al., Nature (London) 327:70-73 (1987); U.S. Pat. No. 4,945,050), or an Agrobacterium-mediated method using an appropriate Ti plasmid containing the transgene (Ishida Y. et al., Nature Biotech. 14:745-750 (1996)).

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Maniatis”).

“PCR” or “polymerase chain reaction” is well known by those skilled in the art as a technique used for the amplification of specific DNA segments (U.S. Pat. Nos. 4,683,195 and 4,800,159).

The terms “recombinant construct”, “expression construct” and “recombinant expression construct” are used interchangeably herein. These terms refer to a functional unit of genetic material that can be inserted into the genome of a cell using standard methodology well known to one skilled in the art. Such construct may be itself or may be used in conjunction with a vector. If a vector is used then the choice of vector is dependent upon the method that will be used to transform host plants as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

The regeneration, development, and cultivation of plants from single plant protoplast transformants or from various transformed explants is well known in the art (Weissbach and Weissbach, In: Methods for Plant Molecular Biology, (Eds.), Academic Press, Inc. San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous isolated nucleic acid fragment that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue.

The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011, McCabe et. al., Bio/Technology 6:923 (1988), Christou et al., Plant Physiol. 87:671-674 (1988)); Brassica (U.S. Pat. No. 5,463,174); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258, (1995)).

Transformation of monocotyledons using electroporation, particle bombardment, and Agrobacterium have also been reported. Transformation and plant regeneration have been achieved in asparagus (Bytebier et al., Proc. Natl. Acad. Sci. (USA) 84:5354, (1987)); barley (Wan and Lemaux, Plant Physiol 104:37 (1994)); Zea mays (Rhodes et al., Science 240:204 (1988), Gordon-Kamm et al., Plant Cell 2:603-618 (1990), Fromm et al., Bio/Technology 8:833 (1990), Koziel et al., Bio/Technology 11: 194, (1993), Armstrong et al., Crop Science 35:550-557 (1995)); oat (Somers et al., Bio/Technology 10: 15 89 (1992)); orchard grass (Horn et al., Plant Cell Rep. 7:469 (1988)); rice (Toriyama et al., Theor Appl. Genet. 205:34, (1986); Part et al., Plant Mol. Biol. 32:1135-1148, (1996); Abedinia et al., Aust. J. Plant Physiol. 24:133-141 (1997); Zhang and Wu, Theor. Appl. Genet. 76:835 (1988); Zhang et al. Plant Cell Rep. 7:379, (1988); Battraw and Hall, Plant Sci. 86:191-202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)), and wheat (Vasil et al., Bio/Technology 10:667 (1992); U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)).

Transient expression systems may be used to functionally dissect isolated nucleic acid fragment constructs (see generally, Maliga et al., Methods in Plant Molecular Biology, Cold Spring Harbor Press (1995)). It is understood that any of the nucleic acid molecules of the present invention can be introduced into a plant cell in a permanent or transient manner in combination with other genetic elements such as vectors, promoters, enhancers etc.

The present invention concerns isolated polynucleotides comprising a nucleotide sequence selected from the group consisting of: (a) a nucleotide sequence encoding a polypeptide of at least 60 amino acids having at least 80% identity based on the Clustal method of alignment when compared to a polypeptide selected from the group consisting of SEQ ID NOs:2, 4, 6, 43, 45, 47, 49, and 51; (b) a nucleotide sequence encoding a polypeptide of at least 60 amino acids having at least 95% identity based on the Clustal method of alignment when compared to a polypeptide selected from the group consisting of SEQ ID NOs:9, 11, 13, 15, 17, 19, 54 and 56; (c) a nucleotide sequence encoding a polypeptide of at least 60 amino acids having at least 80% identity based on the Clustal method of alignment when compared to a polypeptide selected from the group consisting of SEQ ID NOs:22, 24, 26, 28, and 59; (d) a nucleotide sequence encoding a polypeptide of at least 60 amino acids having at least 95% identity based on the Clustal method of alignment when compared to a polypeptide selected from the group consisting of SEQ ID NOs:31, 62, and 64; and (e) a nucleotide sequence encoding a polypeptide of at least 60 amino acids having at least 85% identity based on the Clustal method of alignment when compared to a polypeptide selected from the group consisting of SEQ ID NOs:34, 36, 38, 40, 68, 70, and 72. It is preferred that the identity be at least 85%, it is preferable if the identity is at least 90%, it is more preferred that the identity be at least 95%. This invention also relates to the isolated complement of such polynucleotides, wherein the complement and the polynucleotide consist of the same number of nucleotides, and the nucleotide sequences of the complement and the polynucleotide have 100% complementarity.

Preferably, the isolated polynucleotide of the claimed invention comprises a nucleotide sequence selected from the group consisting of SEQ ID NOs:1, 3, 5, 42, 44, 46, 48, 50, 8, 10, 12, 14, 16, 18, 53, 55, 21, 23, 25, 27, 58, 30, 61, 63, 33, 35, 37, 39, 67, 69, and 71.

In another aspect the present invention concerns an isolated polynucleotide sequence comprising:
(a) a nucleotide sequence encoding a polypeptide having cysteine synthase activity, wherein the polypeptide has an amino acid sequence of at least 90% sequence identity, based on the Clustal method of alignment, when compared to one of SEQ ID NO:74, 76 or 78;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

In still another aspect, the present invention concerns an isolated polynucleotide sequence comprising:
(a) a nucleotide sequence encoding a polypeptide having cysteine synthase activity, wherein the polypeptide has an amino acid sequence of greater than 87.1% sequence identity, based on the Clustal method of alignment, when compared to SEQ ID NO:31;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

Nucleic acid fragments encoding at least a portion of several plant amino acid biosynthetic enzymes have been isolated and identified by comparison of random plant cDNA sequences to public databases containing nucleotide and protein sequences using the BLAST algorithms well known to those skilled in the art. The nucleic acid fragments of the instant invention may be used to isolate cDNAs and genes encoding homologous proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other aspartic semialdehyde dehydrogenases, diaminopimelate decarboxylases, homoserine kinases, cysteine synthases or cystathionine β-lyases, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, an entire sequence can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al. (1988) Proc. Natl. Acad. Sci. USA 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al. (1989) Proc. Natl. Acad. Sci. USA 86:5673-5677; Loh et al. (1989) Science 243:217-220). Products generated by the 3′ and 5′ RACE procedures can be combined to generate full-length cDNAs (Frohman and Martin (1989) Techniques 1:165). Consequently, a polynucleotide comprising a nucleotide sequence of at least 30 (preferably at least 40, most preferably at least 60) contiguous nucleotides derived from a nucleotide sequence selected from the group consisting of SEQ ID NOs:1, 3, 5, 42, 44, 46, 48, 50, 8, 10, 12, 14, 16, 18, 59, 61, 21, 23, 25, 27, 64, 30, 33, 35, 37, 39, 53, 55, and 57 and the complement of such nucleotide sequences may be used in such methods to obtain a nucleic acid fragment encoding a substantial portion of an amino acid sequence of a polypeptide.

The present invention relates to a method of obtaining a nucleic acid fragment encoding a substantial portion of an aspartic semialdehyde dehydrogenase, diaminopimelate decarboxylase, homoserine kinase, cysteine synthase, or cystathionine β-lyase polypeptide, preferably a substantial portion of a plant aspartic semialdehyde dehydrogenase, diaminopimelate decarboxylase, homoserine kinase, cysteine synthase, or cystathionine β-lyase polypeptide, comprising the steps of: synthesizing an oligonucleotide primer comprising a nucleotide sequence of at least 30 (preferably at least 40, most preferably at least 60) contiguous nucleotides derived from a nucleotide sequence selected from the group consisting of SEQ ID NOs:1, 3, 5, 42, 44, 46, 48, 50, 8, 10, 12, 14, 16, 18, 53, 55, 21, 23, 25, 27, 58, 30, 61, 63, 33, 35, 37, 39, 67, 69, and 71, and the complement of such nucleotide sequences; and amplifying a nucleic acid fragment (preferably a cDNA inserted in a cloning vector) using the oligonucleotide primer. The amplified nucleic acid fragment preferably will encode a portion of an aspartic semialdehyde dehydrogenase, diaminopimelate decarboxylase, homoserine kinase, cysteine synthase, or cystathionine β-lyase polypeptide.

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner (1984) Adv. Immunol. 36:1-34; Maniatis).

In another embodiment, this invention concerns viruses and host cells comprising either the chimeric genes of the invention as described herein or an isolated polynucleotide of the invention as described herein. Examples of host cells which can be used to practice the invention include, but are not limited to, yeast, bacteria, and plants.

As was noted above, the nucleic acid fragments of the instant invention may be used to create transgenic plants in which the disclosed polypeptides are present at higher or lower levels than normal or in cell types or developmental stages in which they are not normally found. This would have the effect of altering the level of free amino acids in those cells. Specifically, the enzymes of the present invention form part of the pathway towards the biosynthesis of lysine, threonine, methionine, cysteine and isoleucine. In particular, altering the level and/or function of cystathionine β-lyase will result in changes in the rate of methionine biosynthesis. Altering the level and/or function of diaminopimelate decarboxylase will result in changes in the rate of lysine biosynthesis. Altering the level and/or function of aspartate-semialdehyde dehydrogenase will result in changes in the lysine, methionine, or threonine content, especially in wheat. Altering the level of cysteine synthase will result in changes in the rate of cysteine and/or methionine biosynthesis; using this gene it will also be possible to control sulfur metabolism. Altering the level of homoserine kinase may be used to regulate threonine and methionine levels. Polypeptides encoding at least a portion of aspartic semialdehyde dehydrogenase, diaminopimelate decarboxylase, homoserine kinase, cysteine synthase, or cystathionine β-lyase may also be used in herbicide identification and design.

Overexpression of the proteins of the instant invention may be accomplished by first constructing a chimeric gene in which the coding region is operably linked to a promoter capable of directing expression of a gene in the desired tissues at the desired stage of development. The chimeric gene may comprise promoter sequences and translation leader sequences derived from the same genes. 3′ Non-coding sequences encoding transcription termination signals may also be provided. The instant chimeric gene may also comprise one or more introns in order to facilitate gene expression.

Plasmid vectors comprising the instant isolated polynucleotide (or chimeric gene) may be constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the instant polypeptides to different cellular compartments, or to facilitate its secretion from the cell. It is thus envisioned that the chimeric gene described above may be further supplemented by directing the coding sequence to encode the instant polypeptides with appropriate intracellular targeting sequences such as transit sequences (Keegstra (1989) Cell 56:247-253), signal sequences or sequences encoding endoplasmic reticulum localization (Chrispeels (1991) Ann. Rev. Plant Phys. Plant Mol. Biol. 42:21-53), or nuclear localization signals (Raikhel (1992) Plant Phys. 100:1627-1632) with or without removing targeting sequences that are already present. While the references cited give examples of each of these, the list is not exhaustive and more targeting signals of use may be discovered in the future.

It may also be desirable to reduce or eliminate expression of genes encoding the instant polypeptides in plants for some applications. In order to accomplish this, a chimeric gene designed for co-suppression of the instant polypeptide can be constructed by linking a gene or gene fragment encoding that polypeptide to plant promoter sequences. Alternatively, a chimeric gene designed to express antisense RNA for all or part of the instant nucleic acid fragment can be constructed by linking the gene or gene fragment in reverse orientation to plant promoter sequences. Either the co-suppression or antisense chimeric genes could be introduced into plants via transformation wherein expression of the corresponding endogenous genes are reduced or eliminated.

Molecular genetic solutions to the generation of plants with altered gene expression have a decided advantage over more traditional plant breeding approaches. Changes in plant phenotypes can be produced by specifically inhibiting expression of one or more genes by antisense inhibition or cosuppression (U.S. Pat. Nos. 5,190,931, 5,107,065 and 5,283,323). An antisense or cosuppression construct would act as a dominant negative regulator of gene activity. While conventional mutations can yield negative regulation of gene activity these effects are most likely recessive. The dominant negative regulation available with a transgenic approach may be advantageous from a breeding perspective. In addition, the ability to restrict the expression of a specific phenotype to the reproductive tissues of the plant by the use of tissue specific promoters may confer agronomic advantages relative to conventional mutations which may have an effect in all tissues in which a mutant gene is ordinarily expressed.

The person skilled in the art will know that special considerations are associated with the use of antisense or cosuppression technologies in order to reduce expression of particular genes. For example, the proper level of expression of sense or antisense genes may require the use of different chimeric genes utilizing different regulatory elements known to the skilled artisan. Once transgenic plants are obtained by one of the methods described above, it will be necessary to screen individual transgenics for those that most effectively display the desired phenotype. Accordingly, the skilled artisan will develop methods for screening large numbers of transformants. The nature of these screens will generally be chosen on practical grounds. For example, one can screen by looking for changes in gene expression by using antibodies specific for the protein encoded by the gene being suppressed, or one could establish assays that specifically measure enzyme activity. A preferred method will be one which allows large numbers of samples to be processed rapidly, since it will be expected that a large number of transformants will be negative for the desired phenotype.

In another embodiment, the present invention concerns an aspartic-semialdehyde dehydrogenase polypeptide of at least 50 amino acids comprising at least 70% identity based on the Clustal method of alignment compared to a polypeptide selected from the group consisting of SEQ ID NOs:2, 4, 6, 43, 45, 47, 49, and 51, a diaminopimelate decarboxylase polypeptide of at least 60 amino acids comprising at least 95% identity based on the Clustal method of alignment compared to a polypeptide selected from the group consisting of SEQ ID NOs:9, 11, 13, 15, 17, 19, 60, and 62, a homoserine kinase polypeptide of at least 60 amino acids comprising at least 70% identity based on the Clustal method of alignment compared to a polypeptide selected from the group consisting of SEQ ID NOs:22, 24, 26, 28, and 65, a cysteine synthase polypeptide of at least 60 amino acids comprising at least 90% identity based on the Clustal method of alignment compared to a polypeptide of SEQ ID NO:31, or a cystathionine β-lyase polypeptide of at least 60 amino acids comprising at least 85% identity based on the Clustal method of alignment compared to a polypeptide selected from the group consisting of SEQ ID NOs:34, 36, 38, 40, 54, 56, and 58.

The instant polypeptides (or portions thereof) may be produced in heterologous host cells, particularly in the cells of microbial hosts, and can be used to prepare antibodies to these proteins by methods well known to those skilled in the art. The antibodies are useful for detecting the polypeptides of the instant invention in situ in cells or in vitro in cell extracts. Preferred heterologous host cells for production of the instant polypeptides are microbial hosts. Microbial expression systems and expression vectors containing regulatory sequences that direct high level expression of foreign proteins are well known to those skilled in the art. Any of these could be used to construct a chimeric gene for production of the instant polypeptides. This chimeric gene could then be introduced into appropriate microorganisms via transformation to provide high level expression of the encoded plant biosynthetic enzymes. An example of a vector for high level expression of the instant polypeptides in a bacterial host is provided (Example 10).

Additionally, the instant polypeptides can be used as a target to facilitate design and/or identification of inhibitors of those enzymes that may be useful as herbicides. This is desirable because the polypeptides described herein catalyze various steps in a pathway leading to production of several essential amino acids. Accordingly, inhibition of the activity of one or more of the enzymes described herein could lead to inhibition of plant growth. Thus, the instant polypeptides could be appropriate for new herbicide discovery and design.

All or a substantial portion of the polynucleotides of the instant invention may also be used as probes for genetically and physically mapping the genes that they are a part of, and used as markers for traits linked to those genes. Such information may be useful in plant breeding in order to develop lines with desired phenotypes. For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et al. (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask (1991) Trends Genet. 7:149-154). Although current methods of FISH mapping favor use of large clones (several to several hundred KB; see Laan et al. (1995) Genome Res. 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer (1989) Proc. Natl. Acad. Sci USA 86:9402-9406; Koes et al. (1995) Proc. Natl. Acad. Sci USA 92:8149-8153; Bensen et al. (1995) Plant Cell 7:75-84). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the instant polypeptides. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding the instant polypeptides can be identified and obtained. This mutant plant can then be used to determine or confirm the natural function of the instant polypeptides disclosed herein.

The recombinant DNA constructs of the invention can be used in a method of altering the level of sulfur-containing amino acids in a soybean plant which comprises:
(a) transforming a soybean plant cell with a recombinant DNA construct of the invention;
(b) regenerating a soybean plant from the transformed plant cell of step (a); and
(c) comparing the sulfur-containing amino acid levels of a transformed soybean plant with the sulfur-containing amino acid levels of an untransformed soybean plant.

A plant transformed with a recombinant DNA construct of the invention can be used in a method for producing a soybean plant having an altered level of sulfur-containing amino acids which comprises:
(a) crossing an agronomically elite soybean line with a plant transformed with a recombinant DNA construct of the invention; and
(b) screening the seed of progeny plants obtained from step (a) for an altered level of sulfur-containing amino acids compared to an untransformed soybean plant.

A plant transformed with a recombinant DNA construct of the invention can also be used in a method for producing a soybean protein product having an altered level of sulfur-containing amino acids which comprises:
(a) crossing an agronomically elite soybean line with with a plant transformed with a recombinant DNA construct of the invention; and
(b) screening the seed of progeny plants obtained from step (a) for an altered level of sulfur-containing amino acids compared to an untransformed soybean plant; and
(c) processing the seed selected in step (b) to obtain the desired soybean protein product.

Also of interest is a method of increasing the level of sulfur-containing amino acids in a soybean plant which comprises:
(a) transforming a soybean plant cell with a recombinant DNA construct comprising an isolated polynucleotide sequence comprising:
   (i) a nucleotide sequence encoding a polypeptide having cysteine synthase activity, wherein the polypeptide has an amino acid sequence of at least 90% sequence identity, based on the Clustal method of alignment, when compared to one of SEQ ID NO:74;
   (ii) the full-length complement of the nucleotide sequence of (i); or
   (iii) all or part of a coding or non-coding region of the isolated polynucleotide sequence comprising any of the nucleotide sequences of (i) or (ii) for use in root specific co-suppression or antisense suppression of endogenous nucleic acid sequences encoding polypeptides having cysteine synthase activity
   wherein said isolated polynucleotide is operably linked to at least one regulatory sequence, said regulatory sequence being a root specific promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

Another method of interest is a method of increasing the level of sulfur-containing amino acids in a soybean plant which comprises:
(a) transforming a soybean plant cell with a recombinant DNA construct comprising an isolated polynucleotide sequence comprising:
   (i) a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31;
   for use in overexpressing endogenous nucleic acid sequences encoding polypeptides having cysteine synthase activity in the transformed plant
wherein said isolated polynucleotide is operably linked to at least one regulatory sequence, said regulatory sequence being a constitutive promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

Still another method of the invention is a method of increasing the level of sulfur-containing amino acids in a soybean plant which comprises:
(a) transforming a soybean plant cell with a recombinant DNA construct comprising an isolated polynucleotide sequence comprising a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31; for use in seed-specific overexpression of endogenous nucleic acid sequences encoding polypeptides having cysteine synthase activity
   wherein said isolated polynucleotide is operably linked to at least one regulatory sequence, said regulatory sequence being a seed-specific promoter.
(b) regenerating a soybean plant from the transformed plant cell of step (a); and
(c) comparing the sulfur-containing amino acid levels of seeds obtained from a transformed soybean plant with the sulfur-containing amino acid levels of seeds obtained from an untransformed soybean plant.

A variety of processed vegetable protein products are produced from soybean. These range from minimally processed, defatted items such as soybean meal, grits, and flours to more highly processed items such as soy protein concentrates and soy protein isolates. In other soy protein products the oil is not extracted, full-fat soy flour for example. In addition to these processed products, there are also a number of speciality products based on traditional Oriental processes, which utilize the entire bean as the starting material. Examples include soy milk, soy sauce, tofu, natto, miso, tempeh, and yuba.

As used herein, “soybean” refers to the species Glycine max, Glycine soja, or any species that is sexually cross compatible with Glycine max. A “line” is a group of plants of similar parentage that display little or no genetic variation between individuals for a least one trait. Such lines may be created by one or more generations of self-pollination and selection, or vegetative propagation from a single parent including by tissue or cell culture techniques. “Germplasm” refers to any plant(s), line(s), or population of plants that has/have the potential to be used as parent(s) in a plant breeding program. As used herein, “PI” or “plant introduction” refers to one of many soybean germplasm lines collected and maintained by the the United States Department of Agriculture. “Agronomic performance” or “agronomics” refers to heritable crop traits such as good emergence, seedling vigor, vegetative vigor, herbicide tolerance, adequate disease tolerance and, ultimately, high seed yield. “Seed yield” or “yield” refers to productivity of seeds per unit area (e.g., bushels/acre or metric tons/hectare) that a particular soybean line is capable of producing in a specific environment or generally in many environments. An “agronomically elite line” or “elite line” refers to a line with desirable agronomic performance that may or may not be used commercially. A “variety”, “cultivar”, “elite variety”, or “elite cultivar” refers to an agronomically superior elite line that has been extensively tested and is (or was) being used for commercial soybean production. “Mutation” refers to a detectable and heritable genetic change (either spontaneous or induced) not caused by segregation or genetic recombination. “Mutant” refers to an individual, or lineage of individuals, possessing a mutation. A “population” is any group of individuals that share a common gene pool. In the instant invention, this includes M1, M2, M3, M4, F1, and F2 populations. As used herein, an “M1 population” is the progeny of seeds (and resultant plants) that have been exposed to a mutagenic agent, while “M2 population” is the progeny of self-pollinated M1 plants, “M3 population” is the progeny of self-pollinated M2 plants, and “M4 population” is the progeny of self-pollinated M3 plants. As used herein, an “F1 population” is the progeny resulting from cross pollinating one line with another line. The format used herein to depict such a cross pollination is “female parent*male parent”. An “F2 population” is the progeny of the self-pollinated F1 plants. An “F2-derived line” or “F2 line” is a line resulting from the self-pollination of an individual F2 plant. An F2-derived line can be propagated through subsequent generations (F3, F4, F5 etc.) by repeated self-pollination and bulking of seed from plants of said F2-derived line. A “pedigree” denotes the parents that were crossed to produce the segregating population from which a given line was selected. For example, a pedigree of A*B for a given line C indicates A and B are the parents of C. Although lines of similar pedigree may have a trait in common (due to selection for said trait), said lines of similar pedigree may be quite different in terms of other traits. “Heritability” is a relative term referring to the extent to which a given phenotype is determined by genetic factors as opposed to environmental or analytical error factors. An “environment” is used to define a specific time, general geographical area, and climatic conditions in which soybean plants were grown to produce seeds. Within the context of this application, soybean seeds produced in a common environment were seeds that were produced on plants that were planted during the same day, within the same 1 km radius, and under similar growing conditions. Environments within this application are identified by the year and geographical site at which seeds were produced. A “heritable trait” refers to a phenotype that is largely determined by genetic factors and is relatively stable and predictable over many environments. “Heritability” does not necessarily imply that said genetic factors have been characterized. “Inheritance” refers to the actual number and nature of genes that confer a given heritable trait. For example, Mendelian segregation patterns are used to deduce the “inheritance” of a trait. “Inherent” is used to denote a plant material or seed characteristic that is conferred by the genetic makeup of the plant producing said material or seed as opposed to the environment in which the plant was grown or the way that the plant material or seed was stored or processed

“Soy protein products” are defined as those items produced from soybean seed used in feeds or foods and include, but are not limited to, those items listed in Table 1C.

TABLE 1C
Soy Protein Products Derived from Soybean Seeds^{a}

Whole Soybean Products
Roasted Soybeans
Baked Soybeans
Soy Sprouts
Soy Milk
Speciality Soy Foods/Ingredients
Soy Milk
Tofu
Tempeh
Miso
Soy Sauce
Hydrolyzed Vegetable Protein
Whipping Protein
Processed Soy Protein Products
Soybean Meal
Soy Grits
Full Fat and Defatted Flours
Soy Protein Isolates
Soy Protein Concentrates
Textured Soy Proteins
Textured Flours and Concentrates
Textured Concentrates
Textured Isolates
^{a}See Soy Protein Products: Characteristics, Nutritional Aspects and Utilization (1987). Soy Portein Council

“Processing” refers to any physical and chemical methods used to obtain the products listed in Table 1C and includes, but is not limited to heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as decribed by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [(1990) Poultry Science 69:76-83]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson, (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

General Materials and Methods:

Procedures for nucleic acid phosphorylation, restriction enzyme digests, ligation and transformation are well known in the art. Techniques suitable for use in the following examples may be found in Sambrook, J., Fritsch, E. F. and Maniatis, T., Molecular Cloning: A Laboratory Manual; Second Edition, Cold Spring Harbor Laboratory Press: Cold Spring Harbor, 1989 (hereinafter “Maniatis”).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C., 1994 or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial and plant cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Bacterial Strains and Plasmids:

E. coli TOP10 cells and E. coli electromax DH10B cells were obtained from Invitrogen (Carlsbad, Calif.). Max Efficiency competent cells of E. coli DH5α were obtained from GIBCO/BRL (Gaithersburg, Md.).

Plasmid ZBL119 has been previously described in WO2004/071467, published Aug. 26, 2004, and is herein incorporated by reference. Plasmid PHP9778 has been previously described in U.S. Pat. No. 6,555,673 B1, issued Apr. 29, 2003, and is herein incorporated by reference.

Cloning vector pCR-Script AMP SK(+) was from Stratagene (La Jolla, Calif.). Cloning vector pUC19 (Messing, J., Meth. Enzymol. 101:20 (1983)) was from New England Biolabs (Beverly, Mass.). Cloning vector pGEM-T easy was from Promega (Madison, Wis.).

Growth Conditions:

Bacterial cells were usually grown in Luria-Bertani (LB) medium containing 1% of bacto-tryptone, 0.5% of bacto-yeast extract and 1% of NaCl. Occasionally, bacterial cells were grown in SOC medium containing 2% of bacto-tryptone, 0.5% of bacto-yeast extract, 0.5% of NaCl and 20 mM glucose or in Superbroth (SB) containing 3.5% of bacto-tryptone, 2% of bacto-yeast extract, 0.05% of NaCl and 0.005 M NaOH. Antibiotics were often added to liquid or solid media in order to select for plasmids or insertions with appropriate antibiotic resistance genes. Kanamycin, ampicillin and hygromycin were routinely used at final concentrations of 50 μg/mL (Kan50), 100 μg/mL (Amp100) or 50 μg/mL (Hyg50), respectively.

### Example 1

### Composition of cDNA Libraries; Isolation and Sequencing of cDNA Clones

cDNA libraries representing mRNAs from various corn, rice, soybean, and wheat tissues were prepared. The characteristics of the libraries are described below.

TABLE 2
cDNA Libraries from Corn, Rice, Soybean, and Wheat

| Library | Tissue | Clone |
|---|---|---|
| cen1 | Corn Endosperm 12 Days After Pollination | cen1.pk0061.d4 |
| cen3n | Corn Endosperm 20 Days After Pollination* | cen3n.pk0067.a3 |
| cpe1c | Corn pooled BMS treated with chemicals related to | cpe1c.pk009.b24 |
| | phosphatase** | |
| cr1n | Corn Root From 7 Day Seedlings* | cr1n.pk0009.g4 |
| cr1n | Corn Root From 7 Day Seedlings* | cr1n.pk0103.d8 |
| p0003 | Corn Premeiotic Ear Shoot, 0.2-4 cm | p0003.cgpha22r:fis |
| p0005 | Corn Immature Ear | p0005.cbmei71r |
| p0014 | Corn Leaves 7 and 8 from Plant Transformed with | p0014.ctuui39r |
| | G-protein Gene, C. heterostrophus Resistant | |
| p0016 | Corn Tassel Shoots (0.1-1.4 cm), Pooled | p0016.ctscp83r |
| p0075 | Corn Shoot And Leaf Material From | p0075.cslab16r |
| | Dark-Grown 7 Day-Old Seedlings | |
| p0109 | Corn Leaves From Les9 Transition Zone and Les9 | p0109.cdadg47r |
| | Mature Lesions, Pooled*** | |
| p0125 | Corn Anther Prophase \|* | p0125.czaay16r |
| rca1c | Rice Nipponbare Callus | rca1c.pk005.k3 |
| rl0n | Rice Leaf 15 Days After Germination* | rl0n.pk0013.b9 |
| rlr12 | Rice Leaf 15 Days After Germination, 12 Hours After | rlr12.pk0026.g1 |
| | Infection of Strain Magaporthe grisea 4360-R-62 | |
| | (AVR2-YAMO) | |
| rlr48 | Rice Leaf 15 Days After Germination 48 Hours After | rlr48.pk0003.d12 |
| | Infection of Strain Magaporthe grisea 4360-R-62 | |
| | (AVR2-YAMO) | |
| sdp2c | Soybean Developing Pods (6-7 mm) | sdp2c.pk014.d22 |
| se3 | Soybean Embryo 13 Days After Flowering | sdp3c.pk001.o15 |
| src3c | Soybean 8 Day Old Root Infected With Cyst Nematode | src3c.pk002.e24:fis |
| sdp2c | Soybean (Glycine max L.) developing pods 6-7 mm | sdp2c.pk012.d22 |
| | | sdp2c.pk014.d22 |
| sdp3c | Soybean Developing Pods 8-9 mm | se3.05h06 |
| sdr1f | Soybean (Glycine max, Wye) 10 day old root | sdr1f.pk004.d11 (5′ |
| | | SID) |
| | | sdr1f.pk004.d11.f |
| ses8w | Mature Soybean Embryo 8 Weeks After Subculture | ses8w.pk0020.b5 |
| ses9c | Soybean Embryogenic Suspension | ses9c.pk001.a15:fis |
| sfl1 | Soybean Immature Flower | sfl1.pk0012.c4 |
| sfl1 | Soybean Immature Flower | sfl1.pk0122.f9 |
| sic1c | Soybean pooled tissue of root, stem, and leaf with iron | sic1c.pk002.c7 |
| | chlorosis conditions | |
| sl1 | Soybean Two-Week-Old Developing Seedlings | sl1.pk0067.g6 (5′ |
| | | SID) |
| | | sl1.pk0076.b3:fis |
| sl2 | Soybean (Glycine max L.) two week old developing | sl2.pk0005.c5 |
| | seedlings treated with 2.5 ppm chlorimuron | |
| sr1 | Soybean Root From 10 Day Old Seedlings | sr1.pk0043.d9 |
| | | sr1.pk0132.c1 |
| wdk1c | Wheat Developing Kernel, 3 Days After Anthesis | wdk1c.pk014.n5:fis |
| wl1n | Wheat Leaf from 7 Day Old Seedling* | wl1n.pk0065.f2 |
| wlk1 | Wheat Seedlings 1 hour After Fungicide Treatment**** | wlk1.pk0012.c2 |
| wr1 | Wheat Root From 7 Day Old Seedlings | wr1.pk0004.c11 |
| wr1 | Wheat Root From 7 Day Old Seedlings | wr1.pk0091.g6 |

*These libraries were normalized essentially as described in U.S. Pat. No. 5,482,845.
**Chemicals used included okadaic acid, cyclosporin A, calyculin A, and cypermethrin, all of which are commercially available from Molecular Biology supply sources including Calbiochem-Novabiochem Corp.
***Les9 mutants reviewed in “An update on lesion mutants” Hoisington, Maize Genetic Coop. News Lett. 60: 50-51 (1986).
****Application of 6-iodo-2-propoxy-3-propyl-4(3H)-quinazolinone; synthesis and methods of using this compound are described in U.S. Pat. No. 5,747,497, incorporated herein by reference.

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA is then used to transform DH10B electro-competent cells (Gibco BRL/Life Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones are randomly selected from each transposition reaction, plasmid DNAs are prepared via alkaline lysis, and templates are sequenced (ABI Prism dye-terminator ReadyReaction mix) outward from the transposition event site, utilizing unique primers specific to the binding sites within the transposon.

Sequence data is collected (ABI Prism Collections) and assembled using Phred/Phrap (P. Green, University of Washington, Seattle). Phrep/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (D. Gordon, University of Washington, Seattle).

### Example 2

### Identification of cDNA Clones

cDNA clones encoding plant amino acid biosynthetic enzymes were identified by conducting BLAST (Basic Local Alignment Search Tool; Altschul et al. (1993) J. Mol. Biol. 215:403-410) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

ESTs submitted for analysis are compared to the GenBank database as described above. ESTs that contain sequences more 5- or 3-prime can be found by using the BLASTn algorithm (Altschul et al (1997) Nucleic Acids Res. 25:3389-3402.) against the DuPont proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described in Example 1. Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 3

### Characterization of cDNA Clones Encoding Aspartate Semialdehyde Dehydrogenase

The BLASTX search using the EST sequences from clones listed in Table 3 revealed similarity of the polypeptides encoded by the cDNAs to aspartate semialdehyde dehydrogenase from Synechocystis sp. (DDJB Accession No. D64006; NCBI General Identifier No. 1001379) or Legionella pneumophila (GenBank Accession No. AF034213; NCBI General Identifier No. 2645882). Shown in Table 3 are the BLAST results for individual ESTs (“EST”), or for the sequences of the entire cDNA inserts comprising the indicated cDNA clones (“FIS”):

TABLE 3
BLAST Results for Sequences Encoding Polypeptides Homologous
to Aspartate Semialdehyde Dehydrogenase

| | | BLAST pLog Score | |
|---|---|---|---|
| | | Synechocystis sp. | Legionella pneumophila |
| Clone | Status | GI 1001379 | GI 2645882 |
| rlr48.pk0003.d12 | FIS | 51.00 | 36.00 |
| wr1.pk0004.c11 | EST | 67.96 | 44.74 |
| sfl1.pk0122.f9 | EST | | 6.60 |

The sequence of the entire cDNA insert in clone sfl1.pk0122.f9 was determined, RACE PCR was used to obtain the 5′ portion of the rice aspartate semialdehyde dehydrogenase, and further sequencing and searching of the DuPont proprietary database allowed the identification of a corn and other a soybean, and wheat clones encoding aspartate semialdehyde dehydrogenase. The BLASTX search using the EST sequences from clones listed in Table 4 revealed similarity of the polypeptides encoded by the cDNAs to aspartate semialdehyde dehydrogenase from Aquifex aeolicus (NCBI General Identifier No. 6225258). Shown in Table 4 are the BLAST results for the sequences of contigs assembled from two or more ESTs (“Contig”), or the sequences encoding the entire protein derived from either the entire cDNA inserts comprising the indicated cDNA clones or contigs assembled from 5′ RACE PCR and the sequence of the entire cDNA insert in the indicated cDNA clone (“CGS”):

TABLE 4
BLAST Results for Sequences Encoding Polypeptides
Homologous to Aspartate Semialdehyde Dehydrogenase

| | | BLAST pLog Score |
|---|---|---|
| Clone | Status | Aquifex aeolicus GI 6225258 |
| Contig of: | Contig | 78.70 |
| cpe1c.pk009.b24 | | |
| p0003.cgpha22r:f | | |
| is | | |
| p0016.ctscp83r | | |
| p0075.cslab16r | | |
| 5′ RACE PCR + | CGS | 89.20 |
| rlr48.pk0003.d12:fis | | |
| ses9c.pk001.a15:fis | CGS | 87.40 |
| sfl1.pk0122.f9:fis | CGS | 88.10 |
| wdk1c.pk014.n5:fis | CGS | 91.50 |

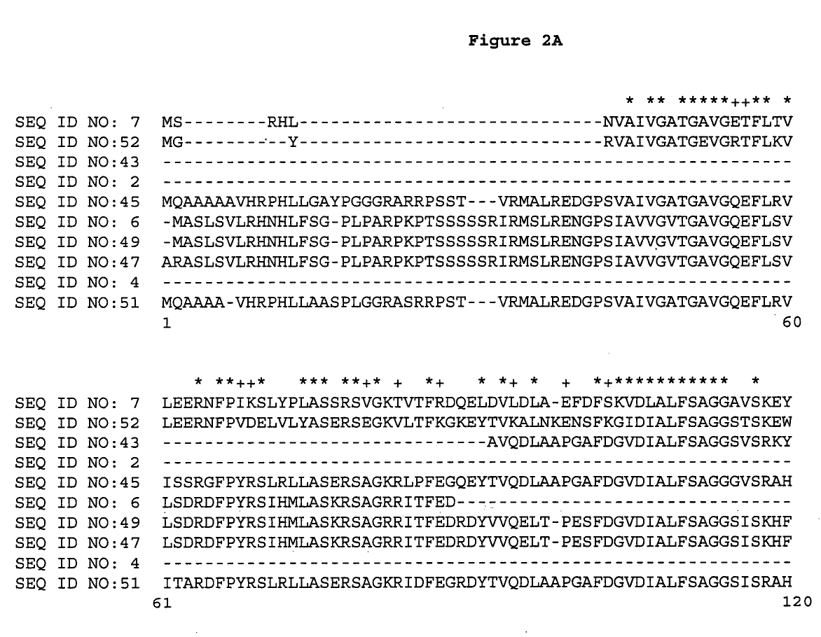

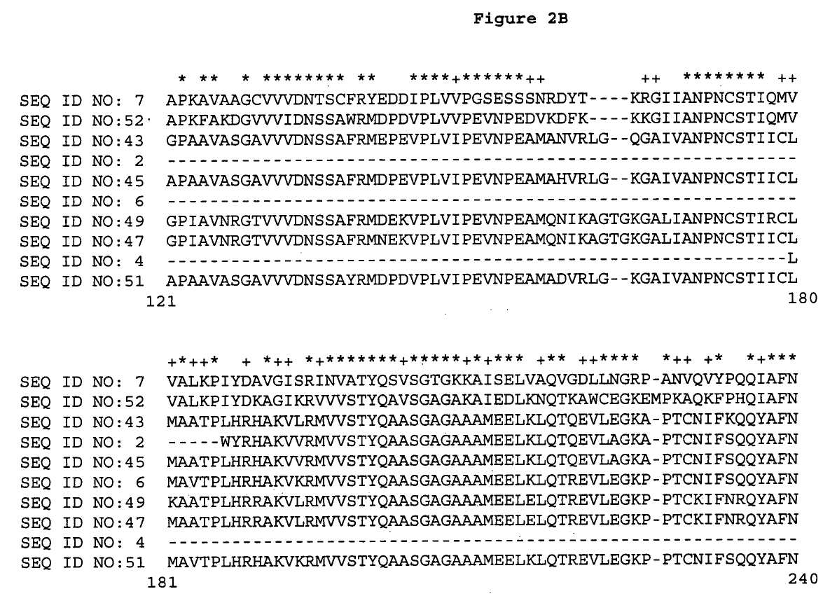

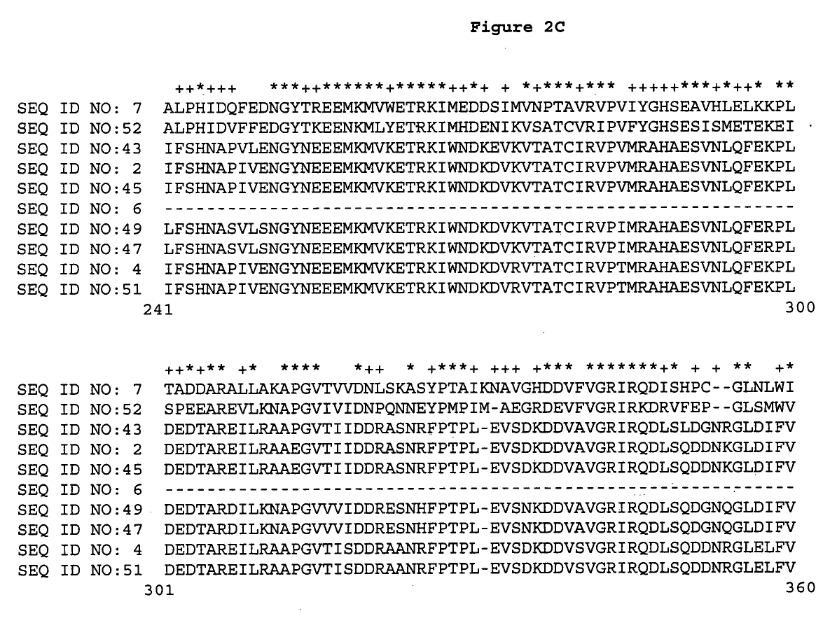

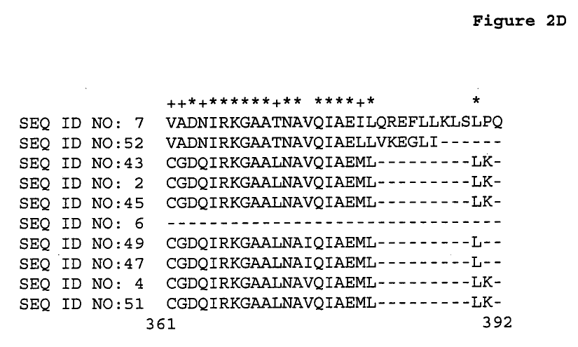

FIGS. 2A, 2B, 2C and 2D presents an alignment of the amino acid sequences set forth in SEQ ID NOs:2, 4, 6, 43, 45, 47, 49, and 51 with the Legionella pneumophila sequence (NCBI General Identifier No. 2645882; SEQ ID NO:7) and the Aquifex aeolicus sequence (NCBI General Identifier No. 6225258; SEQ ID NO:52). The data in Table 5 presents a calculation of the percent identity of the amino acid sequences set forth in SEQ ID NOs:2, 4, 6, 43, 45, 47, 49, and 51 with the Legionella pneumophila sequence (NCBI General Identifier No. 2645882; SEQ ID NO:7) and the Aquifex aeolicus sequence (NCBI General Identifier No. 6225258; SEQ ID NO:52).

TABLE 5
Percent Identity of Amino Acid Sequences Deduced From the
Nucleotide Sequences of cDNA Clones Encoding Polypeptides
Homologous to Aspartate Semialdehyde Dehydrogenase

| | |
|---|---|
| Amino Acid | Percent Identity to |

| Clone | SEQ ID NO. | 2645882 | 6225258 |
|---|---|---|---|
| rlr48.pk10003.d12 | 2 | 42.1 | 45.6 |
| wr1.pk0004.c11 | 4 | 42.3 | 44.8 |
| sfl1.pk0122.f9 | 6 | 29.1 | 25.6 |
| Contig of: | 43 | 41.2 | 45.9 |
| cpe1c.pk009.b24 | | | |
| p0003.cgpha22r:fis | | | |
| p0016.ctscp83r | | | |
| p0075.cslab16r | | | |
| 5′ RACE PCR + | 45 | 43.2 | 47.0 |
| rlr48.pk0003.d12:fis | | | |
| ses9c.pk001.a15:fis | 47 | 43.5 | 49.1 |
| sfl1.pk0122.f9:fis | 49 | 41.2 | 45.6 |
| wdk1c.pk014.n5:fis | 51 | 43.2 | 49.4 |

As seen in FIG. 2, the amino acid sequence shown in SEQ ID NO:2 is identical to amino acids 181 through 375 of SEQ ID NO:45; the sequence shown in SEQ ID NO:4 is identical to amino acids 173 through 374 of the sequence shown in SEQ ID NO:51; the sequence shown in SEQ ID NO:6 is identical to amino acids 1 through 86 of the sequence shown in SEQ ID NO:49; there are 5 amino acid differences between the sequences shown in SEQ ID NO:47 and SEQ ID NO:49; there are 18 amino acid differences between amino acids 89 through 375 of the sequence shown in SEQ ID NO:43 and the sequence shown in SEQ ID NO:45; and there are 15 differences between the amino acid sequences shown in SEQ ID NO:45 and in SEQ ID NO:51.

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of a corn aspartate semialdehyde dehydrogenase, a substantial portion and an entire rice aspartate semialdehyde dehydrogenase, a portion and an entire wheat aspartate semialdehyde dehydrogenase, and a portion and an two entire soybean aspartate semialdehyde dehydrogenases.

### Example 4

### Characterization of cDNA Clones Encoding Diaminopimelate Decarboxylase

The BLASTX search using the EST sequences from clones listed in Table 6 revealed similarity of the polypeptides encoded by the cDNAs to diaminopimelate decarboxylase from Aquifex aeolicus (GenBank Accession No. AE000728 and NCBI General Identifier No. 2983642) and Pseudomonas aeruginosa (GenBank Accession No. M23174 and NCBI General Identifier No. 118304). Shown in Table 6 are the BLAST results for individual ESTs (“EST”), the sequences of the entire cDNA inserts comprising the indicated cDNA clones (“FIS”), or the sequences of FISs encoding an entire protein (“CGS”):

TABLE 6
BLAST Results for Sequences Encoding Polypeptides
Homologous to Diaminopimelate Decarboxylase

BLAST pLog Score

| | | GI 2983642 | GI 118304 |
|---|---|---|---|
| Clone | Status | (A. aeolicus) | (P. aeruginosa) |
| cen3n.pk0067.a3 | FIS | 58.22 | 56.00 |
| cr1n.pk0103.d8 | CGS | 75.25 | 79.12 |
| rl0n.pk0013.b9 | FIS | 46.40 | 44.00 |
| sr1.pk0132.c1 | FIS | 44.70 | 39.15 |
| wlk1.pk0012.c2 | EST | 20.48 | 19.05 |

An additional soybean clone, sdp3c.pk001.o15, was identified as sharing homology with sr1.pk0132.c1. BLASTX search using the nucleotide sequences from clone sdp3c.pk001.o15 revealed similarity of the proteins encoded by the cDNA to diaminopimelate decarboxylase from Pseudomonas fluorescens (EMBO Accession No. Y12268; NCBI General Identifier No. 1929095). This EST yields a pLog value of 8.66 versus the Pseudomonas fluorescens sequence.

The sequence of the entire cDNA insert in clones sdp3c.pk001.o15 and wlk1.pk0012.c2 was determined. The BLASTX search using the EST sequences from clones listed in Table 7 revealed similarity of the polypeptides encoded by the cDNAs to diaminopimelate decarboxylase from Aquifex aeolicus (NCBI General Identifier No. 6225241) or by the Arabidopsis thaliana contig containing similarity with diaminopimelate decarboxylases (NCBI General Identifier No. 9279586). Shown in Table 7 are the BLAST results for the sequences of the entire cDNA inserts comprising the indicated cDNA clones (“FIS”), or the sequences of FISs encoding the entire protein (“CGS”):

TABLE 7
BLAST Results for Sequences Encoding Polypeptides
Homologous to Diaminopimelate Decarboxylase

| | | | BLAST |
|---|---|---|---|
| Clone | Status | Homolog | pLog Score |
| sdp3c.pk001.o15:fis | CGS | GI 6225241 (A. aeolicus) | 76.40 |
| wlk1.pk0012.c2:fis | FIS | GI 9279586 (A. thaliana) | 94.40 |

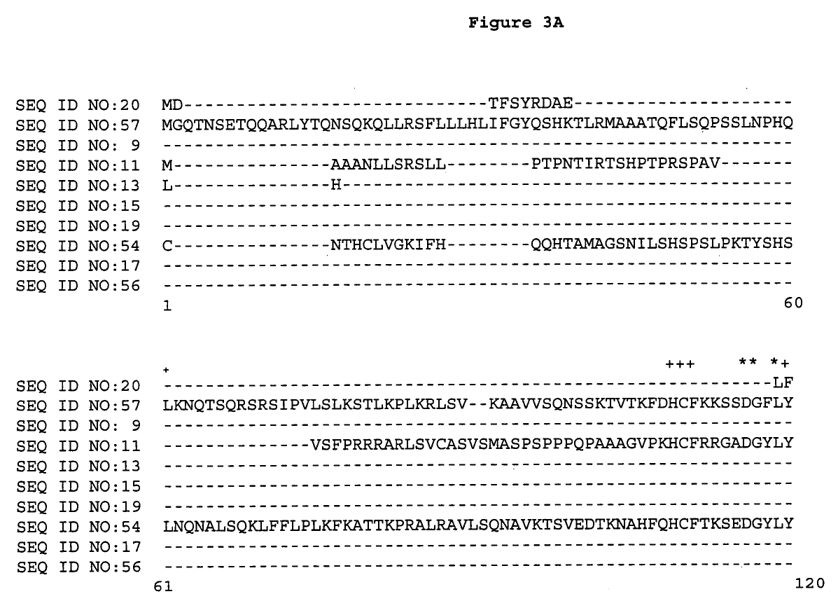

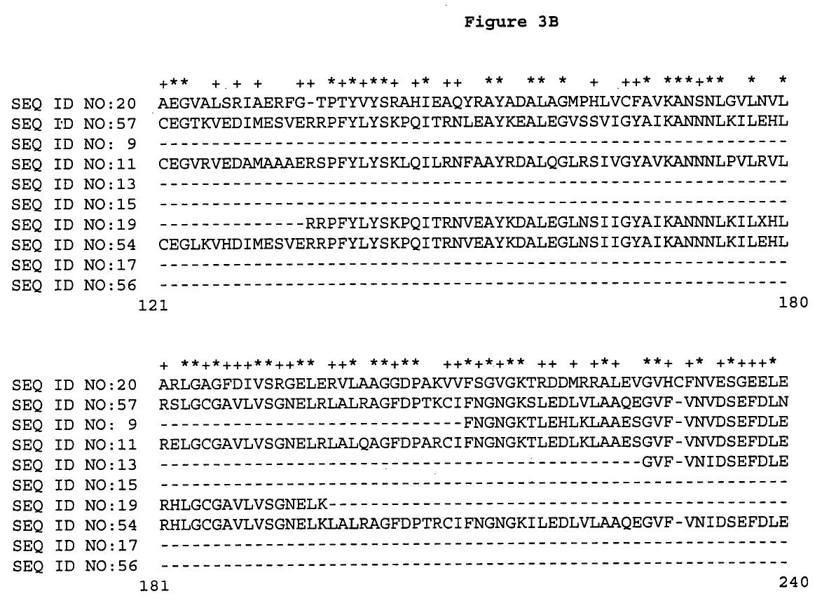

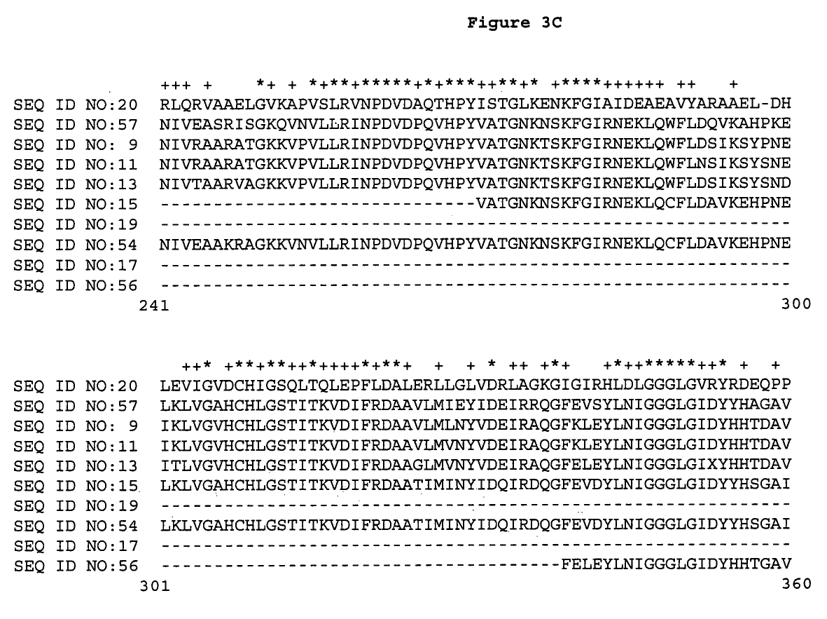

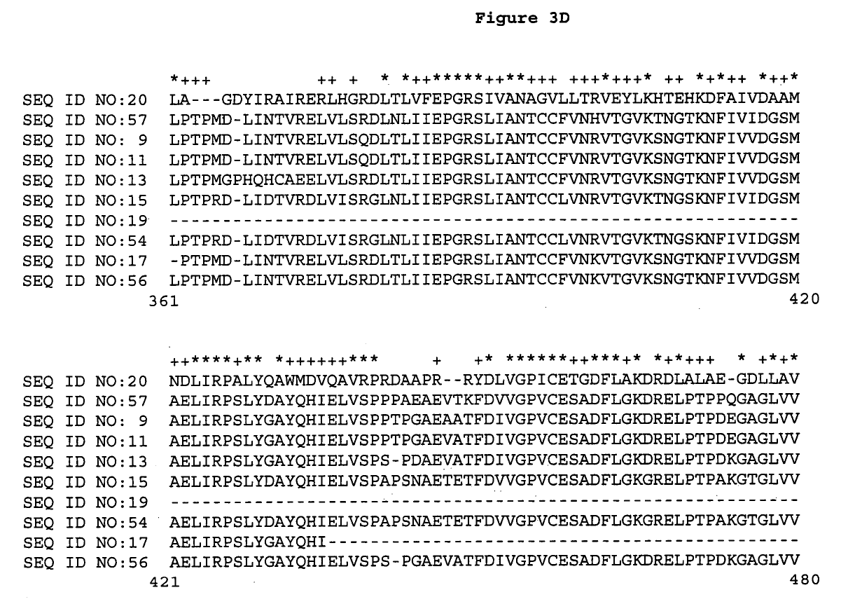

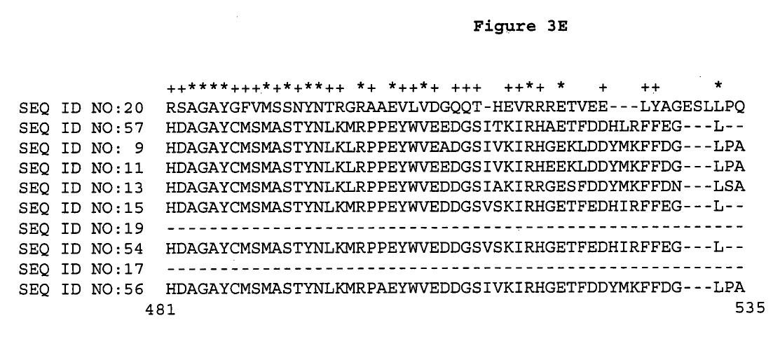

FIGS. 3A, 3B, 3C, 3D and 3E present an alignment of the amino acid sequences set forth in SEQ ID NOs:9, 11, 13, 15, 17, 19, 54, and 56 with the Pseudomonas aeruginosa sequence (NCBI General Identifier No. 118304; SEQ ID NO:20) and the Arabidopsis thaliana sequence (NCBI General Identifier No. 9279586, SEQ ID NO:57). The data in Table 8 presents a calculation of the percent identity of the amino acid sequences set forth in SEQ ID NOs:9, 11, 13, 15, 17, 19, 54, and 56 with the Pseudomonas aeruginosa sequence (NCBI General Identifier No. 118304; SEQ ID NO:20) and the Arabidopsis thaliana sequence (NCBI General Identifier No. 9279586; SEQ ID NO:57).

TABLE 8
Percent Identity of Amino Acid Sequences Deduced From
the Nucleotide Sequences of cDNA Clones Encoding
Polypeptides Homologous to Diaminopimelate Decarboxylase

| | |
|---|---|
| Amino Acid | Percent Identity to |

| Clone | SEQ ID NO. | 118304 | 9279586 |
|---|---|---|---|
| cen3n.pk0067.a3 | 9 | 34.0 | 82.2 |
| cr1n.pk0103.d8 | 11 | 35.9 | 70.6 |
| rl0n.pk0013.b9 | 13 | 32.4 | 76.8 |
| sr1.pk0132.c1 | 15 | 29.7 | 86.1 |
| wlk1.pk0012.c2 | 17 | 42.5 | 93.2 |
| sdp3c.pk001.o15 | 19 | 41.9 | 87.1 |
| sdp3c.pk001.o15:fis | 54 | 32.5 | 74.9 |
| wlk1.pk0012.c2:fis | 56 | 32. | 84.9 |

The amino acid sequence set forth in SEQ ID NO:19 is identical to amino acids 112 through 173 of the amino acid sequence set forth in SEQ ID NO:54. The amino acid sequence set forth in SEQ ID NO:17 is identical to amino acids 24 through 96 of the amino acid sequence set forth in SEQ ID NO:56.

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of one corn, one rice, two soybean and one wheat diaminopimelate decarboxylases and entire corn and soybean diaminopimelate decarboxylases.

### Example 5

### Characterization of cDNA Clones Encoding Homoserine Kinase

The BLASTX search using the EST sequences from clones listed in Table 9 revealed similarity of the polypeptides encoded by the cDNAs to homoserine kinase from Methanococcus jannaschii (GenBank Accession No. U67553 and NCBI General Identifier No. 1591748). Shown in Table 9 are the BLAST results for individual ESTs (“EST”) or for the sequences of the entire cDNA inserts comprising the indicated cDNA clones (“FIS”):

TABLE 9
BLAST Results for Sequences Encoding Polypeptides
Homologous to Homoserine Kinase

| | | BLAST pLog Score GI 1591748 |
|---|---|---|
| Clone | Status | (Methanococcus jannaschii) |
| cr1n.pk0009.g4 | FIS | 19.30 |
| rca1c.pk005.k3 | EST | 15.21 |
| ses8w.pk0020.b5 | FIS | 35.30 |
| wl1n.pk0065.f2 | EST | 5.68 |

The sequence of the entire cDNA insert in clone rca1c.pk005.k3 was determined. The BLASTX search using the EST sequences from clones listed in Table 10 revealed similarity of the polypeptides encoded by the cDNAs to homoserine kinase from Arabidopsis thaliana (NCBI General Identifier No. 4927412). Shown in Table 10 are the BLAST results for the sequences of the entire cDNA inserts comprising the indicated cDNA clone (“FIS”):

TABLE 10
BLAST Results for Sequences Encoding Polypeptides
Homologous to Homoserine Kinase

| | | BLAST pLog Score 4927412 |
|---|---|---|
| Clone | Status | (Arabidopsis thaliana) |
| rca1c.pk005.k3:fis | FIS | 88.40 |

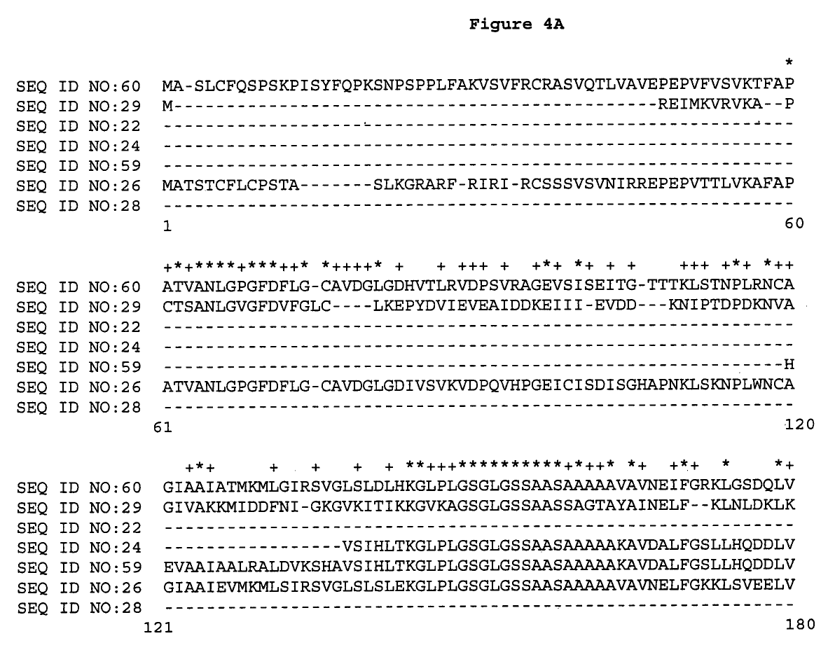

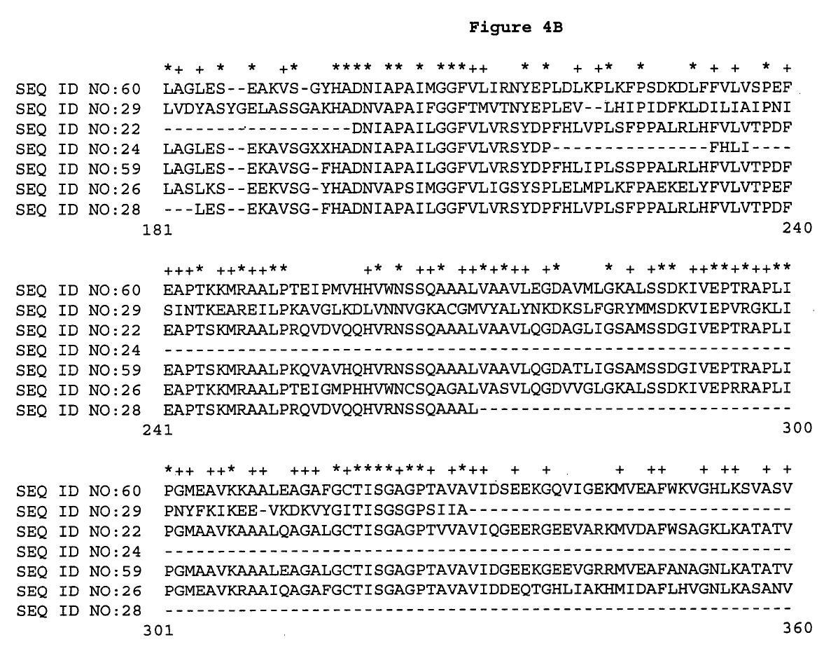

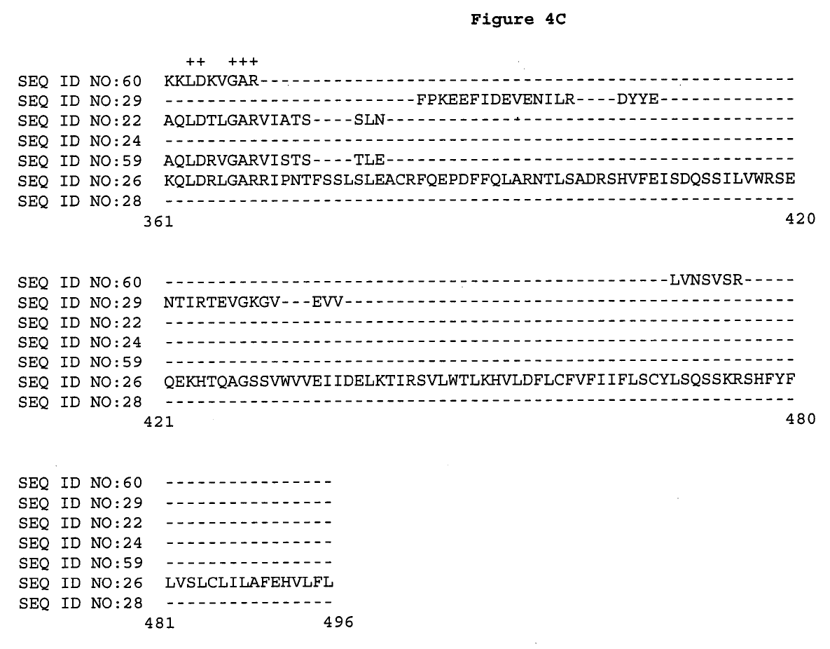

FIGS. 4A, 4B and 4C present an alignment of the amino acid sequences set forth in SEQ ID NOs:22, 24, 26, 28, and 59 with the Methanococcus jannaschii sequence (NCBI General Identifier No. 1591748; SEQ ID NO:29) and the Arabidopsis thaliana sequence (NCBI General Identifier No. 4927412; SEQ ID NO:60). The data in Table 11 presents a calculation of the percent identity of the amino acid sequences set forth in SEQ ID NOs:22, 24, 26, 28, and 59 with the Methanococcus jannaschii sequence (NCBI General Identifier No. 1591748; SEQ ID NO:29) and the Arabidopsis thaliana sequence (NCBI General Identifier No. 4927412; SEQ ID NO:60).

TABLE 11
Percent Identity of Amino Acid Sequences Deduced From
the Nucleotide Sequences of cDNA Clones Encoding
Polypeptides Homologous to Homoserine Kinase

| | SEQ | Percent Identity to | |
|---|---|---|---|
| Clone | ID NO. | NCBI GI 1591748 | NCBI GI 4927412 |
| cr1n.pk0009.g4 | 22 | 25.1 | 65.4 |
| rca1c.pk005.k3 | 24 | 48.8 | 67.1 |
| ses8w.pk0020.b5 | 26 | 28.0 | 65.7 |
| wl1n.pk0065.f2 | 28 | 29.8 | 67.9 |
| rca1c.pk005.k3:fis | 59 | 28.6 | 65.9 |

The amino acid sequence set forth in SEQ ID NO:24 is identical to amino acids 18 through 99 of the amino acid sequence set forth in SEQ ID NO:59.

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode a substantial portion of a corn and a wheat homoserine kinase, a portion and an entire rice homoserine kinase, and an entire soybean homoserine kinase.

### Example 6

### Characterization of cDNA Clones Encoding Cysteine Synthase

The BLASTX search using the EST sequences from the clone listed in Table 12 revealed similarity of the polypeptides encoded by the cDNAs to cysteine synthase from Citrullus lanatus (DDJB Accession No. D28777, NCBI General Identifier No. 540497). Shown in Table 12 are the BLAST results for the sequences of the entire cDNA inserts comprising the indicated cDNA clones encoding the entire protein (“CGS”):

TABLE 12
BLAST Results for Sequences Encoding
Polypeptides Homologous to Cysteine Synthase

| | | BLAST pLog Score |
|---|---|---|
| Clone | Status | NCBI GI 540497 (Citrullus lanatus) |
| se3.05h06 | CGS | 182.64 |

Further sequencing and searching of the DuPont proprietary database allowed the identification of corn and rice clones encoding polypeptides with similarities to cysteine synthase. The BLAST search using the sequences from clones listed in Table 13 revealed similarity of the polypeptides encoded by the cDNAs to cysteine synthase from Spinacia oleracea (NCBI General Identifier No. 416869) and Solanum tuberosum (NCBI General Identifier No. 11131628). Shown in Table 13 are the BLAST results for the sequences of the entire cDNA inserts comprising the indicated cDNA clones encoding the entire protein (“CGS”):

TABLE 13
BLAST Results for Sequences Encoding
Polypeptides Homologous to Cysteine Synthase

| | | BLAST pLog Score | |
|---|---|---|---|
| | | NCBI GI 416869 | NCBI GI 11131628 |
| Clone | Status | (Spinacia oleracea) | (Solanum tuberosum) |
| Contig of: | CGS | 158.00 | 157.00 |
| cco1n.pk083.j4 | | | |
| chp2.pk0016.b1 | | | |
| cpd1c.pk004.b20 | | | |
| cr1n.pk0083.c5 | | | |
| csi1.pk0003.g6 | | | |
| p0126.cnlcb49r | | | |
| rls6.pk0068.b7:fis | CGS | 161.00 | 163.00 |

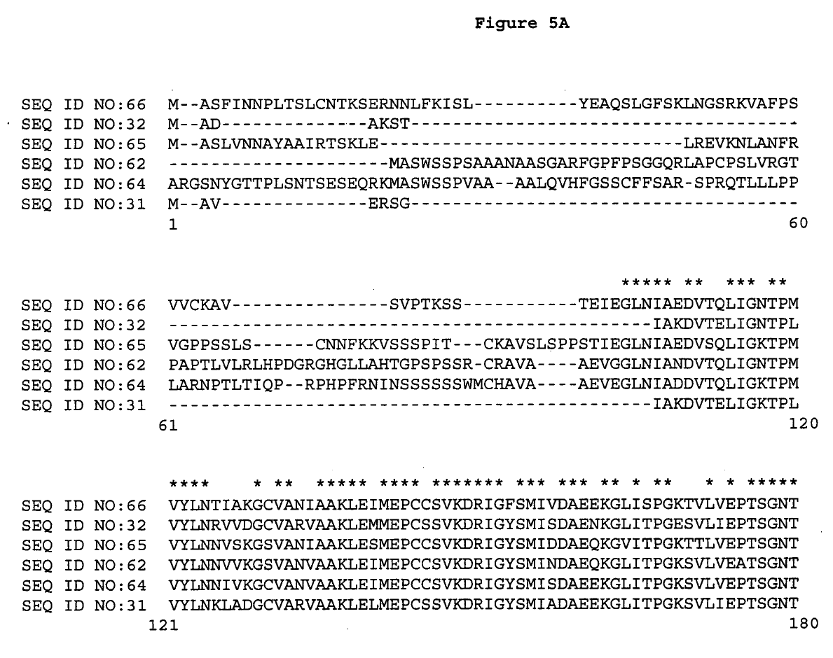

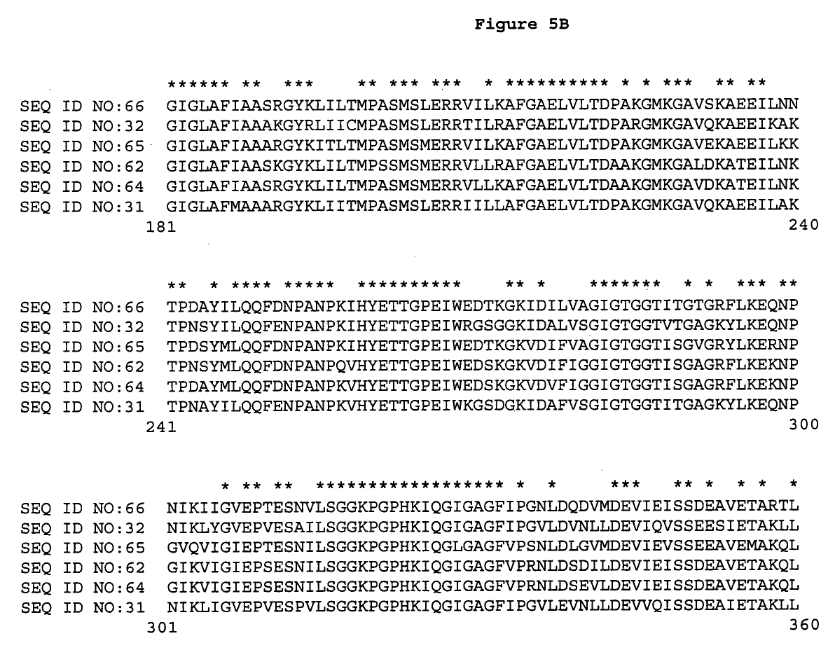

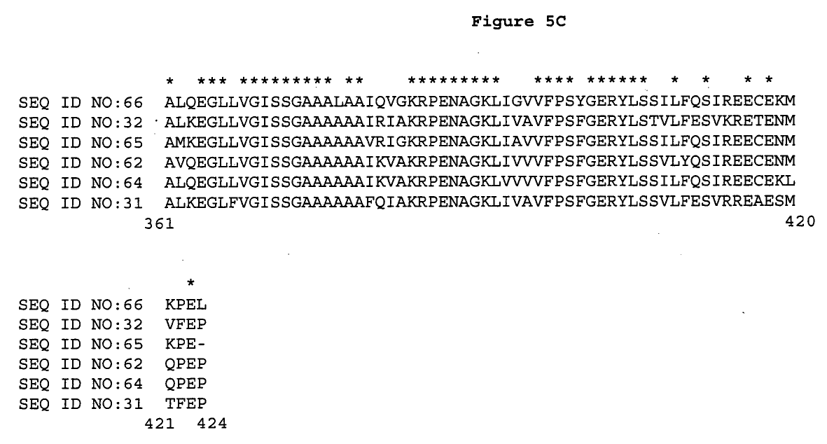

FIGS. 5A, 5B and 5C present an alignment of the amino acid sequences set forth in SEQ ID NOs:31, 62, and 64 with the Citrullus lanatus sequence (NCBI General Identifier No. 540497; SEQ ID NO:32), Spinacia oleracea (NCBI General Identifier No. 416869; SEQ ID NO:65), and the Solanum tuberosum sequence (NCBI General Identifier No. 11131628; SEQ ID NO:66). The data in Table 14 presents a calculation of the percent identity of the amino acid sequences set forth in SEQ ID NOs:31, 62, and 64 with the Citrullus lanatus sequence (NCBI General Identifier No. 540497; SEQ ID NO:32), Spinacia oleracea (NCBI General Identifier No. 416869; SEQ ID NO:65), and the Solanum tuberosum sequence (NCBI General Identifier No. 11131628; SEQ ID NO:66).

TABLE 14
Percent Identity of Amino Acid Sequences Deduced
From the Nucleotide Sequences of cDNA Clones
Encoding Polypeptides Homologous to Cysteine Synthase

| | | Percent Identity to | | |
|---|---|---|---|---|
| | Amino Acid | NCBI | NCBI | NCBI |
| Clone | SEQ ID NO. | GI 540497 | GI 416869 | GI 11131628 |
| se3.05h06 | 31 | 87.1 | 72.3 | 76.9 |
| Contig of: | 62 | 73.8 | 71.3 | 69.7 |
| cco1n.pk083.j4 | | | | |
| chp2.pk0016.b1 | | | | |
| cpd1c.pk004.b20 | | | | |
| cr1n.pk0083.c5 | | | | |
| csi1.pk0003.g6 | | | | |
| p0126.cnlcb49r | | | | |
| rls6.pk0068.b7:fis | 64 | 73.2 | 72.6 | 72.8 |

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode entire corn, rice, and soybean cysteine synthases. These sequences represent the first corn, rice, and soybean sequences encoding cysteine synthase known to Applicant.

### Example 7

### Characterization of cDNA Clones Encoding Cystathione β-Lyase

The BLASTX search using the EST sequences from clones listed in Table 15 revealed similarity of the polypeptides encoded by the cDNAs to cystathionine β-lyase from Arabidopsis thaliana (GenBank Accession No. L40511; NCBI General Identifier No. 1708993). Shown in Table 15 are the BLAST results for individual ESTs (“EST”), the sequences of the entire cDNA inserts comprising the indicated cDNA clones (“FIS”), or the sequences of FISs encoding the entire protein (“CGS”):

TABLE 15
BLAST Results for Sequences Encoding Polypeptides
Homologous to Cystathione β-Lyase

| | | BLAST pLog Score |
|---|---|---|
| Clone | Status | 1708993 (A. thaliana) |
| cen1.pk0061.d4 | FIS | 50.41 |
| rlr12.pk0026.g1 | EST | 39.00 |
| sfl1.pk0012.c4 | CGS | 33.85 |
| wr1.pk0091.g6 | EST | 52.52 |

The sequence of the entire cDNA insert in the clone wr1.pk0091.g6 was determined, RACE PCR was used to obtain the 5′ portion of the rice cystathionine β-lyase, and further sequencing and searching of the DuPont proprietary database allowed the identification of other corn and wheat clones encoding cystathionine β-lyase. The BLASTX search using the EST sequences from clones listed in Table 16 revealed similarity of the polypeptides encoded by the cDNAs to cystathionine β-lyase from Arabidopsis thaliana (GenBank Accession No. L40511; NCBI General Identifier No. 1708993). Shown in Table 16 are the BLAST results for the sequences of the entire cDNA inserts comprising the indicated cDNA clones (“FIS”), or the sequences encoding the entire protein derived from contigs assembled from the sequences of more than two ESTs, the sequence of contigs assembled from the entire cDNA inserts comprising the indicated cDNA clones and 5′ RACE PCR or an EST (“Contig*”):

TABLE 16
BLAST Results for Sequences Encoding Polypeptides
Homologous to Cystathione β-Lyase

| | | BLAST pLog Score |
|---|---|---|
| Clone | Status | 1708993 |
| Contig of: | Contig* | >180.00 |
| cen1.pk0061.d4 | | |
| p0005.cbmei71r | | |
| p0014.ctuui39r | | |
| p0109.cdadg47r | | |
| p0125.czaay16r | | |
| 5′ RACE PCR+ | Contig* | 178.00 |
| rlr12.pk0026.g1:fis | | |
| wr1.pk0091.g6:fis | FIS | 177.00 |

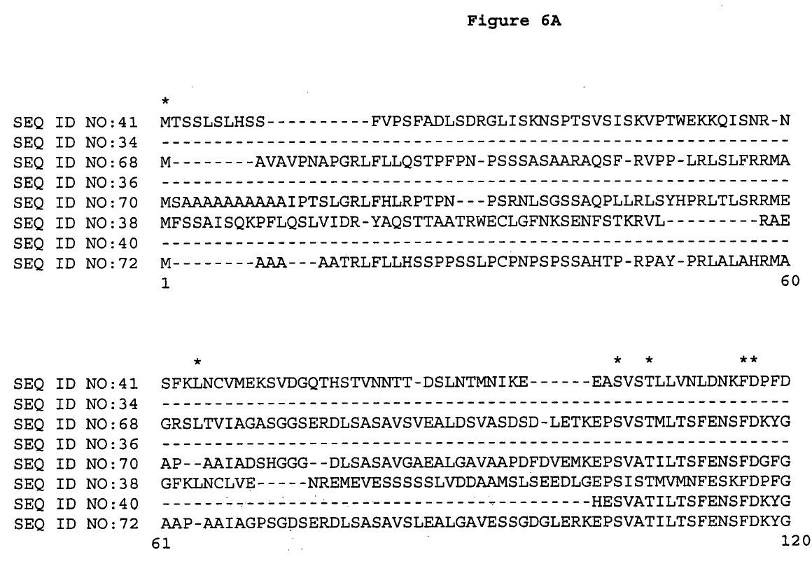

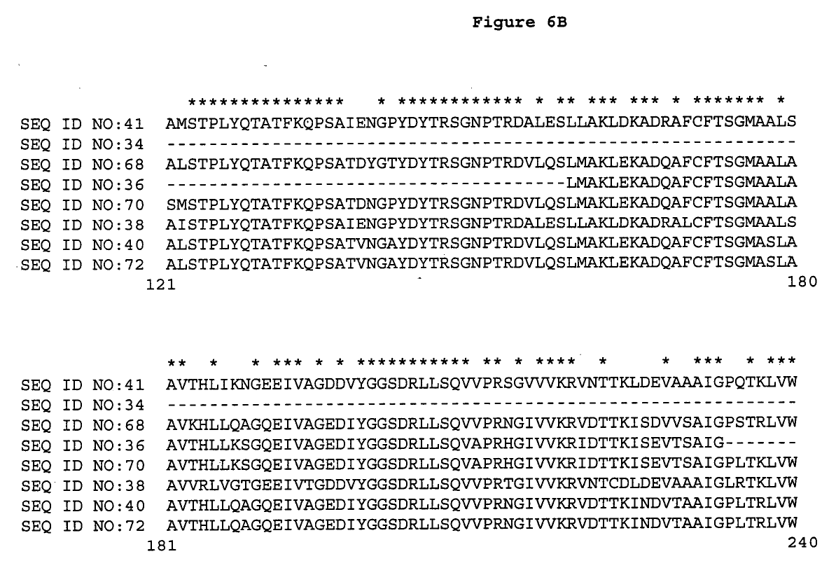

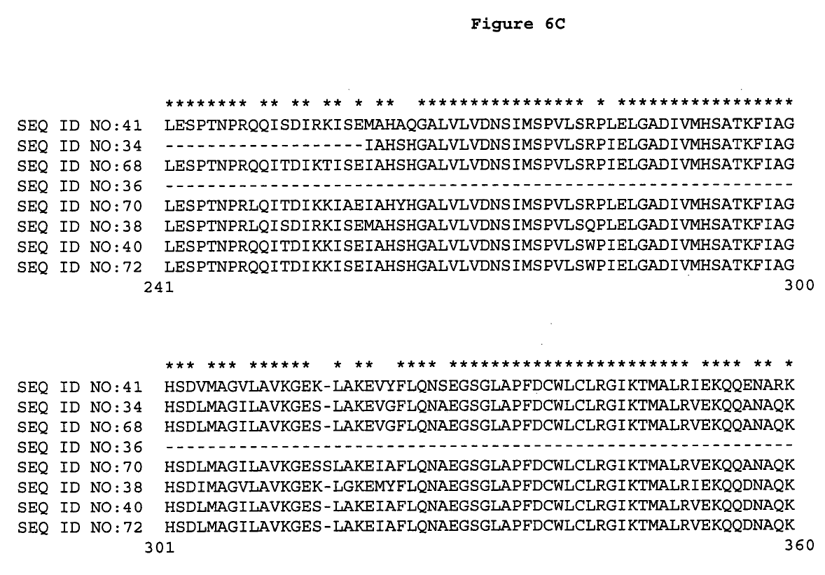

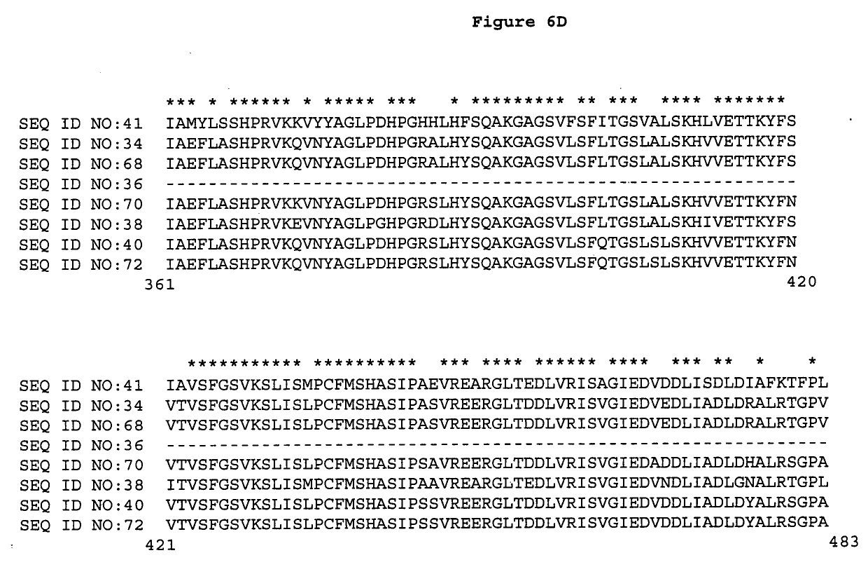

FIGS. 6A, 6B, 6C and 6D present an alignment of the amino acid sequences set forth in SEQ ID NOs:34, 36, 38, 40, 68, 70, and 72 with the Arabidopsis thaliana sequence (NCBI General Identifier No. 1708993; SEQ ID NO:41). The data in Table 17 presents a calculation of the percent identity of the amino acid sequences set forth in SEQ ID NOs:34, 36, 38, 40, 68, 70, and 72 with the Arabidopsis thaliana sequence (NCBI General Identifier No. 1708993; SEQ ID NO:41).

TABLE 17
Percent Identity of Amino Acid Sequences Deduced From
the Nucleotide Sequences of cDNA Clones Encoding
Polypeptides Homologous to Cystathione β-Lyase

| | | Percent Identity to |
|---|---|---|
| Clone | SEQ ID NO. | 1708993 (Arabidopsis thaliana) |
| cen1.pk0061.d4 | 34 | 83.0 |
| rlr12.pk0026.g1 | 36 | 76.0 |
| sfl1.pk0012.c4 | 38 | 72.2 |
| wr1.pk0091.g6 | 40 | 71.8 |
| Contig of: | 68 | 66.8 |
| cen1.pk0061.d4 | | |
| p0005.cbmei71r | | |
| p0014.ctuui39r | | |
| p0109.cdadg47r | | |
| p0125.czaay16r | | |
| 5′ RACE PCR+ | 70 | 66.2 |
| rlr12.pk0026.g1:fis | | |
| wr1.pk0091.g6:fis | 72 | 66.2 |

The amino acid sequence set forth in SEQ ID NO:34 is identical to amino acids 248 through 470 of the amino acid sequence set forth in SEQ ID NO:68. The amino acid sequence set forth in SEQ ID NO:36 is identical to amino acids 152 through 226 of the amino acid sequence set forth in SEQ ID NO:70. The amino acid sequence set forth in SEQ ID NO:40 is identical to amino acids 3 through 133 of the amino acid sequence set forth in SEQ ID NO:72.

Sequence alignments and percent identity calculations were performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode an entire soybean cystathionine β-lyase, a substantial portion and an entire corn and rice cystathionine β-lyases, a portion and a substantial portion of a wheat cystathionine β-lyase.

### Example 8

### Expression of Chimeric Genes in Monocot Cells

A chimeric gene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (Nco I or Sma I) can be incorporated into the oligonucleotides to provide proper orientation of the DNA fragment when inserted into the digested vector pML103 as described below. Amplification is then performed in a standard PCR. The amplified DNA is then digested with restriction enzymes Nco I and Sma I and fractionated on an agarose gel. The appropriate band can be isolated from the gel and combined with a 4.9 kb Nco 1-Sma I fragment of the plasmid pML103. Plasmid pML103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb Sal I-Nco I promoter fragment of the maize 27 kD zein gene and a 0.96 kb Sma I-Sal I fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue□; Stratagene). Bacterial transformants can be screened by restriction enzyme digestion of plasmid DNA and limited nucleotide sequence analysis using the dideoxy chain termination method (Sequenase□ DNA Sequencing Kit; U.S. Biochemical). The resulting plasmid construct would comprise a chimeric gene encoding, in the 5′ to 3′ direction, the maize 27 kD zein promoter, a cDNA fragment encoding the instant polypeptides, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### Example 9

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 10

### Expression of Chimeric Genes in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% low melting agarose gel. Buffer and agarose contain 10 μg/ml ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs (NEB), Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 □g/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25° C. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 11

### Evaluating Compounds for Their Ability to Inhibit the Activity of Plant Biosynthetic Enzymes

The polypeptides described herein may be produced using any number of methods known to those skilled in the art. Such methods include, but are not limited to, expression in bacteria as described in Example 10, or expression in eukaryotic cell culture, in planta, and using viral expression systems in suitably infected organisms or cell lines. The instant polypeptides may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the fusion protein and the enzyme.

Purification of the instant polypeptides, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the instant polypeptides are expressed as fusion proteins, the purification protocol may include the use of an affinity resin which is specific for the fusion protein tag attached to the expressed enzyme or an affinity resin containing ligands which are specific for the enzyme. For example, the instant polypeptides may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include β-mercaptoethanol or other reduced thiol. The eluted fusion protein may be subjected to further purification by traditional means as stated above, if desired. Proteolytic cleavage of the thioredoxin fusion protein and the enzyme may be accomplished after the fusion protein is purified or while the protein is still bound to the ThioBond™ affinity resin or other resin.

Crude, partially purified or purified enzyme, either alone or as a fusion protein, may be utilized in assays for the evaluation of compounds for their ability to inhibit enzymatic activation of the instant polypeptides disclosed herein. Assays may be conducted under well known experimental conditions which permit optimal enzymatic activity. Examples of assays for many of these enzymes can be found in Methods in Enzymology Vol. V, (Colowick and Kaplan eds.) Academic Press, New York or Methods in Enzymology Vol. XVII, (Tabor and Tabor eds.) Academic Press, New York. Specific examples may be found in the following references, each of which is incorporated herein by reference: aspartic semialdehyde dehydrogenase may be assayed as described in Black et al. (1955) J. Biol. Chem. 213:39-50, or Cremer et al. (1988) J. Gen. Microbiol. 134:3221-3229; diaminopimelate decarboxylase may be assayed as described in Work (1962) in Methods in Enzymology Vol. V, (Colowick and Kaplan eds.) 864-870, Academic Press, New York or Cremer et al. (1988) J. Gen. Microbiol. 134:3221-3229; homoserine kinase may be assayed as described in Aarnes (1976) Plant Sci. Lett. 7:187-194; cysteine synthase may be assayed as described in Thompson et al. (1968) Biochem. Biophys. Res. Commun. 31: 281-286 or Bertagnolli et al. (1977) Plant Physiol. 60:115-121; and cystathionine β-lyase may be assayed as described in Giovanelli et al. (1971) Biochim. Biophys. Acta 227:654-670 or Droux et al. (1995) Arch. Biochem Biophys. 316:585-595.

### Example 12

### Characterization of cDNA Clones Encoding Cysteine Synthase

Characterization of cDNAs encoding soybean cysteine synthase follow. The BLASTX search using the EST sequences from clones listed in Table 17 revealed similarity of the polypeptides encoded by the cDNAs to cysteine synthase from Citrullus lanatus (NCBI General Identifier No. 540497; SEQ ID NO:32), O-acetylserine (thiol)lyase from Populus alba x Populus tremula (NCBI General Identifier No. 34099833; SEQ ID NO:89), plastidic cysteine synthase 1 from Solanum tuberosum (NCBI General Identifier No. 12081919; SEQ ID NO:90), SEQ ID NO:198006 in U.S. 20004031072 (SEQ ID NO:91) and SEQ ID NO:129998 in U.S. 20004123343 (SEQ ID NO:92). Shown in Table 17 are the BLASTP results obtained for the amino acid sequences of the cysteine synthases encoded by the entire cDNA inserts comprising the indicated cDNA clones.

TABLE 17
BLAST Results for Sequences Encoding Polypeptides
Homologous to Cysteine Synthase

| | NCBI General | |
|---|---|---|
| | Identifier No. | |
| | or | |
| Clone | SEQ ID NO in US Patent | |
| (SEQ ID NO:) | Application No. | BLAST pLog Score |
| src3c.pk002.e24:fis | NCBI GI 540497 | 164 |
| (SEQ ID NO: 74) | (SEQ ID NO: 32) | |
| src3c.pk002.e24:fis | SEQ ID NO: 198006 in | 179 |
| (SEQ ID NO: 74) | US 2004031072 | |
| | (SEQ ID NO: 91) | |
| Contig of: | NCBI GI 540497 | 164 |
| sl1.pk0067.g6 (5′ SID) | (SEQ ID NO: 32) | |
| sl1.pk0076.b3:fis | | |
| (SEQ ID NO: 76) | | |
| Contig of: | SEQ ID NO: 198006 in | 176 |
| sl1.pk0067.g6 (5′ SID) | US 2004031072 | |
| sl1.pk0076.b3:fis | (SEQ ID NO: 91) | |
| (SEQ ID NO: 76) | | |
| Contig of: | NCBI GI 12081919 | 163 |
| sdr1f.pk004.d11 (5′ | (SEQ ID NO: 90) | |
| SID) | | |
| sic1c.pk002.c7 | | |
| sr1.pk0043.d9 | | |
| sl2.pk0005.c5 | | |
| sdp2c.pk012.d22 | | |
| sdp2c.pk014.d22 | | |
| sdr1f.pk004.d11.f | | |
| (SEQ ID NO: 78) | | |
| Contig of: | SEQ ID NO: 129998 in | 165 |
| sdr1f.pk004.d11 (5′ | US 20040123343 | |
| SID) | (SEQ ID NO: 92) | |
| sic1c.pk002.c7 | | |
| sr1.pk0043.d9 | | |
| sl2.pk0005.c5 | | |
| sdp2c.pk012.d22 | | |
| sdp2c.pk014.d22 | | |
| sdr1f.pk004.d11.f | | |
| (SEQ ID NO: 78) | | |

The nucleotide sequence corresponding to the entire cDNA insert in clone src3c.pk002.e24:fis is shown in SEQ ID NO:73; the amino acid sequence corresponding to the translation of nucleotides 132 through 1106 is shown in SEQ ID NO:74 (nucleotides 1107-1109 encode a stop). The nucleotide sequence corresponding to contig of contig of: sl1.pk0067.g6 (5′ SID) and sl1.pk0076.b3:fis is shown in SEQ ID NO:75; the amino acid sequence corresponding to the translation of nucleotides 142 through 1116 is shown in SEQ ID NO:76 (nucleotides 1117-1119 encode a stop). The nucleotide sequence corresponding to the contig of: sdr1f.pk004.d11 (5′ SID), sic1c.pk002.c7, sr1.pk0043.d9, sl2.pk0005.c5, sdp2c.pk012.d22, sdp2c.pk014.d22 and sdr1f.pk004.d11.f is shown in SEQ ID NO:77; the amino acid sequence corresponding to the translation of nucleotides 85 through 1266 is shown in SEQ ID NO:78 (nucleotides 1267-1269 encode a stop).

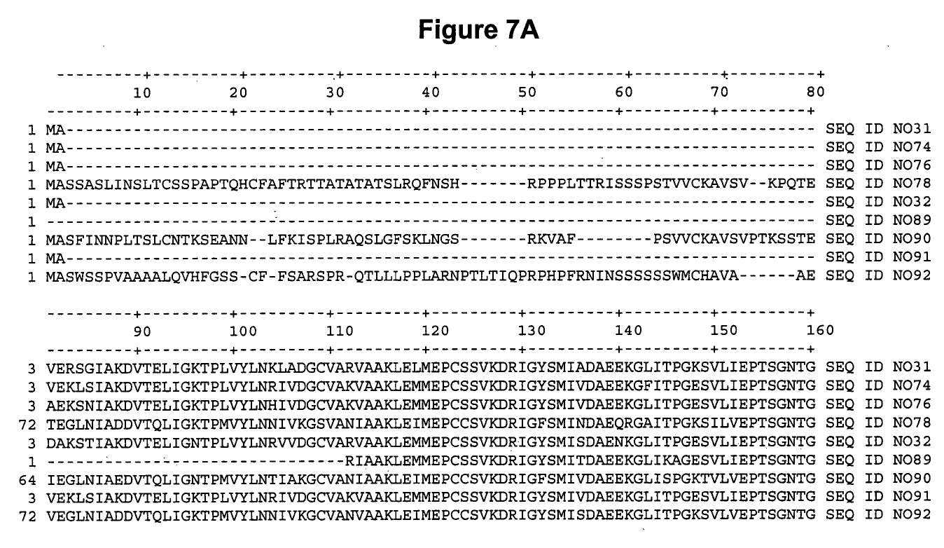

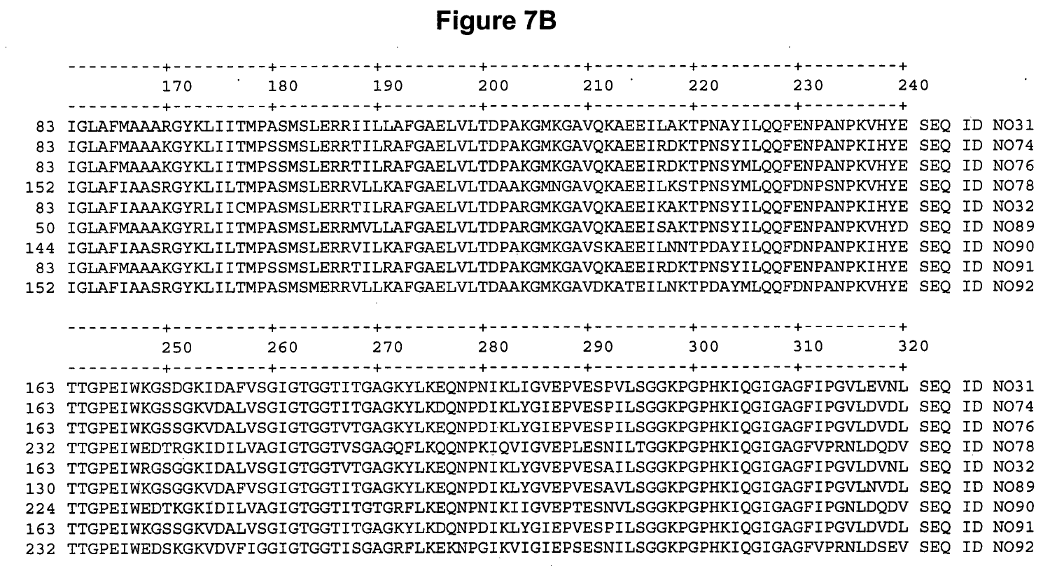

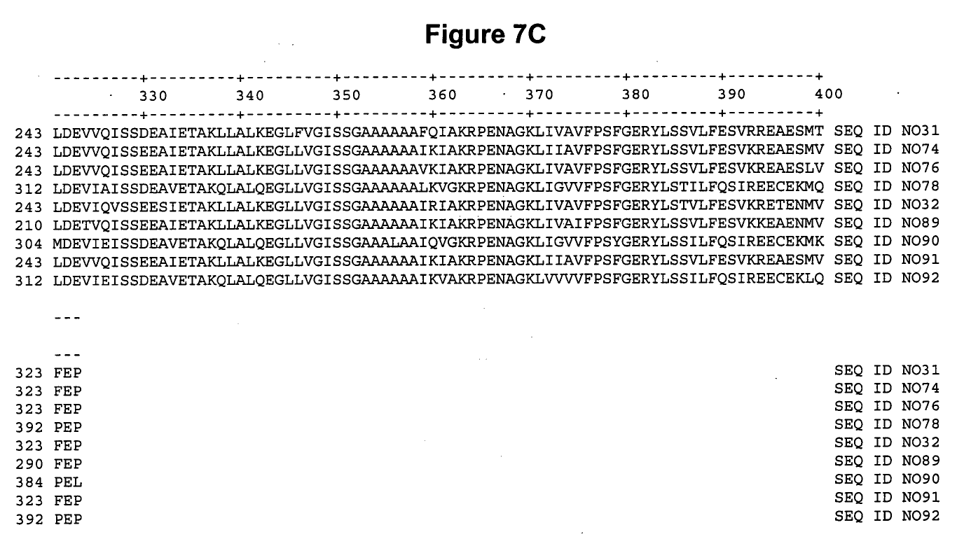

FIGS. 7A, 7B and 7C present an alignment of the amino acid sequences set forth in SEQ ID NOs:31, 74, 76 and 78, the amino acid sequence from Citrullus lanatus (NCBI General Identifier No. 540497; SEQ ID NO:32), O-acetylserine (thiol)lyase from Populus alba x Populus tremula (NCBI General Identifier No. 34099833; SEQ ID NO:89), plastidic cysteine synthase 1 from Solanum tuberosum (NCBI General Identifier No. 12081919; SEQ ID NO:90), SEQ ID NO:198006 in U.S. 20004031072 (SEQ ID NO:91) and SEQ ID NO:129998 in U.S. 20004123343 (SEQ ID NO:92).

The data in Table 18 presents the results obtained for the calculation of the percent identity of the amino acid sequences set forth in SEQ ID Nos:74, 76 and 78, with the cysteine synthase sequences from either Citrullus lanatus (NCBI General Identifier No. 540497; SEQ ID NO:32), O-acetylserine (thiol)lyase from Populus alba x Populus tremula (NCBI General Identifier No. 34099833; SEQ ID NO:89), plastidic cysteine synthase 1 from Solanum tuberosum (NCBI General Identifier No. 12081919; SEQ ID NO:90), SEQ ID NO:198006 in U.S. 20004031072 (SEQ ID NO:91) or SEQ ID NO:129998 in U.S. 20004123343 (SEQ ID NO:92).

TABLE 18
Percent Identity of Deduced Amino Acid Sequences of the
cDNA clones Encoding Polypeptides
Homologous to Cysteine Synthase

| | | Percent Identity |
|---|---|---|
| | | to NCBI GI # or |
| Clone | SEQ | Percent Identity to SEQ ID NO in US |
| (SEQ ID NO:) | ID NO. | Patent Application No. |
| src3c.pk002.e24:fis | 74 | 89% to NCBI GI 540497 |
| (SEQ ID NO: 74) | | (SEQ ID NO: 32) |
| src3c.pk002.e24:fis | 74 | 99% to SEQ ID NO: 198006 in |
| (SEQ ID NO: 74) | | US 2004031072 |
| | | (SEQ ID NO: 91) |
| Contig of: | 76 | 89% to NCBI GI 540497 |
| sl1.pk0067.g6 (5′ SID) | | (SEQ ID NO: 32) |
| sl1.pk0076.b3:fis | | |
| (SEQ ID NO: 76) | | |
| Contig of: | 76 | 96% SEQ ID NO: 198006 in |
| sl1.pk0067.g6 (5′ SID) | | US 2004031072 |
| sl1.pk0076.b3:fis | | (SEQ ID NO: 91) |
| (SEQ ID NO: 76) | | |
| Contig of: | 78 | 84% to NCBI GI 12081919 |
| sdr1f.pk004.d11 (5′ SID) | | (SEQ ID NO: 90) |
| sic1c.pk002.c7 | | |
| sr1.pk0043.d9 | | |
| sl2.pk0005.c5 | | |
| sdp2c.pk012.d22 | | |
| sdp2c.pk014.d22 | | |
| sdr1f.pk004.d11.f | | |
| (SEQ ID NO: 78) | | |
| Contig of: | 78 | 86% SEQ ID NO: 129998 in |
| sdr1f.pk004.d11 (5′ SID) | | US 20040123343 |
| sic1C.pk002.c7 | | (SEQ ID NO: 92) |
| sr1.pk0043.d9 | | |
| sl2.pk0005.c5 | | |
| sdp2c.pk012.d22 | | |
| sdp2c.pk014.d22 | | |
| sdr1f.pk004.d11.f | | |
| (SEQ ID NO: 78) | | |

Sequence alignments and percent identity calculations were performed using the Megalign program of the LASARGENE bioinformatics computing suite (DNASTAR Inc., Madison, Wis.). Multiple alignment of the sequences was performed using the Clustal method of alignment (Higgins and Sharp, CABIOS. 5:151-153 (1989)) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode cysteine synthases.

### Example 13

### Expression Analysis of Soybean Cysteine Synthase Homologs

The expression pattern of soybean cysteine synthase (also known as O-acetylserine (thiol) lyase—OASTL) was analyzed.

The expression study was conducted by comparing MPSS (Massively Parallel Signature Sequencing) data (Brenner et al., Nature Biotechnology 18:630-634 (2000); Brenner et al., Proc Natl. Acad. Sci USA 97:1665-1670 (2000)), obtained from various soybean tissues of different lines. MPSS data enabled a survey of expression levels in terms of looking at the abundance of particular cDNA clones among 1,000,000 clones for each library. The relative abundance of a particular tagged sequence, which is unique to a single cDNA, correlates with the relative level of accumulation of the corresponding RNA in that tissue.

Cysteine is a key metabolic compound with several cellular functions being a proteinogenic amino acid, sulfur donor, or part of protective metabolites (Hesse et al., J. Exp. Bot. 55:1283-1292 (2004)). Cysteine synthesis can be regulated at multiple steps in which the serine acetyltransferase/O-acetylserine (thiol) lyase (SAT/OASTL) complex is identified as one of the key regulators. There are at least four different OASTL genes in soybean genome. Based on the sequence information, Applicants predict that three of them belong to cytosolic isoform (SEQ ID Nos:31, 74 and 76) and the fourth is chloroplast isoform (SEQ ID NO:78). The results of the gene expression profiling data indicated that different OASTL genes have different expression patterns in terms of tissue specificity. The tissue-specific expression patterns of the three OASTL genes from the cytosolic isoform family are shown below in Table 19. Also shown in Table 19 are the tissue-specific expression patterns of two SAT genes (previously disclosed in U.S. Pat. No. 6,548,280, issued Apr. 15, 2003, and is herein incorporated by reference). (The number designations (i.e., 1, 2 and 3) are provided for clarity of the instant specification.) The OASTL1 (se3.05h06; SEQ ID NO:30 is the entire nucleotide sequence) gene is more expressed in leaf and seeds as compared to root. The OASTL2 (src3c.pk002.e24:fis; SEQ ID NO:73 is the entire nucleotide sequence) gene is more expressed in roots and the OASTL3 (contig of: sl1.pk0067.g6 (5′ SID) nad sl1.pk0076.b3:fis; SEQ ID NO:75 is the entire nucleotide sequence) is more seed specific.

Applicants believe that the relative OASTL expression level and enzyme activities in different soybean tissues play critical roles in regulation of the cysteine biosynthetic pathway and sulfur assimilation pathway. More importantly, the proper balance and the ratio between SAT and OASTL enzyme activities in a specific tissue are also very important for cysteine biosynthesis. By either reducing or increasing expression of certain OASTL genes in a constitutive, seed-specific or root-specific manner, it is believed that one can modulate the ratios between SAT and OASTL expression level and enzyme activities to regulate the sulfur assimilation and cysteine biosynthetic pathway.

For example, the native expression of the OASTL2 gene is very high in roots compared to its expression in leaf and seeds. The root-specific cosuppression of the OASTL2 gene will reduce the OASTL2 expression and the enzyme activities in the root so its modified tissue-specific expression pattern is more comparable to the native SAT1 and SAT2 gene expression. On the other hand, the native expression pattern of the OASTL1 is already comparable to the expression of native SATs. Constitutive overexpression of the OSATL1 gene will increase its expression level while maintaining its native tissue specificity. Finally, simultaneous overexpression of both the SAT2 and an OASTL1 constitutively is another way to modulate cysteine biosynthesis while maintaining its native tissue specificity and SAT/OASTL ratios. The following six examples illustrate overexpression or cosuppression of two specific OASTL genes described in the instant specification. However, the strategies described here can be applied to any other OASTL genes to achieve the same goals.

TABLE 19
Expression Analysis

| | Leaf | Root | Seed |
|---|---|---|---|
| | Expression | Expression | Expression |
| Clone | (in PPM) | (in PPM) | (in PPM) |
| se3.05h06 | 101 | 44 | 174 |
| OASTL1 | | | |
| (SEQ ID NO: 31) | | | |
| src3c.pk002.e24:fis | 209 | 383 | 214 |
| OASTL2 | | | |
| (SEQ ID NO: 74) | | | |
| Contig of: | | | |
| sl1.pk0067.g6 (5′ | 0 | 13 | 186 |
| SID) | | | |
| sl1.pk0076.b3:fis | | | |
| OASTL3 | | | |
| (SEQ ID NO: 76) | | | |
| sr1.pk0162.a9 | 17 | 4 | 23 |
| SAT1 | | | |
| (SEQ ID NO: 93) | | | |
| srm.pk0021.f11 | 191 | 59 | 124 |
| SAT2 | | | |
| (SEQ ID NO: 93) | | | |

### Example 14

### Preparation of Recombinant Constructs for Seed-Specific Overexpression of OASTL in Soybean

A soybean expression vector with a seed-specific promoter (ZBL119 GY1) (examples of seed-specific promoters include, but are not limited to, the promoters of seed storage proteins) is made from soybean expression vector ZBL119 (plasmid ZBL119 has been previously described in Example 2 of WO2004/071467, published Aug. 26, 2004, and is herein incorporated by reference). The ZBL119 plasmid is digested with Not I. The 7121 bp fragment containing T7 promoter:hygromycin:T7 terminator, 35S promoter:hygromycin:NOS terminator, GY1 promoter:phaseolin terminator is separated from the 1382 bp M. alpina delta-6 fragment by gel electrophoresis and self-ligated to make the ZBL119 GY1. The open reading frame of the soybean cysteine synthase (also known as O-acetylserine (thiol) lyase—OASTL) gene is PCR-amplified from the soybean EST clone (se3.05h06; SEQ ID NO:30) using a primer 1 (SEQ ID:79) and a primer 2 (SEQ ID:80), which is designed to introduce a Not I restriction sites at both ends of the soybean OASTL coding sequence.

| | |
|---|---|
| GAATTCGCGGCCGCATGGCTGTTGAAAGGTCCGGA | (SEQ ID NO:79) |
| ATTGC | |
| GAATTCGCGGCCGCTCAGGGCTCAAAAGTCATGCT | (SEQ ID NO:80) |
| TTCAG | |

The resulting PCR fragment is subcloned into the intermediate cloning vector pGEM-T (Promega) according the manufacturer's protocol. The OASTL coding sequence is then released by Not I digestion and cloned into the Not I site of the soybean expression vector ZBL119 GY1, described above, to obtain the construct ZBL119 GY1 OASTL. In this construct, the OASTL1 coding sequence (SEQ ID NO:81) is driven by a soybean seed specific Glycinin 1 promoter to achieve seed-specific overexpression.

### Example 15

### Preparation of Recombinant Constructs for Constitutive Overexpression of OASTL in Soybean

A soybean expression vector with a constitutive promoter (ZBL119 SCP1) is made from the soybean expression vector ZBL119 GY1 described in Example 14. The ZBL119 GY1 vector is digested with BamH I and Not I. The 6426 bp fragment containing T7 promoter:hygromycin:T7 terminator, 35S promoter:hygromycin:NOS terminator, phaseolin terminator is separated from the 691 bp GY1 promoter fragment by gel electrophoresis. A PCR fragment containing the SCP1 promoter is obtained using a primer 3 (SEQ ID NO:82) and a primer 4 (SEQ ID NO:83) with a plasmid PHP9778 (plasmid PHP9778 has been previously described in U.S. Pat. No. 6,555,673 B1, issued Apr. 29, 2003, and is herein incorporated by reference) as a template.

| | |
|---|---|
| GAATTCGGATCCGATCCGTCAACATGGTGGAGCAC | (SEQ ID NO:82) |
| GAATTCGCGGCCGCTGTAATTGTAAATAGTAATTG | (SEQ ID NO:83) |

The 556 bp PCR fragment of the synthetic SCP1 promoter is then digested with BamH I and Not I and ligated to the 6426 bp ZBL119 GY1 vector cut by BamH I and Not I. The vector formed is ZBL119 SCP1. The soybean OASTL1 coding sequence in the GEM-T vector described in Example 14 is then released by Not I digestion and cloned into the Not I site of the soybean expression vector ZBL119 SCP1 to obtain the construct ZBL119 SCP1 OASTL1. In this construct, the OASTL1 coding sequence (SEQ ID NO:81) is driven by a synthetic constitutive SCP1 promoter to achieve constitutive overexpression.

### Example 16

### Preparation of Recombinant Constructs for Root-Specific Overexpression of OASTL in Soybean

A soybean expression vector with a root-preferred promoter (ZBL119 IFS1) is made from the soybean expression vector ZBL119 GY1 as describe in Example 14. The ZBL119 GY1 vector is digested with BamH I and Not I. The 6426 bp fragment containing T7 promoter:hygromycin:T7 terminator, 35S promoter:hygromycin:NOS terminator, phaseolin terminator is separated from the 691 bp GY1 promoter fragment by gel electrophoresis. A PCR fragment containing the soybean IFS1 promoter (Subramanian et al., Plant Mol. Biol. 54:623-639 (2004)) is obtained using a primer 5 (SEQ ID NO:84) and a primer 6 (SEQ ID NO:85) with soybean genomic DNA as template.

| | |
|---|---|
| GAATTCGGATCCACTACGCTTTGAAGGAGCACGTG | (SEQ ID NO:84) |
| GAATTCGCGGCCGCCGTGAAACCTCAGTGCAAGAA | (SEQ ID NO:85) |

The 2545 bp PCR fragment of the soybean IFS1 promoter is then digested with BamH I and Not I and ligated to the 6426 bp ZBL119 GY1 vector cut by BamH I and Not I. The vector formed is ZBL119 IFS1. The soybean OASTL1 coding sequence in the pGEM-T vector described in Example 14 is then released by Not I digestion and cloned into the Not I site of the soybean expression vector ZBL119 IFS1 to obtain the construct ZBL119 IFS1 OASTL1. In this construct, the OASTL1 coding sequence (SEQ ID NO:81) is driven by a root-preferred soybean IFS1 promoter to achieve root-preferred overexpression.

### Example 17

### Preparation of Recombinant Constructs for Root-Specific OASTL Cosuppression in Soybean

Sequence analyses of soybean OASTL genes indicate that the 5′ ORF regions are more divergent than the 3′ ORF region (see FIGS. 7A, 7B and 7C). In order to achieve gene-specific cosuppression without interfering other OASTL genes, a fragment of the soybean OASTL2 gene was PCR amplified from the soybean EST clones (src3c.pk002.e24; SEQ ID NO:73) using a primer 7 (SEQ ID NO:86) and a primer 8 (SEQ ID NO:87), which was designed to introduce Not I restriction sites at both ends of the soybean OASTL2 gene.

| | |
|---|---|
| GAATTCGCGGCCGCGCGGTTGAGAAGTTGAGCATT | (SEQ ID NO:86) |
| GCAAA | |
| GAATTCGCGGCCGCTGCCAAACCTATGCCCGTGTT | (SEQ ID NO:87) |
| TCCA | |

The resulting PCR fragment is subcloned into the intermediate cloning vector PGEM-T (Promega) according the manufacturer's protocol. The OASTL2 fragment is then released by Not I digestion and cloned into the Not I site of the soybean seed-specific expression vector ZBL119 GY1 described in Example 14 to obtain the construct ZBL119 GY1 OASTL2-TR. In this construct, the OASTL2 fragment (SEQ ID NO:88) is driven by a soybean seed-specific Glycinin 1 promoter to achieve seed-specific cosuppression

### Example 18

### Preparation of Recombinant Constructs for Constitutive OASTL Cosuppression in Soybean

The same OASTL2 PCR fragment (SEQ ID NO:87) in the pGEM-T vector as described in the Example 17 is cloned into the Not I site of the soybean constitutive-expression vector ZBL119 SCP1 described in the Example 15 to obtain the construct ZBL119 SCP1 OASTL2-TR. In this construct, the OASTL2 fragment (SEQ ID NO:88) is driven by a constitutive SCP1 promoter to achieve constitutive cosuppression.

### Example 19

### Preparation of Recombinant Constructs for Root-Specific OASTL Cosuppression in Soybean

The same OASTL2 PCR fragment (SEQ ID NO:87) in the PGEM-T vector as described in the Example 17 is cloned into the Not I site of the soybean root-preferred expression vector ZBL119 IFS1 described in the Example 16 to obtain the construct ZBL119 IFS1 OASTL2-TR. In this construct, the OASTL2 fragment (SEQ ID NO:88) is driven by a soybean root-preferred IFS1 promoter to achieve root-preferred cosuppression.

### Example 20

### Transformation of Somatic Soybean Embryo Cultures and Regeneration of Soybean Plants

The following example sets forth a protocol which can be used for transformation of soybean via particle bombardment of embryogenic tissue. Those skilled in the art will appreciate that a number of minor variations can be made to the protocol described below. Such transformed somatic embryos are also suitable for germination. The following protocol is also set forth in PCT Publication No. WO 02/00904, which published on Jan. 3, 2002, and is herein incorporated by reference.

Generic Stable Soybean Transformation Protocol:

Soybean embryogenic suspension cultures are maintained in 35 mL liquid media (SB55 or SBP6; see Table 20) on a rotary shaker, 150 rpm, at 28° C. with mixed fluorescent and incandescent lights on a 16:8 hour day/night schedule. Cultures are subcultured every four weeks by inoculating approximately 35 mg of tissue into 35 mL of liquid medium.

TABLE 20
Stock Solutions (g/L):
MS Sulfate 100X Stock

| | |
|---|---|
| MgSO₄.7H₂O | 37.0 |
| MnSO₄.H₂O | 1.69 |
| ZnSO₄.7H₂O | 0.86 |
| CuSO₄.5H₂O | 0.0025 |

MS Halides 100X Stock

| | |
|---|---|
| CaCl₂.2H₂O | 44.0 |
| Kl | 0.083 |
| CoCl₂.6H₂0 | 0.00125 |
| KH₂PO₄ | 17.0 |
| H₃BO₃ | 0.62 |
| Na₂MoO₄.2H₂O | 0.025 |

MS FeEDTA 100X Stock

| | |
|---|---|
| Na₂EDTA | 3.724 |
| FeSO₄.7H₂O | 2.784 |

B5 Vitamin Stock

10 g m-inositol
100 mg nicotinic acid
100 mg pyridoxine.HCl
1 g thiamine

SB55 (per Liter, pH 5.7)

10 mL each MS stocks
1 mL B5 vitamin stock
0.8 g NH₄NO₃
3.033 g KNO₃
1 mL 2,4-D (10 mg/mL stock)
60 g sucrose
0.667 g asparagine

SBP6

Same as SB55 except 0.5 mL 2,4-D

SB103 (per Liter, pH 5.7)

1X MS salts
6% maltose
750 mg MgCl₂
0.2% gelrite

SB71-1 (per Liter, pH 5.7)

1X B5 salts
1 mL B5 vitamin stock
3% sucrose
750 mg MgCl₂
0.2% gelrite

Soybean embryogenic suspension cultures are transformed with pTC3 by the method of particle gun bombardment (Klein et al., Nature 327:70 (1987)). A DuPont Biolistic PDS1000/HE instrument (helium retrofit) is used for these transformations.

To 50 mL of a 60 mg/mL 1 μm gold particle suspension is added (in order); 5 μL DNA (1 μg/μL), 20 μL spermidine (0.1 M), and 50 μL CaCl₂ (2.5 M). The particle preparation is agitated for 3 min, spun in a microfuge for 10 sec and the supernatant removed. The DNA-coated particles are then washed once in 400 μL 70% ethanol and resuspended in 40 μL of anhydrous ethanol. The DNA/particle suspension is sonicated three times for 1 sec each. Five μL of the DNA-coated gold particles are then loaded on each macro carrier disk. For selection, the plasmid contains a gene conferring resistance to hygromycin phosphotransferase (HYG) operably linked to an appropriate promoter. It is known by those skilled in the art that herbicide resistance can be used to select transformed plants in tissue culture, e.g., a mutated version of the acetolactate synthase (ALS) gene confers resistance to some sulfonylurea herbicides.

Approximately 300-400 mg of a four week old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1000 psi and the chamber is evacuated to a vacuum of 28 inches of mercury. The tissue is placed approximately 3.5 inches away from the retaining screen and bombarded three times. Following bombardment, the tissue is placed back into liquid and cultured as described above.

Eleven days post bombardment, the liquid media is exchanged with fresh SB55 containing 50 mg/mL hygromycin. The selective media is refreshed weekly. Seven weeks post bombardment, green, transformed tissue is observed growing from untransformed, necrotic embryogenic clusters. Isolated green tissue is removed and inoculated into individual flasks to generate new, clonally propagated, transformed embryogenic suspension cultures. Thus each new line is treated as an independent transformation event. These suspensions can then be maintained as suspensions of embryos maintained in an immature developmental stage or regenerated into whole plants by maturation and germination of individual somatic embryos.

Independent lines of transformed embryogenic clusters are removed from liquid culture and placed on a solid agar media (SB103) containing no hormones or antibiotics. Embryos are cultured for four weeks at 26° C. with mixed fluorescent and incandescent lights on a 16:8 hour day/night schedule.

It should be noted that any detectable phenotype, resulting from the co-suppression of a target nucleic acid fragment can be screened at this stage. The phenotype of transgenic soybean somatic embryos is predictive of seed phenotypes from resulting regenerated plants. This is further discussed in PCT Publication No. WO 02/00904, which published on Jan. 3, 2002. Detectable phenotypes include, but not be limited to, alterations in protein content, carbohydrate content, growth rate, viability, or the ability to develop normally into a soybean plant.

Furthermore, somatic embryos are also suitable for germination after eight weeks and can be removed from the maturation medium and dried in empty petri dishes for one to five days. The dried embryos can then be planted in SB71-1 medium where they will be allowed to germinate under the same lighting and germination conditions described above. Germinated embryos can be transferred to sterile soil and grown to maturity. Seeds can be harvested and analyzed for alteration in such things as their fatty acid compositions.

### Example 21

### Analysis of Amino Acid Content of the Seeds of Transformed Plants

To analyze for expression of the chimeric genes in seeds and for the consequences of expression on the amino acid content in the seeds, a seed meal can be prepared by any of a number of suitable methods known to those skilled in the art. The seed meal can be partially or completely defatted, via hexane extraction for example, if desired. Protein extracts can be prepared from the meal and analyzed for enzyme activity. Alternatively the presence of any of the expressed enzymes can be tested for immunologically by methods well-known to those skilled in the art. To measure free amino acid composition of the seeds, free amino acids can be extracted from the meal and analyzed by methods known to those skilled in the art (Bieleski et al., Anal. Biochem. 17:278-293 (1966)). Amino acid composition can then be determined using any commercially available amino acid analyzer. To measure total amino acid composition of the seeds, meal containing both protein-bound and free amino acids can be acid hydrolyzed to release the protein-bound amino acids and the composition can then be determined using any commercially available amino acid analyzer. Seeds expressing the instant amino acid biosynthetic enzymes and with altered lysine, threonine, methionine, cysteine and/or isoleucine content as compared to the wild type seeds can thus be identified and propagated.

To measure free amino acid composition of the seeds, free amino acids can be extracted from 8-10 milligrams of the seed meal in 1.0 mL of methanol/chloroform/water mixed in ratio of 12v/5v/3v (MCW) at room temperature. The mixture can be vortexed and then centrifuged in an eppendorf microcentrifuge for about 3 min; approximately 0.8 mL of supernatant is then decanted. To this supernatant, 0.2 mL of chloroform is added followed by 0.3 mL of water. The mixture is then vortexed and centrifuged in an eppendorf microcentrifuge for about 3 min. The upper aqueous phase, approximately 1.0 mL, can then be removed and dried down in a Savant Speed Vac Concentrator. The samples are then hydrolyzed in 6N hydrochloric acid, 0.4% β-mercaptoethanol under nitrogen for 24 h at 110-120° C. Ten percent of the sample can then be analyzed using a Beckman Model 6300 amino acid analyzer using post-column ninhydrin detection. Relative free amino acid levels in the seeds are then compared as ratios of lysine, threonine, methionine, cysteine and/or isoleucine to leucine, thus using leucine as an internal standard.

What is claimed is:

1. An isolated polynucleotide sequence comprising:
(a) a nucleotide sequence encoding a polypeptide having cysteine synthase activity, wherein the polypeptide has an amino acid sequence of at least 90% sequence identity, based on the Clustal method of alignment, when compared to one of SEQ ID NO:74, 76 or 78;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

2. The polynucleotide of claim 1 wherein the amino acid sequence of the polypeptide has at least 95% sequence identity, based on the Clustal method of alignment.

3. The polynucleotide of claim 1 wherein the nucleotide sequence comprises one of SEQ ID NO:73, 75 or 77.

4. A vector comprising the isolated polynucleotide of claim 1.

5. A recombinant DNA construct comprising the polynucleotide of claim 1 operably linked to at least one regulatory sequence.

6. A method for transforming a cell, comprising transforming a cell with the polynucleotide of claim 1.

7. A cell comprising the recombinant DNA construct of claim 5.

8. A plant comprising the recombinant DNA construct of claim 5.

9. A seed comprising the recombinant DNA construct of claim 5.

10. A method of altering the level of sulfur-containing amino acids in a soybean plant which comprises:
(a) transforming a soybean plant cell with the recombinant DNA construct of claim 5;
(b) regenerating a soybean plant from the transformed plant cell of step (a); and
(c) comparing the sulfur-containing amino acid levels of a transformed soybean plant with the sulfur-containing amino acid levels of an untransformed soybean plant.

11. An isolated polynucleotide sequence comprising:
(a) a nucleotide sequence encoding a polypeptide having cysteine synthase activity, wherein the polypeptide has an amino acid sequence of greater than 87.1% sequence identity, based on the Clustal method of alignment, when compared to SEQ ID NO:31;
(b) the full-length complement of the nucleotide sequence of (a); or
(c) all or part of a coding or non-coding region of the isolated polynucleotide sequence comprising any of the nucleotide sequences of (a) or (b) for use in co-suppression or antisense suppression of endogenous nucleic acid sequences encoding polypeptides having cysteine synthase activity.

12. The polynucleotide of claim 11 wherein the amino acid sequence of the polypeptide has at least 95% sequence identity, based on the Clustal method of alignment.

13. The polynucleotide of claim 11 wherein the nucleotide sequence comprises SEQ ID NO: 30.

14. A vector comprising the isolated polynucleotide of claim 11.

15. A recombinant DNA construct comprising the polynucleotide of claim 11 operably linked to at least one regulatory sequence.

16. A method for transforming a cell, comprising transforming a cell with the polynucleotide of claim 11.

17. A cell comprising the recombinant DNA construct of claim 15.

18. A plant comprising the recombinant DNA construct of claim 15.

19. A seed comprising the recombinant DNA construct of claim 15.

20. A method of altering the level of sulfur-containing amino acids in a soybean plant which comprises:
(a) transforming a soybean plant cell with the recombinant DNA construct of claim 15;
(b) regenerating a soybean plant from the transformed plant cell of step (a); and
(c) comparing the sulfur-containing amino acid levels of a transformed soybean plant with the sulfur-containing amino acid levels of an untransformed soybean plant.

21. A method for producing a soybean plant having an altered level of sulfur-containing amino acids which comprises:
(a) crossing an agronomically elite soybean line with the plant of claim 8 or 18; and
(b) screening the seed of progeny plants obtained from step (a) for an altered level of sulfur-containing amino acids compared to an untransformed soybean plant.

22. A method for producing a soybean protein product having an altered level of sulfur-containing amino acids which comprises:
(a) crossing an agronomically elite soybean line with the plant of claim 8 or 18; and
(b) screening the seed of progeny plants obtained from step (a) for an altered level of sulfur-containing amino acids compared to an untransformed soybean plant; and
(c) processing the seed selected in step (b) to obtain the desired soybean protein product.

23. A soybean protein product made from the seed of claim 9 or 19.

24. Food or feed which incorporates the soybean protein product of claim 23.

25. A method of increasing the level of sulfur-containing amino acids in a soybean plant which comprises:
(a) transforming a soybean plant cell with a recombinant DNA construct comprising an isolated polynucleotide sequence comprising:
(i) a nucleotide sequence encoding a polypeptide having cysteine synthase activity, wherein the polypeptide has an amino acid sequence of at least 90% sequence identity, based on the Clustal method of alignment, when compared to one of SEQ ID NO:74;
(ii) the full-length complement of the nucleotide sequence of (i); or
(iii) all or part of a coding or non-coding region of the isolated polynucleotide sequence comprising any of the nucleotide sequences of (i) or (ii) for use in root specific co-suppression or antisense suppression of endogenous nucleic acid sequences encoding polypeptides having cysteine synthase activity wherein said isolated polynucleotide is operably linked to at least one regulatory sequence, said regulatory sequence being a root specific promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

26. A method of increasing the level of sulfur-containing amino acids in a soybean plant which comprises:
(a) transforming a soybean plant cell with a recombinant DNA construct comprising an isolated polynucleotide sequence comprising:
(i) a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31;
for use in overexpressing endogenous nucleic acid sequences encoding polypeptides having cysteine synthase activity in the transformed plant wherein said isolated polynucleotide is operably linked to at least one regulatory sequence, said regulatory sequence being a constitutive promoter.
(b) regenerating a soybean plant from the transformed plant cell of step (a); and
(c) comparing the sulfur-containing amino acid levels of a transformed soybean plant with the sulfur-containing amino acid levels of an untransformed soybean plant.

27. A method of increasing the level of sulfur-containing amino acids in a soybean plant which comprises:
(a) transforming a soybean plant cell with a recombinant DNA construct comprising an isolated polynucleotide sequence comprising a nucleotide sequence encoding a polypeptide having cysteine synthase activity, wherein the polypeptide has an amino acid sequence of greater than 87.1% sequence identity, based on the Clustal method of alignment, when compared to one of SEQ ID NO:31; for use in seed-specific overexpression of endogenous nucleic acid sequences encoding polypeptides having cysteine synthase activity wherein said isolated polynucleotide is operably linked to at least one regulatory sequence, said regulatory sequence being a seed-specific promoter.
(b) regenerating a soybean plant from the transformed plant cell of step (a); and
(c) comparing the sulfur-containing amino acid levels of seeds obtained from a transformed soybean plant with the sulfur-containing amino acid levels of seeds obtained from an untransformed soybean plant.
